SEQUENCE LISTING (200 sequences)

SEQ ID NO: 1 - protein, 9 aa, Homo sapiens
AVTMEPCWK

SEQ ID NO: 2 - protein, 9 aa, Homo sapiens
VTMEPCWKQ

SEQ ID NO: 3 - protein, 9 aa, Homo sapiens
TMEPCWKQI

SEQ ID NO: 4 - protein, 9 aa, Homo sapiens
MEPCWKQID

SEQ ID NO: 5 - protein, 9 aa, Homo sapiens
EPCWKQIDQ

SEQ ID NO: 6 - protein, 9 aa, Homo sapiens
LKNTYRLML

SEQ ID NO: 7 - protein, 9 aa, Homo sapiens
KNTYRLMLY

SEQ ID NO: 8 - protein, 9 aa, Homo sapiens
NTYRLMLYS

SEQ ID NO: 9 - protein, 9 aa, Homo sapiens
TYRLMLYSL

SEQ ID NO: 10 - protein, 9 aa, Homo sapiens
YRLMLYSLR

SEQ ID NO: 11 - protein, 9 aa, Homo sapiens
QQERFFMLA

SEQ ID NO: 12 - protein, 9 aa, Homo sapiens
QERFFMLAW

SEQ ID NO: 13 - protein, 9 aa, Homo sapiens
ERFFMLAWN

SEQ ID NO: 14 - protein, 9 aa, Homo sapiens
RFFMLAWNL

SEQ ID NO: 15 - protein, 9 aa, Homo sapiens
FFMLAWNLE

SEQ ID NO: 16 - protein, 9 aa, Homo sapiens
SAMPRAQLS

SEQ ID NO: 17 - protein, 9 aa, Homo sapiens
AMPRAQLSS

SEQ ID NO: 18 - protein, 9 aa, Homo sapiens
MPRAQLSSA

SEQ ID NO: 19 - protein, 9 aa, Homo sapiens
PRAQLSSAS

SEQ ID NO: 20 - protein, 9 aa, Homo sapiens
RAQLSSASY

SEQ ID NO: 21 - protein, 9 aa, Homo sapiens
LLQEKELPE

SEQ ID NO: 22 - protein, 9 aa, Homo sapiens
LQEKELPEE

SEQ ID NO: 23 - protein, 9 aa, Homo sapiens
QEKELPEEK

SEQ ID NO: 24 - protein, 9 aa, Homo sapiens
EKELPEEKK

SEQ ID NO: 25 - protein, 9 aa, Homo sapiens
KELPEEKKR

SEQ ID NO: 26 - protein, 9 aa, Homo sapiens
KEVLQALKE

SEQ ID NO: 27 - protein, 9 aa, Homo sapiens
EVLQALKEL

SEQ ID NO: 28 - protein, 9 aa, Homo sapiens
VLQALKELA

SEQ ID NO: 29 - protein, 9 aa, Homo sapiens
LQALKELAV

SEQ ID NO: 30 - protein, 9 aa, Homo sapiens
QALKELAVN

SEQ ID NO: 31 - protein, 9 aa, Homo sapiens
GAVFDKCSE

SEQ ID NO: 32 - protein, 9 aa, Homo sapiens
AVFDKCSEL

SEQ ID NO: 33 - protein, 9 aa, Homo sapiens
VFDKCSELR

SEQ ID NO: 34 - protein, 9 aa, Homo sapiens
FDKCSELRE

SEQ ID NO: 35 - protein, 9 aa, Homo sapiens
DKCSELREP

SEQ ID NO: 36 - protein, 9 aa, Homo sapiens
DRSQRDRKE

SEQ ID NO: 37 - protein, 9 aa, Homo sapiens
RSQRDRKEA

SEQ ID NO: 38 - protein, 9 aa, Homo sapiens
SQRDRKEAL

SEQ ID NO: 39 - protein, 9 aa, Homo sapiens
QRDRKEALH

SEQ ID NO: 40 - protein, 9 aa, Homo sapiens
RDRKEALHQ

SEQ ID NO: 41 - protein, 9 aa, Homo sapiens
XXMTAIIEE

SEQ ID NO: 42 - protein, 9 aa, Homo sapiens
XMTAIIEEI

SEQ ID NO: 43 - protein, 9 aa, Homo sapiens
MTAIIEEIV

SEQ ID NO: 44 - protein, 9 aa, Homo sapiens
TAIIEEIVS

SEQ ID NO: 45 - protein, 9 aa, Homo sapiens
AIIEEIVSR

SEQ ID NO: 46 - protein, 9 aa, Homo sapiens
ARQPWKEPT

SEQ ID NO: 47 - protein, 9 aa, Homo sapiens
RQPWKEPTA

SEQ ID NO: 48 - protein, 9 aa, Homo sapiens
QPWKEPTAF

SEQ ID NO: 49 - protein, 9 aa, Homo sapiens
PWKEPTAFQ

SEQ ID NO: 50 - protein, 9 aa, Homo sapiens
WKEPTAFQC

SEQ ID NO: 51 - protein, 9 aa, Homo sapiens
AVTMEPCRK

SEQ ID NO: 52 - protein, 9 aa, Homo sapiens
VTMEPCRKQ

SEQ ID NO: 53 - protein, 9 aa, Homo sapiens
TMEPCRKQI

SEQ ID NO: 54 - protein, 9 aa, Homo sapiens
MEPCRKQID

SEQ ID NO: 55 - protein, 9 aa, Homo sapiens
EPCRKQIDQ

SEQ ID NO: 56 - protein, 9 aa, Homo sapiens
LKNTYRLKL

SEQ ID NO: 57 - protein, 9 aa, Homo sapiens
KNTYRLKLY

SEQ ID NO: 58 - protein, 9 aa, Homo sapiens
NTYRLKLYS

SEQ ID NO: 59 - protein, 9 aa, Homo sapiens
TYRLKLYSL

SEQ ID NO: 60 - protein, 9 aa, Homo sapiens
YRLKLYSLR

SEQ ID NO: 61 - protein, 9 aa, Homo sapiens
QQERFFMPA

SEQ ID NO: 62 - protein, 9 aa, Homo sapiens
QERFFMPAW

SEQ ID NO: 63 - protein, 9 aa, Homo sapiens
ERFFMPAWN

SEQ ID NO: 64 - protein, 9 aa, Homo sapiens
RFFMPAWNL

SEQ ID NO: 65 - protein, 9 aa, Homo sapiens
FFMPAWNLE

SEQ ID NO: 66 - protein, 9 aa, Homo sapiens
SAMPRAQPS

SEQ ID NO: 67 - protein, 9 aa, Homo sapiens
AMPRAQPSS

SEQ ID NO: 68 - protein, 9 aa, Homo sapiens
MPRAQPSSA

SEQ ID NO: 69 - protein, 9 aa, Homo sapiens
PRAQPSSAS

SEQ ID NO: 70 - protein, 9 aa, Homo sapiens
RAQPSSASY

SEQ ID NO: 71 - protein, 9 aa, Homo sapiens
LLQEKELQE

SEQ ID NO: 72 - protein, 9 aa, Homo sapiens
LQEKELQEE

SEQ ID NO: 73 - protein, 9 aa, Homo sapiens
QEKELQEEK

SEQ ID NO: 74 - protein, 9 aa, Homo sapiens
EKELQEEKK

SEQ ID NO: 75 - protein, 9 aa, Homo sapiens
KELQEEKKR

SEQ ID NO: 76 - protein, 9 aa, Homo sapiens
KEVLQALEE

SEQ ID NO: 77 - protein, 9 aa, Homo sapiens
EVLQALEEL

SEQ ID NO: 78 - protein, 9 aa, Homo sapiens
VLQALEELA

SEQ ID NO: 79 - protein, 9 aa, Homo sapiens
LQALEELAV

SEQ ID NO: 80 - protein, 9 aa, Homo sapiens
QALEELAVN

SEQ ID NO: 81 - protein, 9 aa, Homo sapiens
GAVFDKCPE

SEQ ID NO: 82 - protein, 9 aa, Homo sapiens
AVFDKCPEL

SEQ ID NO: 83 - protein, 9 aa, Homo sapiens
VFDKCPELR

SEQ ID NO: 84 - protein, 9 aa, Homo sapiens
FDKCPELRE

SEQ ID NO: 85 - protein, 9 aa, Homo sapiens
DKCPELREP

SEQ ID NO: 86 - protein, 9 aa, Homo sapiens
DRSQRDREE

SEQ ID NO: 87 - protein, 9 aa, Homo sapiens
RSQRDREEA

SEQ ID NO: 88 - protein, 9 aa, Homo sapiens
SQRDREEAL

SEQ ID NO: 89 - protein, 9 aa, Homo sapiens
QRDREEALH

SEQ ID NO: 90 - protein, 9 aa, Homo sapiens
RDREEALHQ

SEQ ID NO: 91 - protein, 9 aa, Homo sapiens
XXMTAIIKE

SEQ ID NO: 92 - protein, 9 aa, Homo sapiens
XMTAIIKEI

SEQ ID NO: 93 - protein, 9 aa, Homo sapiens
MTAIIKEIV

SEQ ID NO: 94 - protein, 9 aa, Homo sapiens
TAIIKEIVS

SEQ ID NO: 95 - protein, 9 aa, Homo sapiens
AIIKEIVSR

SEQ ID NO: 96 - protein, 9 aa, Homo sapiens
ARQPWKEST

SEQ ID NO: 97 - protein, 9 aa, Homo sapiens
RQPWKESTA

SEQ ID NO: 98 - protein, 9 aa, Homo sapiens
QPWKESTAF

SEQ ID NO: 99 - protein, 9 aa, Homo sapiens
PWKESTAFQ

SEQ ID NO: 100 - protein, 9 aa, Homo sapiens
WKESTAFQC

SEQ ID NO: 101 - protein, 5 aa, Homo sapiens
MEPCW

SEQ ID NO: 102 - protein, 5 aa, Homo sapiens
EPCWK

SEQ ID NO: 103 - protein, 5 aa, Homo sapiens
PCWKQ

SEQ ID NO: 104 - protein, 5 aa, Homo sapiens
CWKQI

SEQ ID NO: 105 - protein, 5 aa, Homo sapiens
WKQID

SEQ ID NO: 106 - protein, 5 aa, Homo sapiens
TYRLM

SEQ ID NO: 107 - protein, 5 aa, Homo sapiens
YRLML

SEQ ID NO: 108 - protein, 5 aa, Homo sapiens
RLMLY

SEQ ID NO: 109 - protein, 5 aa, Homo sapiens
LMLYS

SEQ ID NO: 110 - protein, 5 aa, Homo sapiens
MLYSL

SEQ ID NO: 111 - protein, 5 aa, Homo sapiens
RFFML

SEQ ID NO: 112 - protein, 5 aa, Homo sapiens
FFMLA

SEQ ID NO: 113 - protein, 5 aa, Homo sapiens
FMLAW

SEQ ID NO: 114 - protein, 5 aa, Homo sapiens
MLAWN

SEQ ID NO: 115 - protein, 5 aa, Homo sapiens
LAWNL

SEQ ID NO: 116 - protein, 5 aa, Homo sapiens
PRAQL

SEQ ID NO: 117 - protein, 5 aa, Homo sapiens
RAQLS

SEQ ID NO: 118 - protein, 5 aa, Homo sapiens
AQLSS

SEQ ID NO: 119 - protein, 5 aa, Homo sapiens
QLSSA

SEQ ID NO: 120 - protein, 5 aa, Homo sapiens
LSSAS

SEQ ID NO: 121 - protein, 5 aa, Homo sapiens
EKELP

SEQ ID NO: 122 - protein, 5 aa, Homo sapiens
KELPE

SEQ ID NO: 123 - protein, 5 aa, Homo sapiens
ELPEE

SEQ ID NO: 124 - protein, 5 aa, Homo sapiens
LPEEK

SEQ ID NO: 125 - protein, 5 aa, Homo sapiens
PEEKK

SEQ ID NO: 126 - protein, 5 aa, Homo sapiens
LQALK

SEQ ID NO: 127 - protein, 5 aa, Homo sapiens
QALKE

SEQ ID NO: 128 - protein, 5 aa, Homo sapiens
ALKEL

SEQ ID NO: 129 - protein, 5 aa, Homo sapiens
LKELA

SEQ ID NO: 130 - protein, 5 aa, Homo sapiens
KELAV

SEQ ID NO: 131 - protein, 5 aa, Homo sapiens
FDKCS

SEQ ID NO: 132 - protein, 5 aa, Homo sapiens
DKCSE

SEQ ID NO: 133 - protein, 5 aa, Homo sapiens
KCSEL

SEQ ID NO: 134 - protein, 5 aa, Homo sapiens
CSELR

SEQ ID NO: 135 - protein, 5 aa, Homo sapiens
SELRE

SEQ ID NO: 136 - protein, 5 aa, Homo sapiens
QRDRK

SEQ ID NO: 137 - protein, 5 aa, Homo sapiens
RDRKE

SEQ ID NO: 138 - protein, 5 aa, Homo sapiens
DRKEA

SEQ ID NO: 139 - protein, 5 aa, Homo sapiens
RKEAL

SEQ ID NO: 140 - protein, 5 aa, Homo sapiens
KEALH

SEQ ID NO: 141 - protein, 5 aa, Homo sapiens
TAIIE

SEQ ID NO: 142 - protein, 5 aa, Homo sapiens
AIIEE

SEQ ID NO: 143 - protein, 5 aa, Homo sapiens
IIEEI

SEQ ID NO: 144 - protein, 5 aa, Homo sapiens
IEEIV

SEQ ID NO: 145 - protein, 5 aa, Homo sapiens
EEIVS

SEQ ID NO: 146 - protein, 5 aa, Homo sapiens
PWKEP

SEQ ID NO: 147 - protein, 5 aa, Homo sapiens
WKEPT

SEQ ID NO: 148 - protein, 5 aa, Homo sapiens
KEPTA

SEQ ID NO: 149 - protein, 5 aa, Homo sapiens
EPTAF

SEQ ID NO: 150 - protein, 5 aa, Homo sapiens
PTAFQ

SEQ ID NO: 151 - protein, 5 aa, Homo sapiens
MEPCR

SEQ ID NO: 152 - protein, 5 aa, Homo sapiens
EPCRK

SEQ ID NO: 153 - protein, 5 aa, Homo sapiens
PCRKQ

SEQ ID NO: 154 - protein, 5 aa, Homo sapiens
CRKQI

SEQ ID NO: 155 - protein, 5 aa, Homo sapiens
RKQID

SEQ ID NO: 156 - protein, 5 aa, Homo sapiens
TYRLK

SEQ ID NO: 157 - protein, 5 aa, Homo sapiens
YRLKL

SEQ ID NO: 158 - protein, 5 aa, Homo sapiens
RLKLY

SEQ ID NO: 159 - protein, 5 aa, Homo sapiens
LKLYS

SEQ ID NO: 160 - protein, 5 aa, Homo sapiens
KLYSL

SEQ ID NO: 161 - protein, 5 aa, Homo sapiens
RFFMP

SEQ ID NO: 162 - protein, 5 aa, Homo sapiens
FFMPA

SEQ ID NO: 163 - protein, 5 aa, Homo sapiens
FMPAW

SEQ ID NO: 164 - protein, 5 aa, Homo sapiens
MPAWN

SEQ ID NO: 165 - protein, 5 aa, Homo sapiens
PAWNL

SEQ ID NO: 166 - protein, 5 aa, Homo sapiens
PRAQP

SEQ ID NO: 167 - protein, 5 aa, Homo sapiens
RAQPS

SEQ ID NO: 168 - protein, 5 aa, Homo sapiens
AQPSS

SEQ ID NO: 169 - protein, 5 aa, Homo sapiens
QPSSA

SEQ ID NO: 170 - protein, 5 aa, Homo sapiens
PSSAS

SEQ ID NO: 171 - protein, 5 aa, Homo sapiens
EKELQ

SEQ ID NO: 172 - protein, 5 aa, Homo sapiens
KELQE

SEQ ID NO: 173 - protein, 5 aa, Homo sapiens
ELQEE

SEQ ID NO: 174 - protein, 5 aa, Homo sapiens
LQEEK

SEQ ID NO: 175 - protein, 5 aa, Homo sapiens
QEEKK

SEQ ID NO: 176 - protein, 5 aa, Homo sapiens
LQALE

SEQ ID NO: 177 - protein, 5 aa, Homo sapiens
QALEE

SEQ ID NO: 178 - protein, 5 aa, Homo sapiens
ALEEL

SEQ ID NO: 179 - protein, 5 aa, Homo sapiens
LEELA

SEQ ID NO: 180 - protein, 5 aa, Homo sapiens
EELAV

SEQ ID NO: 181 - protein, 5 aa, Homo sapiens
FDKCP

SEQ ID NO: 182 - protein, 5 aa, Homo sapiens
DKCPE

SEQ ID NO: 183 - protein, 5 aa, Homo sapiens
KCPEL

SEQ ID NO: 184 - protein, 5 aa, Homo sapiens
CPELR

SEQ ID NO: 185 - protein, 5 aa, Homo sapiens
PELRE

SEQ ID NO: 186 - protein, 5 aa, Homo sapiens
QRDRE

SEQ ID NO: 187 - protein, 5 aa, Homo sapiens
RDREE

SEQ ID NO: 188 - protein, 5 aa, Homo sapiens
DREEA

SEQ ID NO: 189 - protein, 5 aa, Homo sapiens
REEAL

SEQ ID NO: 190 - protein, 5 aa, Homo sapiens
EEALH

SEQ ID NO: 191 - protein, 5 aa, Homo sapiens
TAIIK

SEQ ID NO: 192 - protein, 5 aa, Homo sapiens
AIIKE

SEQ ID NO: 193 - protein, 5 aa, Homo sapiens
IIKEI

SEQ ID NO: 194 - protein, 5 aa, Homo sapiens
IKEIV

SEQ ID NO: 195 - protein, 5 aa, Homo sapiens
KEIVS

SEQ ID NO: 196 - protein, 5 aa, Homo sapiens
PWKES

SEQ ID NO: 197 - protein, 5 aa, Homo sapiens
WKEST

SEQ ID NO: 198 - protein, 5 aa, Homo sapiens
KESTA

SEQ ID NO: 199 - protein, 5 aa, Homo sapiens
ESTAF

SEQ ID NO: 200 - protein, 5 aa, Homo sapiens
STAFQ

(19) United States
(12) Patent Application Publication
(10) Pub. No.: US 2025/0022536 A1
(43) Pub. Date: Jan. 16, 2025

(54) IMMUNE REPERTOIRE PATTERNS

(71) Applicant: IOGENETICS, LLC, Sun Prairie, WI (US)

(72) Inventors: Robert D. Bremel, Sun Prairie, WI (US); Jane Homan, Sun Prairie, WI (US)

(21) Appl. No.: 18/781,270

(22) Filed: Jul. 23, 2024

Related U.S. Application Data
(63) Continuation of application No. 17/053,955, filed on Nov. 9, 2020.
(60) Provisional application No. 62/669,547, filed on May 10, 2018.
(60) Provisional application No. 62/754,876, filed on Nov. 2, 2018.

(51) Int. Cl. G16B 20/30; G01N 33/68; G16B 50/00

(52) U.S. Cl. CPC G16B 20/30; G01N 33/6818; G16B 50/00; G01N 2570/00

(57) ABSTRACT

The present invention provides methods and systems for identifying and classifying patterns comprising the T cell exposed motifs and the frequencies of such motifs in collections of proteins that make up the human proteome, immunoglobulinome, T cell receptor repertoire or microbiome, and other proteomes of environmental of microbial origin, or subsets thereof. It further provides graphical representations that facilitate comparisons of T cell exposed motif patterns between samples or between time points. The present invention also provides methods and systems for identifying and classifying patterns in repertoires of cells including receptor bearing cells and cells of tissue samples and detecting patterns of utility in diagnosis and monitoring of health and disease.

### CROSS REFERENCE TO RELATED APPLICATIONS

The present application is a continuation of U.S. patent application Ser. No. 17/053,955, filed Nov. 9, 2020, which is a U.S. 371 Application of International Patent Application No. PCT/US2019/031484, filed May 9, 2019, which claims the benefit of U.S. Prov. Appl. 62/669,547 filed May 10, 2018, and U.S. Prov. Appl. 62/754,876, filed Nov. 2, 2018, each of which is incorporated by reference in its entirety.

### SEQUENCE LISTING

The text of the computer readable sequence listing filed herewith, titled “IOGEN_36429_304_SequenceListing” created Jul. 22, 2024, having a file size 172,017 bytes, is hereby incorporated by reference in its entirety.

### BACKGROUND OF THE INVENTION

This invention addresses characterization and utilization of patterns on both sides of the immune interface: the input or antigenic stimulus side and the output or immune response side. On one hand the adaptive immune system is exposed to a wide variety of antigenic stimuli from both inside and outside the body. On the other, the adaptive immune responds to such stimuli by generating a wide diversity of molecules and cellular repertoires. This invention deals with the characterization of these two sets of patterns and how they may be utilized in generating outputs to assist in diagnosis and monitoring health and disease conditions and in designing immunomodulatory interventions.

On the input side, the antigenic stimuli to which the adaptive immune system is exposed come from both endogenous and exogenous sources. The endogenous stimuli are from antigens in proteins that make up the host or self-proteome, comprising all the proteins in the body, the immunoglobulins which comprise a vast diversity of proteins that are in constant turnover to respond to antigenic stimuli, the T cell receptor proteins, the microbiota which are normal commensals of the body. In some cases, the self-proteins include cells which are in tumors. The exogenous stimuli include environmental antigens and pathogens.

The diversity of cellular responses includes, but is not limited to, B cell and T cell responses. B cells diversify as the result of B cell receptor engagement with antigens leading to stimulation, followed by somatic hypermutation and affinity maturation. This in turn leads to a diversity of B cell receptors and immunoglobulins being produced and entering into the repertoire of endogenous antigenic stimuli. The T cell response is determined not only by the presence or absence of a given motif in an antigen, but also the frequency of its occurrence and the duration of T cell encounter. Each source of antigenic stimulation, whether internal or external, provides a different combination of many motifs and a different combination of commonly occurring or rare motifs. This aggregate, or repertoire, of T cell exposed motifs forms a characteristic pattern derived from the peptides making up the combination of proteins in the stimulating source.

On the output or response side, B and T cell clonotype diversity arise as the consequence of antigenic stimulation and each case initiates a feedback loop such that certain clonotypes of cells expand more or less rapidly than others, or may supplant previously dominant clonotypes. Thus, the clonotypic repertoire of each individual is the product of its overall and temporal antigenic exposure or “experience”.

In this invention we provide methods to describe the characteristics of the repertoire patterns in internal and external immune stimuli of healthy and diseased individuals, and in the responding molecules and cells that constitute the immune response. We further provide methods to generate outputs that distinguish said patterns and show how their characteristic patterns may be useful in diagnosis, design and management of interventions and disease monitoring.

### SUMMARY OF THE INVENTION

### Patterns in Antigens

In some preferred embodiments, the present invention is directed to methods of identifying patterns of T cell exposed motifs in multiple proteins, and the utilization of such patterns of motifs to generate outputs that are of utility in diagnosing and managing various disease conditions and interventions to mitigate diseases. These are T cell exposed amino acid motifs that engage T cell receptors as peptides from these proteins of interest serve as T cell epitopes.

In particular, the invention addresses patterns of frequency of occurrence of T cell exposed motifs which may be recognized when a number of proteins, which comprise a proteome, are assembled, the T cell exposed motifs extracted, and their frequency analyzed in comparison to reference databases. The proteomes may be the constituent proteins of a human subject, or other non-human subject, or the proteomes of a microorganism or multiple microorganisms, or may comprise a collection of immunoglobulins or T cell receptors. The reference databases may be derived from analysis of T cell exposed motif frequencies in the human proteome, the human immunoglobulinome, or within a compilation of T cell receptor sequences. The reference databases may also comprise the proteomes of microorganisms including, but not limited to, those making up the microbiome of various tissues, such as the gastrointestinal tract, urogenital tract or the skin. In some cases a total proteome is analyzed, in other instances a partial proteome is analyzed. In some embodiments the proteins in the proteome or partial proteome that is subjected to comparative motif frequency analysis number at least 100, 1000, 5,000 or 10,000 proteins. The upper end of the number of proteins in the proteome is bounded by the total number pf proteins, for example, in the organism, but may also be set at 15,000, 20,000, 30,000, or 50,000 proteins in some preferred embodiments. In yet other instances the proteins analyzed comprise the totality of a human proteome, representation of the total immunoglobulinome, or B cell or T cell receptor repertoire of an individual. In some cases the proteins subject to analysis are assembled from sequencing the microbiome of a subject.

In some instances, the subject from which the proteome analyzed is assembled is a neonate, an infant or a pregnant woman or one intending to become pregnant. In yet other instances the subject from which the proteomes subject to analysis is assembled is an individual over the age of 60 years. In particular embodiments the subject from which a sample is drawn, and proteins sequenced to comprise a proteome for analysis, is suffering from, or suspected to be suffering from a disease, including but not limited to an autoimmune disease, cancer, an inflammatory disease, an allergy, infection or a hematologic disease. In specific instances the individual from which the sample for analysis is derived is undergoing or about to undergo chemotherapy, radiation therapy or immunotherapy. In some of these cases samples may be drawn to enable analysis of the T cell exposed motif repertoires in selected proteomes or immunoglobulinomes before and after therapeutic intervention. In some instances, the subject may be receiving an oral immunonutritional intervention. In one embodiment the subject who provides the sample of a proteome or immunoglobulinome, T cell receptor compilation or microbiome proteome may have been subject to radiation, whether by accident, occupational exposure or as the result of therapeutic intervention.

In additional embodiments, a proteome assembly for T cell exposed motif analysis of the proteins therein may be derived from a biopsy. In some instances, said biopsy is from a tumor or from cancerous cells. In yet other instances the biopsy may comprise normal tissue or cells and the proteins analyzed may provide a comparator of the patterns of T cell exposed motifs in the proteins from a diseased tissue biopsy. In particular instances, analysis of the comparative patterns of T cell exposed motifs in cancerous tissue compared to normal tissue permits the identification of sequences containing T cell exposed motifs which have utility in cancer vaccines. In some cases, the T cell exposed motif for incorporation in a cancer vaccine is further selected by considering the MHC binding affinity to the HLA alleles of the cancer-affected subject from whom the biopsy is derived. In yet further embodiments said binding affinity may be modified by changing amino acids flanking the T cell exposed motifs.

Additional embodiments of the present invention address the analysis of patterns of T cell exposed motifs found in microbial proteomes. The microbial proteomes may be assembled from bacteria or viruses or fungi or parasites. In some instances, the microbial proteome is that of a pathogen; in yet other instances it is of a commensal microbiome. In some instances, said microbial proteomes are those which comprise the gastrointestinal microbiome. In yet other instances the microbial proteomes are those comprising the skin microbiome or the urogenital microbiome. In some embodiments, the microbiome proteomes are collected for analysis from an individual who is affected by a disease. In particular instances said disease may be cancer, autoimmunity, an inflammatory disease, infectious disease, allergy, or a mental disease such as a depression, schizophrenia, autism, or another behavioral disease. In particular instances the microbiome for analysis is derived from an obese individual or a subject affected by another metabolic disease. Samples of microbiota for analysis may be collected from individuals subject to antibiotic or antimicrobial therapy or preventive treatment, chemotherapy or radiation or immunotherapy, including but not limited to checkpoint inhibitor analysis. T cell exposed motif analysis may be applied to microbiome samples from subjects who are undergoing specific interventions to modify their microbiota. In embodiments which address the analysis of the proteomes of microbiome organisms, the relative transcription of the proteins analyzed is determined and the frequency distributions of T cell exposed motifs weighted to reflect the relative transcription.

In some embodiments the bacterial proteomes which are analyzed to determine the patterns of constituent T cell motifs are bacteria which are selected as having utility in modifying the microbiomes of subjects to whom they are administered. In some cases, such bacterial species are referred to as probiotics. In some instances, the analysis of T cell exposed motifs and the patterns of such motifs determined by this process is the basis for selecting a particular bacteria as having a potential beneficial effect in modifying or balancing the microbiome.

In some embodiments a subject may be sampled to obtain sequences of their immunoglobulinome, T cell receptor repertoire, or microbiome on multiple occasions and the patterns of T cell motifs therein analyzed to detect any change in frequency patterns of T cell motifs over time which may be indicative of disease progression or regression or of the efficacy of particular therapeutic interventions or microbiome modifications.

An additional embodiment of this invention provides a graphical representation of the frequency patterns of T cell exposed motifs in a proteome of interest. The graphical representation facilitates recognition and understanding of the changes and differences in patterns of T cell exposed motifs. In some embodiments utilizing such a graphical rendition of T cell motif frequencies, the occurrence of from 5000 to 20,000,000, preferably from 10,000 to 5,000,000, more preferably from 100,000 to 5,000,000, and most preferably about 3.2 million different T cell exposed pentameric motifs are arrayed on a matrix in a consistent order to allow comparison of multiple such matrices between two analysis samples or from samples taken at two timepoints from the same subject. In some preferred embodiments, the matrix arrays may represent the T cell exposed motif frequency patterns in an immunoglobulinome, T cell receptor repertoire, self-proteome or microbial proteome or microbiome. In some preferred embodiments, the matrix arrangements of the T cell exposed motifs are made up of T cell exposed motifs from peptides bound in MHC I molecules; in yet other instances the matrices are made up of T cell exposed motifs exposed from peptides bound in MHC II molecules. To enable comparison between matrices, the individual points are arranged in a consistent order. In some instances, the order of T cell exposed motif array is alphabetical, but in preferred instances the T cell exposed motifs of either MHC I or MHC II T cell exposed motifs are arrayed in order of the principal components of their physical properties. The most preferred embodiment is to array the T cell exposed motif pentamers in the matrix by the first principal component of the physical properties of the pentamer. Coloration or shading of the points or pixels comprising the T cell exposed motif array may be used to indicate the frequency of occurrence of each motif.

In further embodiments of the inventions described herein analysis of the patterns of T cell exposed motifs may be applied to groups of proteins or proteomes that are derived from an environmental organisms, including but not limited to plants, insects and other components making up the allergome. In addition, environmental organisms may be a collection of organisms harvested from a unique or extreme environment. Furthermore, analysis of T cell exposed motif patterns may be applied to collections of proteins in viruses, whether pathogens or endogenous components of the human virome. In yet other embodiments analysis of T cell exposed motif patterns may be applied to parasite proteomes of parasites which are infecting a human host or other subject host of interest.

### Patterns in Repertoires of Immune Responding Cells

In other preferred embodiments, the present invention is directed to methods of identifying patterns of occurrence and frequency of cellular clonotypes arising in the immune response and in tissue samples in various disease conditions.

In some embodiments the present invention provides a method for describing the occurrence and frequency of receptor bearing cells. In some embodiments said receptor bearing cells are B cell or T cells and in other instances the receptor bearing cells carry yet other second receptors, including but not limited to other ligands of which multiple isoforms exist, for example including, but not limited to, programmed death proteins or ligands thereof.

In one embodiment the repertoires of such cells are analyzed by sequencing the nucleic acids of the receptors, as either DNA or RNA, translating to amino acid sequences, categorizing the frequency of unique clonotypes of such cells and organizing in logarithmic-based bins or groups and determining the frequency distribution of the cell clonotypes. In a first embodiment, the invention allows for use of such a process to establish a reference database based on the clonotype repertoires of many individuals and then in a further preferred embodiment to use such a reference database as a comparator for the repertoire of an individual subject. In some embodiments the repertoire of cells is collected by taking a blood sample, for instance where said receptor cells are B cells or T cells. In yet other instances the repertoire of cells is collected by taking a biopsy. In some preferred embodiments the subject whose cellular repertoire is analyzed is affected by an autoimmune disease. In other embodiments the subject whose repertoire is analyzed is affected by cancer. Other conditions that may warrant analysis of repertoires include infections, allergies and other immune dysbiosis.

Analysis of cellular repertoires may, in some embodiments, be done as a means of monitoring progress of a subject following an intervention including, but not limited to, immunotherapy, stem cell transplant, checkpoint inhibitor treatment or microbiome manipulation. In a further embodiment repertoire diversity assessment may be analyzed and characterized as part of a routine monitoring of well-being in a clinically healthy individual. In particular embodiments the repertoire diversity is characteristic of the individual's age. In further embodiments cellular repertoires may be quantified and patterns of occurrence and frequency analyzed based on the presence of other proteins, where such proteins occur in multiple forms such as splice variants or isoforms. In a cancer patient cell clonotypic repertoires may be analyzed to determine the nature and extent of mutagenesis by comparing the frequency patterns of cells bearing specific protein mutations. In each case said clonotype diversity is assessed based on the amino acid sequence as well as the nucleotide sequence.

In some embodiments the clonotypic frequency and diversity based on nucleotide sequences is compared to the clonotypic frequency and diversity based on the amino acid or protein sequences. In some particular embodiments it may be noted that multiple nucleotide sequences result in the same amino acid sequence. In preferred embodiments this is applied to assessment of B cell repertoires. The many nucleotide to one protein sequence relationship indicates a plurality of clonal lines have mutated but all respond to the same B-T cell engagement signals based on the interaction of the T cell receptor and the T cell exposed motif derived from peptides from immunoglobulins. Such many to one relationships of nucleotide sequences to protein sequences may be indicative of daughter clonal lines or may represent bystander selection of clones based on their B-T cell interaction and stimulation therefrom. The degree to which a multiplicity of immunoglobulin nucleotide sequences is transcribed to the same protein may be diagnostic of certain leukemias and will assist in determining an immunotherapeutic intervention which targets B cell displayed sequences.

In some embodiments the B cell clonal diversity pattern, based on the protein sequence when arranged by binning of frequency categories, may be indicative of specific conditions. In some particular embodiments the pattern may be indicative of a B cell neoplasia such as a leukemia or an infection of B cells such as Epstein Barr. As with the molecular repertoire patterns, a further embodiment of the invention also provides for graphical representations to assist in interpretation of patterns of cellular clonotype repertoires.

The subject from which the B and T cells forming the repertoires to be characterized are derived may be a human subject. In other embodiments the subject may be a non-human animal drawn from the group comprising companion animals such as, but not limited to, dogs and cats, livestock, including but not limited to cattle, swine, sheep and goats. The non-human subjects may include, among others, mammals, birds, and fish. The human subjects may include special sub populations defined by, as non-limiting examples, age, reproductive status, sex, disease, exposure to disease causing agents, geographic or ethnic origin.

In preferred embodiments, the analysis is facilitated by utilizing a graphical array as described above.

Accordingly, in some particularly preferred embodiments, the present invention provides methods for generating an output for diagnosing and monitoring the health and disease of an individual subject and designing an immunomodulatory intervention comprising: determining a pattern of occurrence and frequency of T cell exposed motifs contained in a repertoire of proteins to which the individual is exposed as an indicator of the diversity of T cell stimulation provided by the repertoire of proteins; and applying one or more unique features from the unique T cell exposed motif distribution of the frequency pattern to analyze or diagnose the health or disease status of the individual subject or to design or monitor an immunomodulatory intervention for that individual subject. In some preferred embodiments, the frequency pattern is determined by: collecting a biological sample containing the repertoire of proteins, sequencing the proteins of the biological sample, assembling a proteome from the repertoire of proteins, extracting the T cell exposed amino acid motifs from the proteome, determining the frequency of occurrence of each T cell exposed motif, comparing the frequency of occurrence of each T cell exposed motif to the frequency distribution of T cell exposed motifs in a reference database of proteins selected from the group consisting of a human immunoglobulinome reference database, a human T cell receptor sequence reference database, a human proteome reference database, a human microbiome reference database, the proteome of one or more microorganisms other than the microbiome reference database, the allergome, an environmental organism reference database, and a tumor associated mutation reference database, and generating a frequency pattern that identifies the unique T cell exposed motif distribution in the repertoire relative to the reference database.

In some preferred embodiments, the step of comparing the frequency of occurrence of each T cell exposed motif further comprises: indexing each TCEM according to its frequency class in a reference data set of proteins, and comparing the numbers of TCEM in each frequency class in the repertoire of proteins to which the individual is exposed relative to the numbers of TCEM in each frequency class in the reference dataset. In some preferred embodiments, the reference dataset is the human immunoglobulinome. In some preferred embodiments, the step of comparing the frequency of occurrence of each T cell exposed motif further comprises indexing each TCEM according to its quantile score in a reference dataset of proteins, and comparing the numbers of TCEM of each quantile score in the repertoire of proteins to which the individual is exposed relative to the reference dataset.

In some preferred embodiments, the unique features of the unique T cell exposed motif distribution is a loss of TCEM diversity. In some preferred embodiments, the unique features of the unique T cell exposed motif distribution is a gain of TCEM diversity. In some preferred embodiments, the unique features of the unique T cell exposed motif distribution is a change in the number of TCEM of high frequency classes. In some preferred embodiments, the unique features of the unique T cell exposed motif distribution is a change in the number of TCEM of low frequency classes. In some preferred embodiments, the unique features of the unique T cell exposed motif distribution is a change in the number of a group of less than 1000 individual TCEM.

In some preferred embodiments, the immunomodulatory intervention is selected from the group consisting of prophylactic or therapeutic vaccination, administration of CAR-T therapy, administration of a biopharmaceutical drug, administration of chemotherapy, administration of a checkpoint inhibitor, ablation of a population of B or T cells or their progenitors, transplant of B or T cells or their progenitors, radiation, and administration of a dietary supplement or probiotic. In some preferred embodiments, the application of the frequency pattern to analyze the health or disease of an individual is conducted prior to an immunomodulatory intervention. In some preferred embodiments, the application of the frequency pattern to analyze the health or disease of an individual is conducted after an immunomodulatory intervention to monitor the impact thereof on the frequency pattern. In some preferred embodiments, the application of the frequency pattern to analyze the health or disease of the individual subject is conducted as a routine monitoring to assess the diversity of the immune repertoire of the individual subject.

In some preferred embodiments, the reference database is selected from the group consisting of human immunoglobulin variable regions, T cell receptors, and the human proteome. In some preferred embodiments, the repertoire comprises at least 100 proteins. In some preferred embodiments, the repertoire comprises at least 2000 proteins. In some preferred embodiments, the repertoire comprises at least 5000 proteins. In some preferred embodiments, the repertoire of proteins is weighted according to the relative transcription of each protein.

In some preferred embodiments, the patterns are monitored on multiple occasions in an individual to detect changes in the patterns. In some preferred embodiments, the repertoire of proteins is selected from the group consisting of the immunoglobulin sequences of an individual subject, the T cell receptor sequences of an individual subject of an individual subject and a subset of any of the sequences or proteomes. In some preferred embodiments, the individual subject is selected from the group consisting of a neonate, an infant, a pregnant woman, a woman intending to become pregnant. In some preferred embodiments, the individual subject is 60 years or age or older. In some preferred embodiments, the individual subject is at risk of or suffering from a disease condition selected from the group consisting of cancer, autoimmunity, inflammatory diseases, allergies, infections, and a hematologic disease. In some preferred embodiments, the individual is an individual selected from the group consisting of patients subject to chemotherapy, radiation therapy and immunotherapy. In some preferred embodiments, the individual is receiving an oral immunonutritional product. In some preferred embodiments, the individual is subjected to environmental radiation exposure derived from accidental, occupational or iatrogenic exposure.

In some preferred embodiments, the repertoire of proteins is comprised of the proteins present in a tissue sample. In some preferred embodiments, the tissue sample is a biopsy. In some preferred embodiments, the tissue sample is from a tumor. In some preferred embodiments, the tissue sample is from normal tissue. In some preferred embodiments, the repertoires of proteins in normal and tumor tissue are compared to determine differences in the frequency distribution patterns of the T cell exposed motifs in each.

In some preferred embodiments, the repertoire of proteins is comprised of the proteins of the microbiome of an individual subject. In some preferred embodiments, the microbiome comprises bacteria, viruses, fungi, or parasites. In some preferred embodiments, the microbiome is the gastrointestinal microbiome, the skin microbiome or the urogenital microbiome. In some preferred embodiments, the microbiome is collected from an individual affected by a disease selected from the group consisting of cancer, autoimmunity, inflammatory diseases, infectious disease and mental disease. In some preferred embodiments, the microbiome is collected from an individual affected by obesity or other metabolic disease. In some preferred embodiments, the microbiome is collected from an individual who is subject to antibiotic or antimicrobial treatment, chemotherapy, radiotherapy or immunotherapy. In some preferred embodiments, the microbiome is collected from an individual who is subject to interventions to modify their microbiome. In some preferred embodiments, the repertoire of proteins is comprised of the proteins of bacteria from the group comprising bacteria intended to modify the human microbiome. In some preferred embodiments, the bacteria are probiotic. In some preferred embodiments, application of analysis of the T cell exposed motifs present in a bacteria of the group identifies the species pattern of T cell exposed motifs as suitable for administration to a subject.

In some preferred embodiments, the immunomodulatory intervention is selected from the group consisting of a vaccine, a biopharmaceutical, an antibody, an immunonutritional product, and a probiotic.

In some preferred embodiments, the repertoire of proteins is comprised of the proteins of a microbial pathogen. In some preferred embodiments, the microbial pathogen is from the group comprising a bacteria, a virus, a fungus, or a parasite.

In some preferred embodiments, analysis of the pattern of occurrence and frequency of the T cell exposed motifs is used to design an immunomodulatory intervention.

In some preferred embodiments, the methods further comprise generating a graphical output depicting the pattern to facilitate ongoing monitoring. In some preferred embodiments, the pattern is depicted as graphical output comprising an array with about 3.2 million points wherein each point represents a different T cell exposed motif pentamer. In some preferred embodiments, the points are arrayed based on the principal components of the physical properties of the amino acids making up each T cell exposed motif. In some preferred embodiments, the points each representing a T cell exposed motif are categorized based on the frequency of occurrence of each T cell exposed motif in a reference database. In some preferred embodiments, the display depicts the pattern of difference in T cell exposed motif frequency between two analyses.

In some preferred embodiments, the analyses are made on samples taken at different time points from a single subject. In some preferred embodiments, the analyses are made on protein repertoires from samples of cells identified by different functional markers. In some preferred embodiments, the analyses are made on samples taken from different bacterial proteome samples. In some preferred embodiments, the bacterial proteome samples are microbiome samples.

In some preferred embodiments, the repertoire of proteins is comprised of the proteins from an environmental ecosystem external to a human subject. In some preferred embodiments, the environmental ecosystem comprises allergen proteins.

In some preferred embodiments, the present invention provides a cancer vaccine comprising one or more T cell exposed motifs that differentiate the tumor tissue from the normal tissue, as determined as described above. In some preferred embodiments, the cancer vaccine is synthesized and administered to the subject. In some preferred embodiments, the peptide that comprises the one or more T cell motifs that differentiate tumor tissue from normal tissue is further selected to have high affinity MHC binding for the individual from which the tissue sample was derived. In some preferred embodiments, the peptide that comprises the one or more T cell motifs that differentiate tumor tissue from normal tissue is further selected to comprise T cell exposed motifs that occur less frequently than 1 in 2 million T cell exposed motifs in the immunoglobulinome or that are found in the 5% least common motifs in the human proteome.

In some preferred embodiments, the present invention provides methods for generating an output to identify the unique features of the cellular repertoire of an individual subject to diagnose health and disease states and/or to design an immunomodulatory intervention, comprising: determining the pattern of occurrence and frequency of cell clonotypes within repertoires of receptor-bearing cells carried by an individual; and applying the unique features of the frequency distribution of clonotypes to diagnose or monitor the health or disease status of the individual subject or to determine an immunomodulatory intervention for the individual subject. In some preferred embodiments, the frequency pattern is determined by: collecting a biological sample containing a repertoire of receptor-bearing cells, sequencing the nucleic acids of the receptor in the cells and translating each nucleic acid sequence to an amino acid sequence, determining the clonotypic frequency of the cell distribution based on the number of unique receptor amino acid sequences, determining how many representatives of each unique receptor amino acid sequence are in the repertoire, computing the logarithm of the frequency of the representatives at an appropriate base of the frequency, creating bins of an appropriate logarithmic range for tallying clonotypes within each bin range, placing each logarithmic value of the frequency into the appropriate bin; and comparing the clonotypic frequency distribution of receptors in the repertoire of the individual subject to frequency distributions in a reference database of selected from the group consisting of the human B cell receptors, the human T cell receptors, the human proteome, or a reference dataset established from subjects with the same or similar diagnosis.

In some preferred embodiments, the comparing the clonotypic frequency distribution of receptor bearing cells further comprises determining clonotypic diversity by: enumerating the total number of cells in the repertoire, enumerating the number of representatives of each different clonotype, enumerating the number of unique clonotypes, and determining the diversity of the repertoire of receptor bearing cells carried by the individual, and comparing the clonotypic diversity relative to that in a reference dataset.

In some preferred embodiments, the immunomodulatory intervention is selected from the group consisting of prophylactic or therapeutic vaccination, administration of CAR-T therapy, application of a biopharmaceutical drug, administration of chemotherapy, administration of a checkpoint inhibitor, ablation of a population of B or T cells or their progenitors, transplant of B or T cells or their progenitors, radiation, and administration of a dietary supplement or probiotic. In some preferred embodiments, the analysis of the frequency distribution to diagnose or monitor the health or disease status is conducted prior to an immunomodulatory intervention.

In some preferred embodiments, the analysis of the frequency distribution to diagnose or monitor the health or disease status is conducted after an immunomodulatory intervention to monitor the impact thereof on the frequency pattern. In some preferred embodiments, the analysis of the frequency distribution to diagnose or monitor the health or disease status is conducted as a routine monitoring to assess the diversity of the cellular repertoire of the individual subject.

In some preferred embodiments, the methods further comprise making a graphical representation of the clonotypic frequency distributions to facilitate comparison between the repertoire under investigation and the reference database.

In some preferred embodiments, the nucleic acid is a DNA. In some preferred embodiments, the nucleic acid is an RNA. In some preferred embodiments, the receptor bearing cell is a B cell or a T cell. In some preferred embodiments, the receptor is a B cell receptor. In some preferred embodiments, the receptor is a T cell receptor. In some preferred embodiments, the biological sample is a blood sample. In some preferred embodiments, the biological sample is a biopsy sample. In some preferred embodiments, the individual subject is affected by or is at risk of cancer, autoimmune disease, infection, or has been subject to immunotherapy intervention. In some preferred embodiments, the individual subject is clinically healthy.

In some preferred embodiments, the frequency and occurrence of TCEM within the receptors is determined according to the TCEM methods described above.

In some preferred embodiments, the present invention provides methods for generating an output to identify the unique features of the cellular repertoire of an individual subject to diagnose health and disease states and to design an immunomodulatory intervention, comprising
determining the pattern of occurrence and frequency of clonotypes within repertoires of cells expressing a protein of interest; and applying the unique features of the frequency distribution of clonotypes to diagnose or monitor the health or disease status of a subject or to determine an immunomodulatory intervention. In some preferred embodiments, the frequency pattern is determined by: collecting a biological sample containing a repertoire of the cells
sequencing the nucleic acids of the receptor in the cells and translating each nucleic acid sequence to an amino acid sequence, determining the clonotypic frequency of the cell distribution based on the number of unique amino acid sequences of the protein of interest,
determining how many representatives of each unique amino acid sequences of the protein of interest are in the repertoire, computing the logarithm of the frequency of the representatives at an appropriate base of the frequency, creating bins of an appropriate logarithmic range for tallying clonotypes within each bin range, placing each logarithmic value of the frequency into the appropriate bin, and comparing the clonotypic frequency distribution in the repertoire of the individual subject to the frequency distributions in a reference database of selected from the group consisting of the human proteome and a reference dataset established from subjects with the same or similar diagnosis.

In some preferred embodiments, the comparing the clonotypic frequency distribution of receptor bearing cells further comprises determining clonotypic diversity by: enumerating the total number of cells in the repertoire, enumerating the number of representatives of each different clonotype, enumerating the number of unique clonotypes, and determining the diversity of the repertoire of receptor bearing cells carried by the individual, and comparing the clonotypic diversity relative to that in a reference dataset.

In some preferred embodiments, the immunomodulatory intervention is selected from the group consisting of prophylactic or therapeutic vaccination, administration of CAR-T therapy, administration of a biopharmaceutical drug, administration of chemotherapy, administration of a checkpoint inhibitor, ablation of a population of B or T cells or their progenitors, transplant of B or T cells or their progenitors, an immunotherapy targeting the protein of interest, and radiation.

In some preferred embodiments, the analysis of the frequency distribution to diagnose or monitor the health or disease status is conducted prior to an immunomodulatory intervention. In some preferred embodiments, the analysis of the frequency distribution to diagnose or monitor the health or disease status is conducted after an immunomodulatory intervention to monitor the impact thereof on the frequency pattern. In some preferred embodiments, the analysis of the frequency distribution to diagnose or monitor the health or disease status is conducted as a routine monitoring to assess the diversity of the cellular repertoire of the individual subject.

In some preferred embodiments, the nucleic acid is a DNA. In some preferred embodiments, the nucleic acid is an RNA. In some preferred embodiments, the biological sample is a blood sample. In some preferred embodiments, the biological sample is a biopsy sample. In some preferred embodiments, the protein of interest is a surface marker protein. In some preferred embodiments, the surface marker protein is drawn from the group comprising the cluster of differentiation proteins. In some preferred embodiments, the protein of interest is a protein subject to mutagenesis in cancer. In some preferred embodiments, the protein of interest is an enzyme. In some preferred embodiments, the protein of interest occurs as multiple splice variants.

In some preferred embodiments, the individual subject is affected by or is at risk of cancer, autoimmune disease, infection, or has been subject to immunotherapy intervention. In some preferred embodiments, the individual subject is clinically healthy.

In some preferred embodiments, the frequency and occurrence of TCEM within the within the protein of interest in the repertoire is determined by the TCEM methods described above.

In some preferred embodiments, the present invention provides methods for generating an output for diagnosing and monitoring the health and disease of an individual subject and designing an immunomodulatory intervention comprising: identifying patterns of occurrence and frequency of unique immunoglobulin proteins or subsequences thereof within repertoires of B cells of the individual; and applying the analysis of the amino acid and nucleotide sequences a to diagnose or monitor the health or disease status of the individual subject or to design an immunomodulatory intervention for the individual subject. In some preferred embodiments, the frequency pattern is determined by: collecting a biological sample containing a repertoire of the B cells, sequencing the nucleic acids of the receptor in the cells and translating each nucleic acid sequence to an amino acid sequence, determining the frequency of the cell distribution based on the number of unique amino acid sequences of the immunoglobulin or subsequence thereof, determining how many representatives of each unique amino acid sequences of the protein of interest are in the repertoire, and determining how many different nucleotide sequences encode for each unique amino acid sequences in the repertoire.

In some preferred embodiments, the immunomodulatory intervention is selected from the group consisting of prophylactic or therapeutic vaccination, administration of CAR-T therapy, administration of a biopharmaceutical drug, administration of chemotherapy, administration of a checkpoint inhibitor, ablation of a population of B or T cells or their progenitors, transplant of B or T cells or their progenitors, and radiation. In some preferred embodiments, the analysis of the frequency distribution to diagnose or monitor the health or disease status is conducted prior to an immunomodulatory intervention. In some preferred embodiments, the analysis of the frequency distribution to diagnose or monitor the health or disease status is conducted after an immunomodulatory intervention to monitor the impact thereof on the frequency pattern. In some preferred embodiments, the analysis of the frequency distribution to diagnose or monitor the health or disease status is conducted as a routine monitoring to assess the diversity of the B cell repertoire of the individual subject. In some preferred embodiments, the most frequent amino acid sequence is also determined. In some preferred embodiments, the number of unique nucleotide sequences which encode each unique amino acid sequence is determined and a heterogeneity index is assigned to each amino acid sequence. In some preferred embodiments, an immunotherapy intervention is targeted to a multiplicity of clones of B cells which share identical amino acid sequences of their CDR3 or entire variable region. In some preferred embodiments, the shared identical amino acid sequence is in the immunoglobulin heavy chain. In some preferred embodiments, the shared identical amino acid sequence is in the immunoglobulin light chain.

### DESCRIPTION OF THE FIGURES

FIG. 1: TCEM IIA motif patterns in the B cell repertoires of 3 normal healthy donors. Pixel patches show the distribution of 3.2 million TCEM arrayed by first principal component, where the color heat map indicates the number of each motif in the array. The top tier of pixel patches shows the naïve T cells and lower tier the memory T cells as differentiated by cell surface markers

FIG. 2: Shows the differential between the naïve and memory repertoires. The graphic shows the result of the arithmetic difference computed for each of the 2000×1600 TCEM elements in the matrix and then contours applied in a similar manner to FIG. 1.

FIG. 3: Shows a comparison of the frequency of motifs in naive and memory compartment clonotype repertoires of immunoglobulin variable regions of naïve and. Each point represents a single TCEM IIA extracted from the B cell repertoire. Paired comparisons and correlations between M and N compartments showed a characteristic pattern for all three donors. At the peaks these represent about 2⁵ amplification in the Memory pool. This indicates that there is a subset of sequences in the memory pool that undergo substantial amplification.

FIG. 4: Compared the array of TCEM derived from the B cell clonotyes in three normal controls compared to those of six chronic lymphocytic leukemia patients.

FIG. 5: TCEM in B cell repertoires in Chronic lymphocytic leukemia (CLL). Shows unique T cell recognition motif patterns for each patient. Each dot represents a single clonotype. The X axis is the frequency of common motifs in that clonotype and the Y axis is the weighted average of that particular motif in the clonotype.

FIG. 6: The differential motif affinity in a protein pair comprising the native (wild type) protein as compared to the same protein with a non-synonymous mutation giving rise to changes in binding affinity in the region of the mutation.

FIG. 7: Shows the pattern seen when a frame shift occurs giving rise to segment of considerable length where the motifs are different from the wild type sequence until a new stop codon is encountered.

FIG. 8: Shows an example of a protein region wherein a stretch of adjacent overlapping peptides are predicted to have high binding activity in various binding registers for a large number of human MHC alleles with the average over many alleles exceeding 1 std deviation below the mean for all the alleles under consideration.

FIG. 9: Distribution of extremely rare motifs in bacteria dominant in check point inhibitor responder and non-responder patients. Each dot represents a bacterial protein positioned according to its content of FC24 TCEM IIA motifs. A FC24 is a category of motif found less than 1 in 2²³ or less than 1 in 8.388 million B cell clonotypes in a reference database of immunoglobulin variable regions

FIG. 10: Distribution of common motifs in bacteria dominant in check point inhibitor responder and non-responder patients. Each dot represents a bacterial protein positioned according to its content of FC<10 TCEM IIA motifs

FIG. 11: Differences in TCEM IIA distribution in microbiome organisms dominant in anti-PD-1 responders vs non responders. Panel A shows the composite of all identified bacteria in responders and non-responders. Panel B shows results for two species dominant in responder (Bifidobacterium longum) vs non responder (Roseburia intestinalis).

FIG. 12: Comparison of TCEM Frequency categories in probiotics compared to species in non-responding cancer patients, compared to the difference of TCEM frequency categories in responders vs non responders, as shown in Table 1.

FIG. 13: Compares the shared TCEM IIA motifs found in microbiome species found in checkpoint inhibitor responders and non-responders as shown in Table 1, the TCEM IIA in probiotic bacterial species and in the lower tier differentiates which motifs are unique to each group. Probiotic species are listed in Table 2

FIG. 14: Shows arrays of the TCEM 1 diversity patterns from the top 5 hTRAV families of T cells in an individual. 6000-12000 clonotypes are included for each family.

FIG. 15: Frequency distribution of TCEM I in hTRAV subgroup 10

FIG. 16: Using logarithmic binning to elucidate B and T cell repertoire shape

FIG. 17: Hierarchical clustering based on the T cell clonal frequency binning pattern to visualize the cellular frequencies within an individual and to compare and contrast different individuals. A dataset comprising the repertoires of 664 subjects segregated into 30 different subsets based on the repertoire composition.

FIG. 18: Sigmoid curves depicting the T cell repertoires of 664 subjects

FIG. 19: Cumulative distribution pattern of T cell beta variable region clonotypes for 664 subjects that are colored by their CMV serological status

FIG. 20: Comparison of diversity indices related to CMV serostatus

FIG. 21: Cumulative distribution pattern of TCBV clonotypes of 3 subjects with total clonotypes standardized to 100%. All subjects in the A*02 MHC group. Highlighted area shows that 50% of the entire repertoire is in the highly expanded subset of clonotypes. As there is a fixed total pool size there is a substantial loss of diversity as a result. The Shannon entropy and Simpson diversity index that are different measures of repertoire diversity are shown.

FIG. 22: As for FIG. 21 but showing the actual cumulative number of clones (non-standardized)

FIG. 23: Plot of the cumulative distribution (Y axis) of CD4 T cells in the log 2 frequency bins (X axis). These results are for 4 subjects at 6 month (top panel) and 12 month (bottom panel) time points.

FIG. 24: Logistic regression analysis of IgG B cell repertoire on 4 individuals at 12 months. A 3 parameter logistic equation was used to fit the data. The patterns show that subject RA has a dramatically skewed repertoire with a relatively small number of clonotypes but with large number of each. This inflection point for subject RA 2^{8.1} is about greater than 2⁵=32 times greater than subjects RE and RF. This implies that RA has many more cells in several of the high frequency bins.

FIG. 25A-B: Shows suppressive indices in influenza. A. Compared for HA and NA of 3 Influenza A types, based on random sample of 77 H1N1, 14 H2N2, 75 H3N2. Each plot has one type highlighted against background of other types. B. Suppressive indices of all proteins in a set of 61 H1N1 including A/Brevig Mission/1/1918. Arrow shows HA of Brevig Mission is an outlier for predicted

FIG. 26: Compares the frequency distribution of T cell exposed motifs IIA in the immunoglobulinome of a group of 16 hematologic cancer patients with that in in the normal human proteome and gastrointestinal microbiome A) for the aggregate patient group and B) for patient 1 relative to the group and C) for patient 10 relative to the group. The frequency distributions in the reference proteomes of the human and the GI microbiome organisms have been normalized to zero mean unit variance log normal distributions indicated by the dashed lines and are binned by half-standard deviation unit bins. The left-most bin in each histogram represents motifs that are absent from that distribution. Several features can be noted: 1) the human proteome and GI microbiome have different distribution properties, 2) the distribution of TCEM IIa generated by immunoglobulin somatic mutation is skewed toward slightly more rare motifs in both of the reference proteomes, and 3) the immunoglobulin somatic mutations generates broad matches to both reference distributions. At 12 months post transplant patient 1 has generated more matching motifs than patient 10.

FIG. 27: Compares the frequency distribution of T cell exposed motifs IIA in the immunoglobulinome of a group of 16 hematologic cancer patients. The Figure shows the pattern of TCEM IIa distribution before diseased repertoire ablation (time 0) and at 3, 6, and 12 months after bone marrow transplant of HLA matched donors. Frequency of TCEM IIa in the different subjects was standardized by multiplying the frequency of each by 10⁶ and placed in log 2 frequency bins (x-axis). The y-axis is the relative proportion of the total distribution found in any of the individual bins. The distributions are modeled as a 4-normal distribution mixture (red line). The dashed lines at generated from the 12 month data model and are centered on the underlying modeled distribution means. These points are used as reference frequencies in the other distributions and show the expansion of more rare motifs over time.

FIG. 28: TRBV Repertoire Shapes Healthy Subjects by Age

FIG. 29: Comparison of B cell amino acid repertoire diversity in normal and leukemic patients based on log₂ binning of cells per million.

FIG. 30: Shows hierarchical clustering of CDR3 sequences of immunoglobulin heavy and light chains for two patients with diffuse large B-cell lymphoma. FIG. 30 provides data pertaining to light chains. The figure shows a hierarchical clustering based first on nucleotide sequence (A), then on CDR amino acid sequence (B) and thirdly on whole variable region (C). In the left hand panel of each the unique nucleotide sequences are randomly colored to indicate the diversity (A). In the right hand panel the unique nucleotide sequences are colored to indicate the frequency of each unique. sequence (A′). Multiple nucleotide sequences correspond to each CDR amino acid sequence and each unique CDR sequence is found in a few total variable regions. Hence many unique A>each unique B>few unique C. Patterns for light and heavy chains are similar but unrelated.

FIG. 31: Shows hierarchical clustering of CDR3 sequences of immunoglobulin heavy and light chains for two patients with diffuse large B-cell lymphoma. FIG. 31 provides data pertaining to light chains. The figure shows a hierarchical clustering based first on nucleotide sequence (A), then on CDR amino acid sequence (B) and thirdly on whole variable region (C). In the left hand panel of each the unique nucleotide sequences are randomly colored to indicate the diversity (A). In the right hand panel the unique nucleotide sequences are colored to indicate the frequency of each unique. sequence (A′). Multiple nucleotide sequences correspond to each CDR amino acid sequence and each unique CDR sequence is found in a few total variable regions. Hence many unique A>each unique B>few unique C. Patterns for light and heavy chains are similar but unrelated.

FIG. 32: Shows hierarchical clustering of CDR3 sequences of immunoglobulin heavy and light chains for two patients with diffuse large B-cell lymphoma. FIG. 32 provides data pertaining to light chains. The figure shows a hierarchical clustering based first on nucleotide sequence (A), then on CDR amino acid sequence (B) and thirdly on whole variable region (C). In the left hand panel of each the unique nucleotide sequences are randomly colored to indicate the diversity (A). In the right hand panel the unique nucleotide sequences are colored to indicate the frequency of each unique. sequence (A′). Multiple nucleotide sequences correspond to each CDR amino acid sequence and each unique CDR sequence is found in a few total variable regions. Hence many unique A>each unique B>few unique C. Patterns for light and heavy chains are similar but unrelated.

FIG. 33: Shows hierarchical clustering of CDR3 sequences of immunoglobulin heavy and light chains for two patients with diffuse large B-cell lymphoma. FIG. 33 provides data pertaining to light chains. The figure shows a hierarchical clustering based first on nucleotide sequence (A), then on CDR amino acid sequence (B) and thirdly on whole variable region (C). In the left hand panel of each the unique nucleotide sequences are randomly colored to indicate the diversity (A). In the right hand panel the unique nucleotide sequences are colored to indicate the frequency of each unique. sequence (A′). Multiple nucleotide sequences correspond to each CDR amino acid sequence and each unique CDR sequence is found in a few total variable regions. Hence many unique A>each unique B>few unique C. Patterns for light and heavy chains are similar but unrelated.

FIG. 34: Occurrence of multiple nucleotide coding found in 39.73 million immunoglobulin sequences from normal patients. Right hand column shows how many nucleotide sequences encode, Count column shows instances of this number of alternate nucleotide codes.

FIG. 35: Shows frequency distribution of TCEM (TCEM 1, IIA, IIB) for 848 commonly recognized allergens of animal, plant, fungal, insect, mite helminth and contact sources compared to the frequency of the same TCEM in the human proteome. The mean for the human proteome is zero, showing that the allergens comprise significantly more TCEM that are rare in the human proteome.

FIG. 36: Shows the frequency classes of TCEM IIA for several individual allergen proteins from peanuts (top) and cats (bottom). TCEM class 24 are those which occur less commonly than 1 in 8,388,608 (2²⁴) in the human immunoglobulinome.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (www.ebi.ac.uk/interpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 20 or less amino acids and “polypeptide” is used to refer to a sequence of greater than 20 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I,) and the maturation of precursor proteins (e.g. enteropeptidase, furin). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

“B cell core peptides” or “core pentamer” when used herein refers to the central 5 amino acid peptide in a predicted B cell epitope sequence. Said B cell epitope may be evaluated by predicting the binding of across a series of 9-mer windows, the core pentamer then is the central pentamer of the 9-mer window

As used herein, the term “predicted B-cell epitope” refers to a polypeptide sequence that is predicted to bind to a B-cell receptor by a computer program, for example, as described in PCT US2011/029192, PCT US2012/055038, and US2014/014523, each of which is incorporated herein by reference, and in addition by Bepipred (Larsen, et al., Immunome Research 2:2, 2006.) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally.

As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, a “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allotype, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order.

As used herein, the term “allergen” refers to an antigenic substance capable of producing immediate hypersensitivity and includes both synthetic as well as natural immunostimulant peptides and proteins. Allergen includes but is not limited to any protein or peptide catalogued in the Structural Database of Allergenic Proteins database http://fermi.utmb.edu/SDAP/index.html

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “consensus protease cleavage site” refers to an amino acid sequence that is recognized by a protease such as trypsin or pepsin.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “Ka”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷M⁻¹ to 2×10⁶M⁻¹.

As used herein, the terms “weak binder” and “weak binding” and “low affinity” refer to a binding pair or describe a binding pair that have an affinity of less than 2×10⁶M⁻¹ (equivalent to a dissociation constant of 500 nM Kd)

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, NY). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In other embodiments, suitable monoclonal antibodies, including recombinant chimeric monoclonal antibodies and chimeric monoclonal antibody fusion proteins are prepared as described herein.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom, M., and Eriksson, L., Chemometrics and Intelligent Laboratory Systems 2001. 58: 109-130; Multivariate and Megavariate Data Analysis Basic Principles and Applications (Parts I&II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference, unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors.

As used herein the term “biofilm” refers to an aggregation of microorganisms (e.g., bacteria) surrounded by an extracellular matrix or slime adherent on a surface in vivo or ex vivo, wherein the microorganisms adopt altered metabolic states.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.).

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

The terms “bacteria” and “bacterium” refer to prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red. In some embodiments, the bacteria are those capable of causing disease (pathogens) and those that cause product degradation or spoilage.

“Strain” as used herein in reference to a microorganism describes an isolate of a microorganism (e.g., bacteria, virus, fungus, parasite) considered to be of the same species but with a unique genome and, if nucleotide changes are non-synonymous, a unique proteome differing from other strains of the same organism. Typically strains may be the result of isolation from a different host or at a different location and time but multiple strains of the same organism may be isolated from the same host.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1, 2, 3, 9). In the case of MHC-II molecules two formats of GEM are most common comprising amino acids (−3, 2, −1, 1, 4, 6, 9, +1, +2, +3) and (−3, 2, 1, 2, 4, 6, 9, +1, +2, +3) based on a 15-mer peptide with a central core of 9 amino acids numbered 1-9 and positions outside the core numbered as negative (N terminal) or positive (C terminal).

“Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced by www.imgt.org [1]. “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

“Germline motif” as used herein describes the amino acid subsets that are found in germline immunoglobulins. Germline motifs comprise both GEM and TCEM motifs found in the variable regions of immunoglobulins which have not yet undergone somatic hypermutation.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Illustrative examples of immunopathologies include, but are not limited to, B-cell lymphoma, T-cell lymphomas, Systemic Lupus Erythematosus (SLE), allergies, hypersensitivities, immunodeficiency syndromes, radiation exposure or chronic fatigue syndrome.

“Obverse” as used herein describes the outward directed face or the side facing outwards. Hence, in the context of a pMHC complex, the obverse side is that face presented to the T-cell receptor and comprises the space-shape made up of the TCEM and the contiguous and surrounding outward facing components of the MHC molecule that will be different for each different MHC allele.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“Somatic hypermutation” (SHM), as used herein refers to the process by which variability in the immunoglobulin variable region is generated during the proliferation of individual B-cells responding to an immune stimulus. SHM occurs in the complementarity determining regions.

“T-cell exposed motif” (TCEM), as used herein, refers to the sub set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in said MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“Tregitope” as used herein describes an epitope to which a Treg or regulatory T-cell binds.

“uTOPE™ analysis” as used herein refers to the computer assisted processes for predicting binding of peptides to MHC and predicting cathepsin cleavage, described in PCT US2011/029192, PCT US2012/055038, and US2014/01452, each of which is incorporated herein by reference.

“Framework region” as used herein refers to the amino acid sequences within an immunoglobulin variable region which do not undergo somatic hypermutation.

“Isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform.

“Class switch recombination” (CSR) as used herein refers to the change from one isotype of immunoglobulin to another in an activated B cell, wherein the constant region associated with a specific variable region is changed, typically from IgM to IgG or other isotypes.

“Immunostimulation” as used herein refers to the signaling that leads to activation of an immune response, whether said immune response is characterized by a recruitment of cells or the release of cytokines which lead to suppression of the immune response. Thus immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

“IGHV” as used herein is an abbreviation for immunoglobulin heavy chain variable regions.

“IGLV” as used herein is an abbreviation for immunoglobulin light chain variable regions “Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Anchor peptide”, as used herein, refers to peptides or polypeptides which allow binding to a substrate to facilitate purification or which facilitate attachment to a solid medium such as a bead or plastic dish or are capable of insertion into a membrane of a cell or liposome or virus like particle. Among the examples of anchor peptides are the following, which are considered non limiting, his tags, immunoglobulins, Fc region of immunoglobulin, G coupled protein, receptor ligand, biotin, and FLAG tags

“Cytotoxin” or “cytocide” as used herein refers to a peptide or polypeptide which is toxic to cells and which causes cell death. Among the non-limiting examples of such polypeptides are RNAses, phospholipase, membrane active peptides such as cercropin, and diphtheria toxin. Cytotoxin also includes radionuclides which are cytotoxic.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

As used herein “oncoprotein” means a protein encoded by an oncogene which can cause the transformation of a cell into a tumor cell if introduced into it. Examples of oncoproteins include but are not limited to the early proteins of papillomaviruses, polyomaviruses, adenoviruses and herpesviruses, however oncoproteins are not necessarily of viral origin.

“Label peptide” as used herein refers to a peptide or polypeptide which provides, either directly or by a ligated residue, a colorimetric, fluorescent, radiation emitting, light emitting, metallic or radiopaque signal which can be used to identify the location of said peptide. Among the non-limiting examples of such label peptides are streptavidin, fluorescein, luciferase, gold, ferritin, tritium,

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used here in “virome” comprises the viruses present in a human subject, latently chronically or during acute infection, or a sub set thereof made up of viruses of a particular taxonomic group or of the viruses located in a particular tissue or organ.

“Immunoglobulinome” as used herein refers to the total complement of immunoglobulins produced and carried by any one subject.

The terms “surfome”, “sheddome”, and “secretome” as used herein refer to subsets of a proteome which are respectively exposed on a cell surface, shed from the surface of a cell or organism into the surrounding milieu or actively secreted by an organism or cell into the surrounding milieu.

As used herein “allergome” refers to all proteins which may give rise to allergies. This includes proteins recorded in allergen datasets such as that represented at www.allergome.com, http://www.allergenonline.org/, http://comparedatabase.org/www.allergen.org as well as included in Uniprot, Swiss prot, etc.

As used herein “pixel patch” is an ordered array of 3.2 million unique pentamer TCEMs which allows comparison of frequency patterns of TCEM within a protein or a repertoire of proteins. The array may be ordered alphabetically or according to the first principal component or according to any other unique identifying metric that will allow the count of all TCEM, whether TCEM I TCEM IIA or IIB, to be compared. One convenient modulo 20 matrix arrangement to allow for an arrangement of 2000×1600×20 amino acids.

As used herein the term “repertoire’ is used to describe a collection of molecules or cells making up a functional unit or whole. Thus, as one non limiting example, the entirely of the B cells or T cells in a subject comprise its repertoire of B cells or T cells. The entirety of all immunoglobulins expressed by said B cells are its immunoglobulinome or the repertoire of immunoglobulins. A collection of proteins or cell clonotypes which make up a tissue sample, an individual subject or a microorganism may be referred to as a repertoire.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“TRAV” as used herein refers to the T cell receptor alpha variable region family or allele subgroups and “TRBV” refers to T cell receptor beta variable region family or allele subgroups as described in IMGT http://imgt.org/IMGTrepertoire/Proteins/index.php#C http://imgt.org/IMGTrepertoire/Proteins/taballeles/human/TRA/TRAV/Hu TRAVall. html TRAV comprises at least 41 subgroups, with some having sub-subgroups. TRBV comprises at least 30 subgroups. Most combinations of alpha and beta variable region subgroups are encountered. “hTRAV” refers to human TRAV.

As used here in a “receptor bearing cell” is any cell which carries a ligand binding recognition motif on its surface. In some particular instances a receptor bearing cell is a B cell and its surface receptor comprises an immunoglobulin variable region, said immunoglobulin variable region comprising both heavy and light chains which make up said receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, and a nutritional or dietary supplement. The intervention may also include radiation or chemotherapy to ablate a target group of cells. The impact on the immune response may be to stimulate or to down regulate.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed at https://www.uniprot.org/docs/cdlist

As used herein “microbiome” refers to the constellation of commensal microorganisms found within the human or other host body, inhabiting sites such as the gastrointestine, skin the urogenital tract, the oral cavity, the upper respiratory tract. While most frequently referring to bacteria, the microbiome also may include the viruses in these sites, referred to as the “virome”, or commensal fungi.

As used herein “tumor associated mutations” refers to all nucleotide or amino acid mutations detected in a tumor. In some cases the tumor associated mutations are commonly found within many patients with a particular tumor type. In other cases tumor associated mutations may be unique to a specific patient. In other instances different patients may carry different tumor associated mutations r in the same protein.

“Repertoire” as used herein refers to the entirety of data points in a collection which maybe, but is not limited to a tissue sample, a proteome, an immunoglobulin a microorganism and wherein said data points may include, but are not limited to, sequences of amino acids or nucleotides, amino acid motifs, nucleotide motifs, cells, or microorganisms

“Pattern” as used herein means a characteristic or consistent distribution of data points.

As used herein a “frequency pattern” is a data set that displays the frequency of TCEMs in a repertoire of proteins from a proteome associated with an individual subject as compared to the frequency of those TCEMs in a reference database. Particular TCEMs, or groups of TCEMs, within the subject's repertoire may occur at the same, lower or higher frequencies than the corresponding TCEMs in the reference database. The frequency pattern allows identification and categorization of unique TCEMs and/or patterns of TCEMs (i.e., unique features of unique TCEM features). The term “frequency pattern” as used herein is also used to describe the distribution of cellular clonotypes within a repertoire of cells from an individual subject, as compared to the frequency of the cellular clonotypes in a reference database. Particular clonotypes, or groups of clonotypes, within the subject's repertoire may occur at the same, lower or higher frequencies than the corresponding cellular clonotypes in the reference database. The frequency pattern allows identification and categorization of unique patterns of clonotypes. In some embodiments, a “frequency class” or “frequency classification” is assigned to a TCEM motif or to a cellular clonotype based on its frequency as described elsewhere herein.

As used herein “clonotype” is a line of cells derived from a committed or fully differentiated progenitor. In the case of T cells and somatic cells other than B cells, a clonotype of cells has a common genotype, i.e. comprises a common nucleotide sequence. Clonotypes with different nucleotide sequences may express a protein of identical amino acid sequence as a result of different codon utilization. Hence multiple genotypes may lead to a shared phenotype among such clonotypes. In B cells, somatic mutation results in a differentiated cell line comprising a nucleotide sequence that expresses antibodies of one isotype and variable region sequence; this is a B cell clonotype.

As used herein “clonotypic diversity” refers to the distribution of the total number of cells in a repertoire among all unique clonotypes in a repertoire. Hence, if a repertoire has 1 million cells, but these comprise 400,000 of clonotype 1 and 600,000 of clonotype 2, the repertoire has a low clonotypic diversity. If the 1 million cells are distributed as 10 each of 100,000 unique clonotypes the repertoire has a high clonotypic diversity.

As used herein “many to one” describes a relationship in which one protein or peptide sequence is encoded be many different synonymous nucleotide sequences.

As used herein “IVIG” refers to intravenous immunoglobulin used as a therapeutic intervention.

### DESCRIPTION OF THE INVENTION

This invention addresses characterization and utilization of patterns on both sides of the immune interface: the input or antigenic stimulus side and the output or immune response side. On one hand the adaptive immune system is exposed to a wide variety of antigenic stimuli from both inside and outside the body. On the other, the adaptive immune responds to such stimuli by generating a wide diversity of molecules and cellular repertoires. This invention deals with the characterization of these two sets of patterns and how they may be utilized in generating outputs to assist in diagnosis and monitoring health and disease conditions and in designing immunomodulatory interventions.

On the input side, the antigenic stimuli to which the adaptive immune system is exposed come from both endogenous and exogenous sources. The endogenous stimuli are from antigens in proteins that make up the host or self-proteome, comprising all the proteins in the body, the immunoglobulins which comprise a vast diversity of proteins that are in constant turnover to respond to antigenic stimuli, the T cell receptor proteins, the microbiota which are normal commensals of the body. In some cases, the self proteins include cells which are in tumors. The exogenous stimuli include environmental antigens and pathogens.

The diversity of cellular responses includes, but is not limited to, B cell and T cell responses. B cells diversify as the result of B cell receptor engagement with antigens leading to stimulation, followed by somatic hypermutation and affinity maturation. This in turn leads to a diversity of B cell receptors and immunoglobulins being produced and entering into the repertoire of endogenous antigenic stimuli. The T cell response is determined not only by the presence or absence of a given motif in an antigen, but also the frequency of its occurrence and the duration of T cell encounter. Each source of antigenic stimulation, whether internal or external, provides a different combination of many motifs and a different combination of commonly occurring or rare motifs. This aggregate, or repertoire, of T cell exposed motifs forms a characteristic pattern derived from the peptides making up the combination of proteins in the stimulating source.

The discrimination between self and non-self is largely dependent on the T-cell responses and is the combination of peptide binding by the host's genetically determined MHC molecules and the recognition by T cells of the amino acid motifs comprised in peptides which are bound by MHC molecules and exposed to T-cells in the context of the MHC molecules. Which peptides become available for MHC binding is determined by endopeptidase action in the antigen presenting cells, including but not limited to cathepsin cleavage.

A peptide bound into a MHC molecule, whether MHC I or MHC II, typically only exposes a motif of five amino acids to the T cell receptor (TCR). The TCR then recognizes that pentamer as a unique signal within the context of the histotope, or outward facing surface of the MHC. There are three different arrangements of such pentameric motifs. However, given the limitation of twenty amino acids arranged in a pentameric motif, each arrangement is restricted to 20⁵ or 3.2 million possibilities. Given this relatively small number there is inevitably a high degree of sharing of motifs among all the internal and external sources of antigenic stimulation. The T cell response is determined not only by the presence or absence of a given motif, but also the frequency of its occurrence and the duration of T cell encounter, where the latter is determined by the dwell time in the MHC groove. This in turn is affected by the MHC allele of the individual, where different HLA alleles will lead to longer or shorter dwell times based on binding affinity. Each source of antigenic stimulation to which an individual host is exposed provides a different combination of many motifs and hence a different combination of commonly occurring or rare motifs. This aggregate mosaic pattern or repertoire of T cell exposed motifs forms a characteristic pattern derived from the combination of proteins in the stimulating source. Hence, one bacteria, made up of, for example, 3000 proteins in aggregate comprising over a million different T cell exposed motifs, will present a different characteristic pattern from the patterns arising from another species or genus of bacteria with a similar number of proteins and T cell exposed motifs. These patterns may vary even among isolates of the same species of bacteria. The collective diverse immunoglobulins (immunoglobulinome) of one individual will comprise a different overall composition of T cell exposed motifs from their neighbor who has a different immune exposure history, or from an individual suffering from cancer. Similarly, the different T cell repertoires of two individuals will generate a different pattern of motifs derived from the T cell receptors.

On the output or response side, B and T cell clonotype diversity arise as the consequence of antigenic stimulation and each case initiates a feedback loop such that certain clonotypes of cells expand more or less rapidly than others, or may supplant previously dominant clonotypes. Thus, the clonotypic repertoire of each individual is the product of its overall and temporal antigenic exposure or “experience”.

Examining the patterns of diversity and frequency of cellular clonotypes, or the use of T cell exposed motifs, and their counterparts binding the MHC grooves will characterize the source and the consequent T cell stimulation pattern of the host immune system. Comparison of patterns over time within an individual subject or between subjects may provide indicators of the T cell repertoire condition and diversity. Such patterns in turn will indicate how robust an immune response may be and whether said response will be one of T cell upregulation or suppression. Determination of, and examination of, the patterns of molecular stimuli and cellular responses can therefore identify characteristics that drive pathogenesis, identify potential modes of intervention, and allow diagnosis and monitoring of patients.

By analogy, in human speech the vocabulary, sentence patterns and cadence used, irrespective of subject matter or particular individual words, provide patterns that will distinguish two speakers and provide information on education, musicality, intelligence, social background, age and health. Comparison of such patterns over time may show pattern changes diagnostic of certain diseases or ageing.

In a prior application, the present inventors addressed the identification, occurrence and distribution of T cell exposed motifs (TCEM) in individual proteins (See, e.g., PCT/US2015/039969, incorporated by reference herein in its entirety) and the applications of analysis thereof in vaccine design and other interventions which focus on individual proteins. The present invention differs from what has been previously described and provides significant improvements by taking a higher level view, to examine how analysis of large repertoires of proteins enables the identification of distinctive repertoire-wide patterns which provide insight and guidance in observing and managing the human T cell repertoire and balance thereof. Continuing the analogy to speech above, whereas our prior specification addressed individual proteins and peptides (comparable to words and how to compile a dictionary) the present invention addresses patterns of peptide repertoire patterns and cellular clonotypes and the interpretation thereof (comparable to patterns of speech, vocabulary, poetry and prose and the information derived therefrom described above).

### T Cell Exposed Motifs (TCEM):

The major histocompatibility molecules, or MHC, bind peptides created by enzymatic processing of proteins by cathepsins or the proteasome. Class I or MHC I, which bind and stimulate CD8+ cytotoxic T cells (CTL) bind short peptides of 8-11 amino acids and expose a TCEM of five continuous amino acids. Within a 9 mer these amino acids are in are positions ˜˜˜45678˜, while positions 123˜˜˜˜˜9 are amino acids facing inwards as the MHC groove exposed motifs or pocket positions. Class II or MHC II bound peptides stimulate CD4+ T cells including T helper cells. The peptides which bind MHC II are longer and more variable as the grooves are more open and tolerant of different lengths; typically peptides of 13-20 amino acids and most typically 15 amino acids bind MHC II. The T cell exposed motifs adopt two configurations, with respect to a central core of 9 amino acids they are at positions ˜2,3˜5˜7,8˜ or −1˜3˜5˜7,8˜, again with the interspersed amino acids forming the groove exposed motifs [2, 3]

Of the 3.2 million possible TCEM in each pentameric recognition pattern (20 amino acids in five positions=20⁵) each is present at a different frequency in the immunoglobulinome, T cell receptor, self-proteome (other than immunoglobulins) and gastrointestinal microbiome [4, 5]. Hence reference datasets of T cell exposed motifs and their normal frequency of occurrence can be established for these sources of T cell stimulation (See, e.g., PCT/US2015/039969, incorporated by reference herein in its entirety). Having established reference data sets of normal distributions then enables comparison of any set of T exposed motifs to these reference distributions.

T regulatory T cells or “Treg”s are immunosuppressive T cells elicited in some particular instances by IL10 and which act to suppress, down regulate or modulate the immune response. A necessary condition to elicit a Treg response is a high frequency of pMHC:TCR signaling [6]. Those TCEM which occur at high frequency are likely to elicit a large cognate T cell population and, when the TCEM is also associated with a groove exposed motif that favors binding to the MHC, will create the high frequency of signaling conditions that are conducive to formation of Treg. The occurrence of many common or high frequency motifs within a repertoire of TCEMs can therefore be indicative of a situation that leads to immune suppression or modulation. At the other extreme, the presence in a repertoire of proteins of many TCEM motifs that are rare is indicative of an upregulatory or proinflammatory condition.

The present invention addresses the applications of analyses of T cell exposed motifs to gain insights into the characteristics of multiple protein repertoires. These include:
The human IgV repertoire as an indicator of the breadth of T cell repertoire in various conditions.
The T cell receptor sequence diversity as a direct measure of T cell diversity
The microbiome repertoire and that of the constituent bacteria, thereby enabling selection of particular bacteria, and other microorganisms, and understanding of the roles of microbiome constituents as T repertoire stimuli
The repertoire of TCEM in comparative tissue samples in cancer—to enable selection of neoepitopes
The analysis of other repertoires to which the human immune system is exposed including but not limited to the proteomes of pathogenic bacteria, fungi, endoparasites, virome, and other potentially pathogenic microorganisms, environmental immunogenic proteins including, but not limited to, the allergome.

### Immunoglobulin Repertoires

The immunoglobulinome is a particularly valuable reference dataset of TCEM frequency. B cells not only enzymatically cleave proteins and present peptides derived from a stimulating exogenous antigen to T cells, but also enzymatically cleave their endogenous immunoglobulins yielding peptides which are presented on MHC and elicit T cell help [7, 8]. As the diversity and turnover of the immunoglobulinome far exceeds that of the rest of the self-proteome, and the total volume of immunoglobulin proteins in the body is large, the continual processing, presentation and T cell engagement arising from the immunoglobulinome is apparently a dominant factor in balancing the T cell repertoire [4].

As the host is exposed to more or less diversity of internal and external immune stimuli, the diversity and balance of the immunoglobulin population changes. As a new immunogen is encountered it will cause expansion of the responsive B cell clone at the expense of others. Hence the immunoglobulin repertoire is different in individuals with autoimmune diseases, acute infections or allergies. In one embodiment of the present invention we identify TCEM patterns in the immunoglobulin of a subject. In some embodiments said patterns are in MHC I TCEM, in others in MHC II TCEM. In some embodiments the subject is an apparently healthy individual. In yet others the individual may have been exposed to an infection, by a virus, bacteria, fungus or other microorganism or be infected by a eukaryotic parasite. In some cases the infected individual may have been treated with an antimicrobial drug, antibiotic or anthelmintic and the invention described allows monitoring of the changes in the TCEM patterns in the immunoglobulinome and in the B-cells which generate said immunoglobulinome. In yet other instances the individual in which the pattern of TCEM in the immunoglobulinome is studied is affected by an autoimmune disease, including but not limited to, one of the following: celiac disease, narcolepsy, rheumatoid arthritis and multiple sclerosis, ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune uveitis, Bullous Pemphigoid, Castleman's disease, Celiac disease, Cogan syndrome, Cold agglutinin disease, Crohn's Disease, Dermatomyositis, Diabetes mellitus type 1, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Pemphigus vulgaris, Polymyositis, Primary biliary cirrhosis, Psoriasis, Psoriatic Arthritis, Relapsing polychondritis, Rheumatoid arthritis, Sjögren's syndrome, Temporal arteritis, Ulcerative Colitis, Vasculitis, and Wegener's granulomatosis.

In another embodiment the present invention allows monitoring of the TCEM pattern in the immunoglobulinome as an indicator of the T cell repertoire diversity in individuals who are subject to inflammatory diseases such as but not limited to ulcerative bowel disease, Crohn's disease and rheumatoid arthritis and arthritis of other etiologies.

In yet other embodiments the individual in which we analyze the immunoglobulin TCEM patterns is affected by cancer, including but not limited to cancers affecting the B and T cells but also cancers affecting other tissues. In both instances the invention enables the monitoring of the repertoires of TCEM as an indicator of the diversity and repertoire of the T cells essential to mount an immune response. In the particular case of B cell leukemias, the B cell population is dominated by the clonal population of the tumor, with the usual diversity supplanted by a small number of neoplastic clones secreting a limited number of immunoglobulins. The present invention provides a means of identifying those clones and monitoring their expansion or contraction following medical intervention.

In some particular instances, the individual affected by an autoimmune disease or a cancer is the subject of an immunotherapeutic or immunomodulatory intervention, including but not limited to a vaccine, a biotherapeutic antibody-based therapy such as, but not limited to, trastuzumab, rituximab or other antibody-based therapeutic intervention. In yet other cases the individual is undergoing therapy with a checkpoint inhibitor drug. A further category of individuals in which the invention enables monitoring of TCEM in immunoglobulins as an indicator of the T cell repertoire is those patients undergoing chemotherapy or radiotherapy to ablate their autologous repertoires and replace or re-seed them by transplant. In one embodiment the invention allows monitoring of TCEM patterns in immunoglobulins as an indication of the post intervention restoration of the repertoires.

In addition to allowing monitoring the T cell repertoires by analyzing TCEM patterns in the immunoglobulinome and B cells and T cell receptors in subjects iatrogenically exposed to radiation, the invention enables the monitoring of TCEM patterns and hence T cell repertoires in those individuals exposed to radiation in other settings. In some embodiments this includes individuals exposed to radiation in their workplace. In some instances, this includes individuals undergoing extended space flight. In yet other embodiments the individual is exposed through accident. In yet further embodiments the exposure of the individual who is monitored may be the result of a hostile use of radionuclides or nuclear weapons. In some particular embodiments the use of the invention enables the design of interventions to restore the T cell repertoires through development of countermeasures to be applied before or following such exposures and the monitoring of the change in the T cell repertoire following radiation exposure and interventions to correct the repertoires.

### Tissue Epitope Repertoires

The initial trigger for neoplasia is a genetic mutation, and usually many mutations, however the outcome of neoplasia is a function of how the immune system recognizes and responds to the neoepitopes resulting from the mutations. The present invention enables the characterization of patterns of neoepitopes arising in a neoplastic tissue as the result of mutations in the genes encoding multiple proteins. Hence, the pattern of TCEM and groove exposed motifs derived from the proteins in a neoplastic tissue as compared to a paired normal tissue from the same subject will identify which group of T cell targets may be best suited to differentiate neoplastic from normal tissue, through exposure of TCEM to T cells or change in the duration or frequency of exposure through changes in the dwell time in the MHC groove. In some embodiments, therefore, the invention enables the characterization and comparison of the TCEM repertoire of neoplastic and normal tissues. In further embodiments the groove exposed motif repertoires of such tissues are characterized and compared.

In tumor biopsies sequencing of proteins identifies mutations which may be critical to determining how the immune system responds to the tumor. By identifying amino acid motifs in those epitopes which are changed (neoepitopes) we can compare them to the patterns of frequency of motifs in the normal human proteome and immunoglobulinome. In some embodiments this includes identifying TCEM comprising the mutated amino acids and determining if they are common or rare findings in the two normal repertoire of the reference human proteome or immunoglobulinome or the non-mutated proteome of the affected individual. Determining how the neoepitopes compare with the frequency of occurrence in these normal repertoires can be used to select neoepitopoes most likely to elicit a antitumor response. Commonly occurring TCEM may lead to immune evasion and rare motifs may result in a more upregulated cytotoxic immune response.

### The TCEM Patterns in the Microbiome Repertoire

The human body is host to a vast commensal microbiome which occupies the gastrointestinal tract, skin, and oral, upper respiratory and urogenital mucosae. It has been estimated that trillions of bacteria of up to 1000 different species are present in the gastrointestinal tract of healthy individuals with different communities of the bacteria at different locations in the gastrointestinal tract providing a number of benefits including digestion, nutritional, neuroendocrine and immunological [9]. The diversity of bacteria provides a rich source of TCEM which stimulate and ensure clonal diversity of the T cells that engage them, either directly or following antibody opsonization or processing by antigen presenting cells. The human commensal microbiota also includes organisms other than bacteria, including helminths, protozoal parasites, fungi and viruses which may also contribute the TCEM diversity in the antigens to which the immune system is exposed. It is recognized that changes in the microbiome may be associated with disease conditions and in differential responses to therapeutic interventions. [10, 11]. It has been noted that individuals carrying a burden of gastrointestinal parasites are less prone to allergies and that administration of anthelmintics causes renewed sensitivity to allergens and other inflammatory conditions [12, 13]. In yet further embodiments the TCEM repertoire patterns in probiotic bacteria demonstrate differences from the normal microbiome of healthy or diseased individuals and allows characterization of which species will provide a more proinflammatory or immune suppressive repertoire of T cell stimulation.

In particular it is recognized that the outcome of cancer immunotherapies may be affected by the microbiome of the subject treated [14-17]. Microbiome composition has been linked to several inflammatory diseases such as ulcerative colitis [9, 18-20] and in allergies and asthma [21-23]. In yet other instances the composition of the gastrointestinal microbiome has been linked to obesity and weight loss [19, 24-28]. It has also been reported that the composition of the gastrointestinal microbiome may be linked to mental disease including depression [29]. Gastrointestinal microbiome balance may determine the susceptibility to pathogenic infections [30, 31].

In some embodiments of the present invention, the analysis of patterns of TCEM in the proteomes of bacterial species allows differentiation of the TCEM repertoire patterns in the proteomes of those bacterial species which are present in individuals responding vs non responding to immunotherapeutic interventions. In yet other embodiments the analysis of patterns of TCEM in the proteomes of bacterial species allows differentiation of patterns of the TCEM repertoire associated with obesity, inflammatory, autoimmune diseases and mental disease including but not limited to depression. In yet other embodiments the pattern of TCEM in the microbiome may be an indicator of the conditions which predispose to secondary infection by a virus, bacteria or parasite. In one particular embodiment the pattern of TCEM in the microbiome of the urogenital tract may characterize susceptibility to human papillomavirus infection. As microbiome research continues to expand additional examples will emerge in which the TCEM pattern in the microbiome is indicative of a disease condition or susceptibility or the recovery therefrom and thus the above examples are not considered limiting.

In yet further embodiments of the present invention the characterization of microbiome repertoires of TCEM allows the selection of species to favor the desired outcome of administration of a corrective bacteria to add to the microbiome and modulate the diversity of the TCEM pattern. In additional embodiments the invention enables analysis of an individual's microbiome prior to immunotherapy to evaluate the likelihood of response to therapy and to enable intervention to modulate said microbiome prior to therapy. In yet further embodiments the variation of the microbiome TCEM repertoires following intervention may be monitored. Although the preceding comments apply to bacterial constituents of the microbiome, a similar approach to the virome and parasitome is likewise enabled.

Probiotics are bacterial cultures added to food or otherwise delivered orally as a dietary supplement and which are intended to correct microbiome imbalances or provide other benefits [30, 32-36]. The present invention enables the characterization of probiotic bacteria and the contribution they make to the immune repertoire.

Reference databases of TCEM frequencies in the human proteome and immunoglobulinome have already been established as previously described in PCT/US2015/039969, incorporated by reference herein in its entirety. The immunoglobulin variable region database has been expanded to comprise over 40 million sequences and the frequency of all 3.2 million possible pentameric motifs in each recognition pattern has now been determined. In addition reference databases of the human proteome, certain pathogenic bacteria and normal gastrointestinal microbiome constituents have been determined as described in Bremel and Homan, Frontiers in Immunology, 2015 [5].

A critical feature of these databases is that they establish the frequency distribution of occurrence of each TCEM, differentiating those which are very common and likely to engender a large cognate T cell clonotype population versus those TCEM which are rare and for which cognate T cells are thus rare. The frequency of occurrence when combined with binding is an important determinant of whether a motif will result in stimulation or suppression.

### TCEM Motif Patterns in Pathogens

Just as the patterns of TCEM in the proteomes of microbiome organisms may indicate the contribution that certain bacteria in the microbiome make to the immune priming, so too the patterns of TCEM in proteomes of pathogens may provide indications of their ability to evade the immune response or to upregulate or down regulate the immune response. In some embodiments the pathogens are bacteria; in others they are viruses, in yet others they are fungi and in some embodiments they are parasites. While such TCEM patterns have been reported for some known pathogens [4, 37] they may also provide a basis for differentiating pathogens, or predicting the impact of an emerging pathogen.

### TCEM Patterns in Allergens

Analysis of allergens demonstrates a frequency pattern of TCEM that is highly distinct from the human proteome. Allergens comprise a high content of TCEM motifs which are extremely rare in the human proteome and immunoglobulinome. How or why this pattern is linked to the development of IgE responses and a hypersensitivity reaction is not known at this time. The frequency distribution features of allergens are nevertheless sufficiently distinct to prompt caution when proteins or peptides with such patterns are seen in environmental proteins or are generated in synthetic polypeptides or pharmaceutical products.

### Immune Cellular Repertoires

B cells and T cells are among the primary effector cells of the adaptive immune system. Both have cell surface receptors that enable them to carry out their functions. Starting with a germline genetic sequence, both types of cell have the ability to undergo a genetic diversification process to produce a repertoire of millions of genetically unique clonotypes, each having different receptor recognition. T cells recognize antigens on cognate antigen presenting cells causing the T cells to be activated and divide to expand the particular population. B cells also represent one type of antigen presenting cell. When B cells bind an antigen with their receptor fragments of the antigen molecule are processed with the cells and are presented on the surface to as a peptide-MHC complex to cognate T cells. By this process T cells thus provide a helper function to B cells and stimulate B cells to divide and undergo further somatic hypermutation. The hypermutation process reiteratively optimizes the receptor binding activity of the B cell. T cells do not undergo somatic hypermutation, but only undergo the initial genetic diversification. In both cases, B and T cells, each individual person develops a unique repertoire of cell clonotypes and numbers of cells within each clonotype, that is conditioned by the individual's exposure to antigens and other factors affecting the rate of replacement of each clonotype. B and T cell repertoires are dynamic and change rapidly in response to new antigenic stimuli. As the Examples indicate, the patterns and frequency distributions within an individual's B and T cell repertoire is indicative of that individual's state of health or disease. Monitoring of the repertoire can serve as a diagnostic indicator of disease and as a means of evaluating response to a therapeutic intervention. Monitoring of the B and T cell repertoire pattern and frequency distribution is also a means of assessing a clinically healthy individual's well-being, where a balanced and clonotypically diverse repertoire is indicative of health.

The analysis of B and T cell repertoires may be approached by analyzing the sequences in the receptors and determination of patterns therein, or by analyzing the T cell exposed motifs embedded within these sequences.

The T cell receptors comprise molecules of the immunoglobulin superfamily in which diversity is generated in complementarity determining regions in a somatic mutation process similar to that in immunoglobulin variable regions. The variable regions of the T cell receptors thus also comprise a repertoire in which the unique patterns of TCEM can be characterized as potential motifs which may be enzymatically processed and bound to MHC and hence themselves recognized by T cells thereby contributing to the ecosystem of internal stimuli to the overall T cell repertoire. Hence a further embodiment of the present invention is to analyze patterns of TCEM embedded within the repertoires of TCR molecule variable regions.

### Other Cellular Repertoires

There are further instances in which it is useful to monitor cellular repertoires, and the patterns and frequencies thereof. These are situations in which multiple isoforms or variants, including, but not limited to, splice variants, of a particular protein occur. In some particular instances, the presence of splice variants and the relative frequency of such variants may be an indicator that a particular target of a drug or biopharmaceutical has been lost. One example of this is CD20, in which certain splice variants are indicative of a loss of the rituximab target [38]. The various forms of the splice variant, and the relative proportions of each, can be analyzed as a repertoire. In neoplastic tissues the mutation of one or more proteins, in some cases comprising many different mutations of each protein, generates a repertoire of different protein markers in or on the cells. The change in diversity and frequency is an indicator of mutagenesis and in some cases prognosis which can be analyzed as a cellular repertoire. As noted above, cellular repertoires also include those repertoires of cells found in neoplastic tissue and sampled by biopsy. These are additional examples of cell repertoires and are considered non limiting.

### Applications of Frequency Pattern Analysis of TCEM and Clonotypic Repertoires in Guiding and Monitoring Immunomodulatory Interventions

The increasing facility of deep sequencing has led to sequencing and accumulation of repertoires of B and T cell receptors (BCR and TCR) of patients undergoing interventions such as immunotherapy, chemotherapy and transplantation, including homologous cell transplant, as well as patients suffering from a variety of pathologies, including cancers, hematologic pathologies, autoimmunity and other conditions. For BCR sequencing of such repertoires typically has spanned the regions of somatic hypermutation as well as attachment of the somatically mutated regions to sequences of genomic origin. Sequencing is typically done on a relatively small volume of blood (a few ml) or a small biopsy and results in the accumulation of many hundreds of thousands or millions of sequences for each patient. These samplings and sequencings are often done at multiple time points as the course of the disease or intervention is monitored. The generation of more and more “big data” as a result of the facility of sequencing creates a challenge in translating this into actionable information. There is therefore an urgent need for those in the field to be able to analyze the resultant large datasets of sequences in order to be able to identify and monitor characteristic patterns associated with such diseases or interventions and their progression over time. In some particular cases it may be desirable to track the change in repertoires as a companion diagnostic to an intervention. Said intervention may include but is not limited to stem cell transplant, radiation, chemotherapy, vaccination, checkpoint inhibitors, or other immunotherapies. In yet other instances the routine monitoring of B and T cell repertoires provides an indicator of health and well-being and a means to provide early warning of any immune cell repertoire dysbiosis or disequilibrium.

### Diagnostic Applications Leading to Selection of Immunomodulatory Interventions

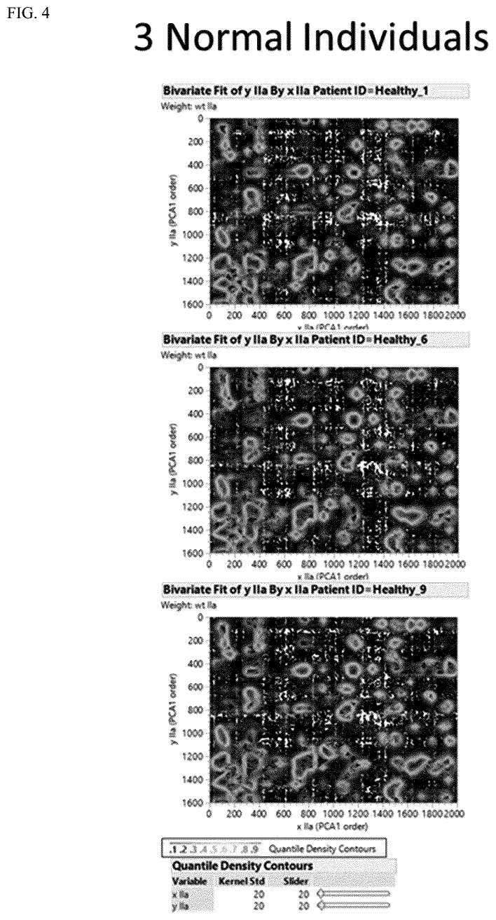

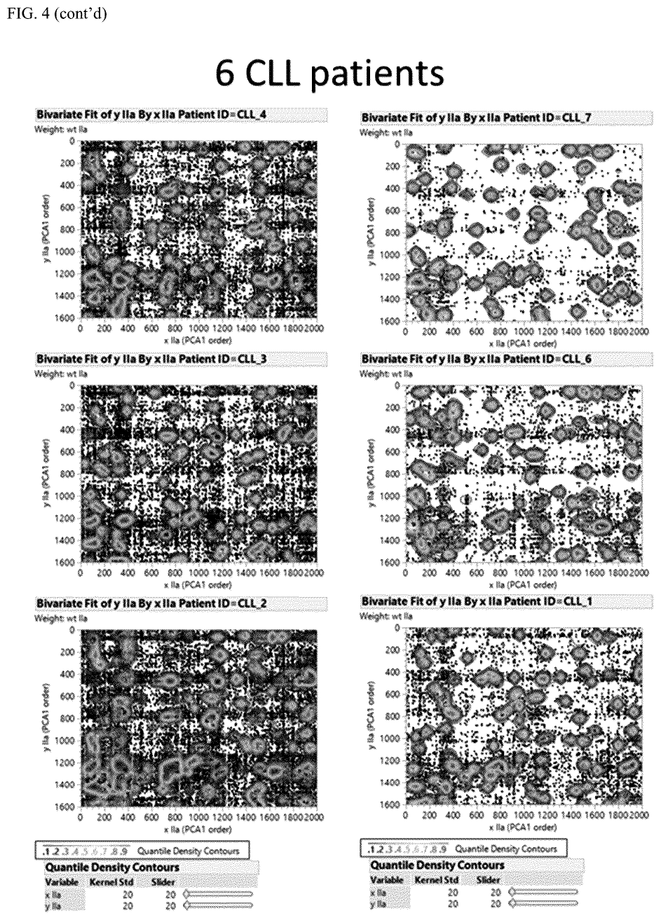

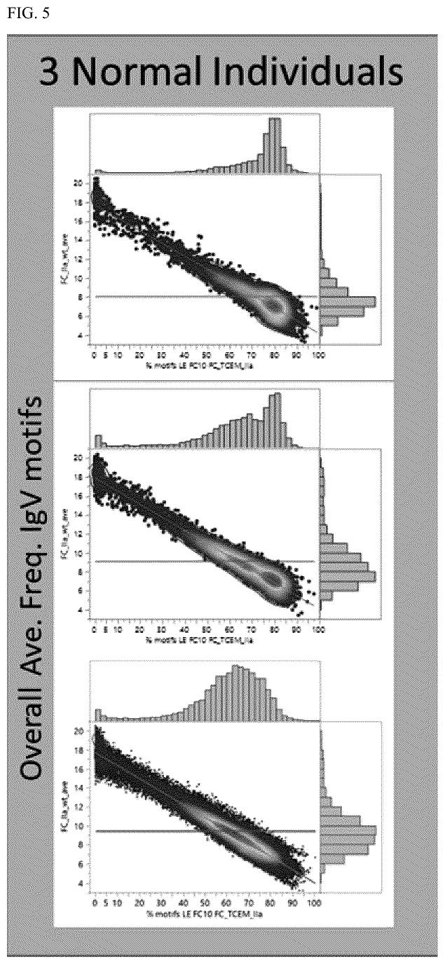

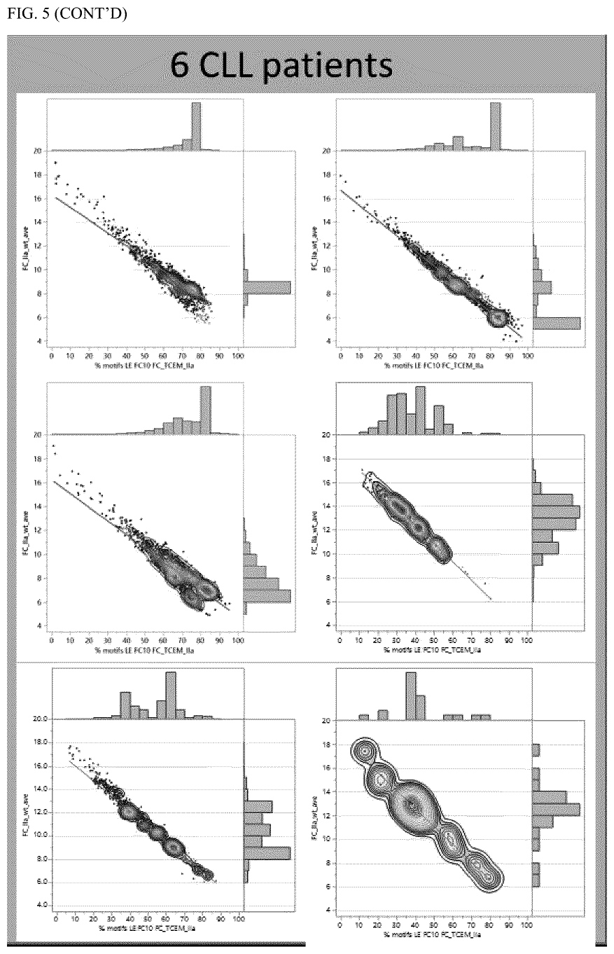

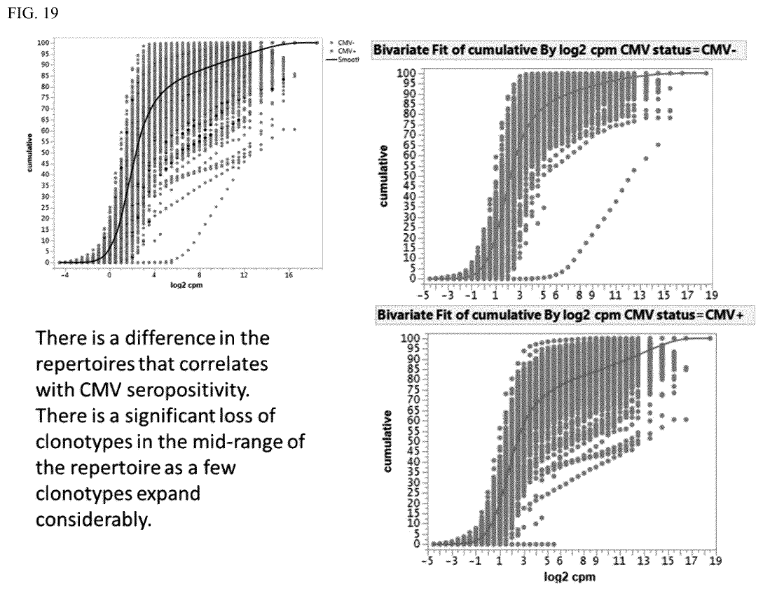

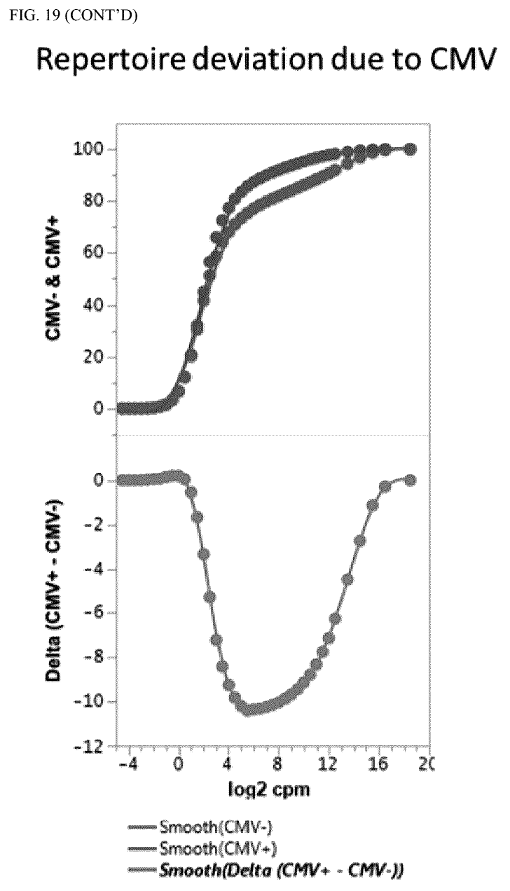

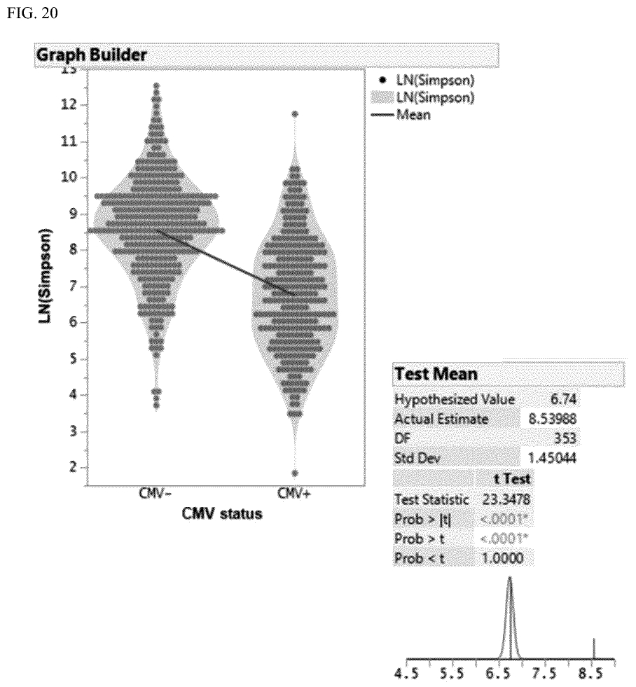

As shown in Example 2, profiling the pattern of B and T cell repertoires, either via analysis of the TCEM frequency patterns or the clonotypic frequency patterns can demonstrate patterns diagnostic of, or indicative of, certain hematologic cancers, including but not limited to leukemias and lymphomas (as shown in FIGS. 4-5), autoimmune diseases, including but not limited to those listed elsewhere in this Description of the Invention, and infectious diseases including but not limited to Epstein Barr virus and cytomegalovirus infections as shown in Example 7 and FIGS. 19-20. In one embodiment therefore, an aberrant frequency pattern may serve as an indicator for selecting chemotherapy or radiation to ablate a particular cancerous cell type, or to direct a CART or a targeted cytotoxic intervention to an excessive T cell clonal population targeting and stimulated by a particular TCEM or group of TCEMs. In yet other instances it may indicate an intervention to rebalance the T cell repertoire in a chronic disease, including but not limited to administration of IVIG, microbiome modification or immunomodulatory dietary supplements.

### Preparation for Immunomodulatory Intervention

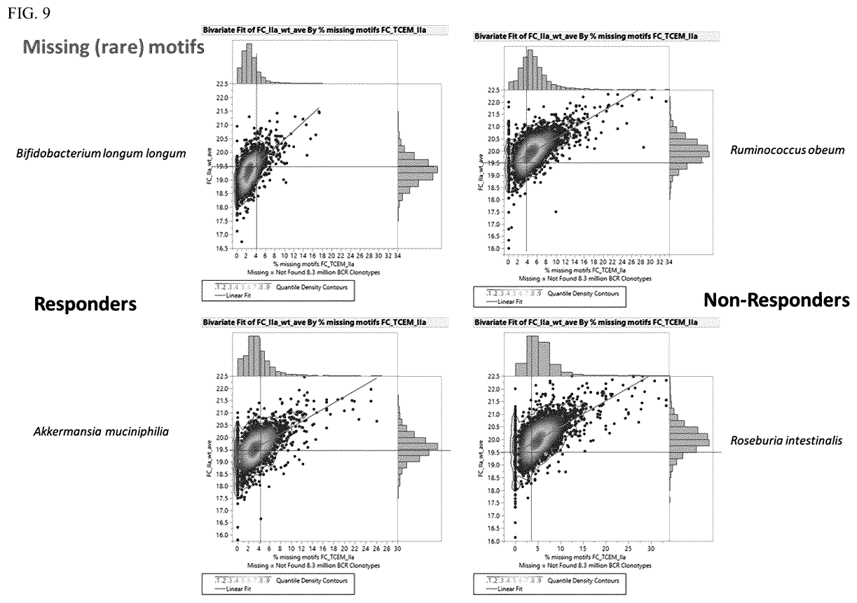

Checkpoint inhibitors, including but not limited to PD and PD-ligand blockade and CTLA4 blockade, have shown remarkable success in some cancer patients. However, the outcome is unpredictable and response rates are still relatively low [39]. There is a recognized need for better predictive markers for the suitability of checkpoint inhibitors. This includes understanding the mutational load and diversity of the tumor [40-42]. In one embodiment the present invention provides a method to increase the probability of successful treatment with checkpoint inhibitors. Checkpoint inhibitors function to prevent downregulation or shutoff of T cell responses, effectively unleashing T cells to actively target those T cell exposed motifs cognate to their receptors. However, such checkpoint inhibitors do not expand the repertoire with additional T cell specificities. Therefore, only those T cell receptor specificities present at the time of checkpoint inhibitor treatment will be available to act against the desired epitope targets. In one embodiment therefore, application of the present invention enables direct and indirect assessment of the diversity of T cells in a subject's repertoire prior to such treatment. Assessment of T cell repertoire diversity, by TCEM analysis or clonotypic analysis, provides a direct indicator of the breadth of epitope diversity which will be targeted by T cells unleashed by checkpoint blockade. B cell repertoire diversity, as measured by TCEM diversity of the immunoglobulinome or by clonotypic diversity, is an indirect indicator of T cell diversity, as B cells presenting peptides derived from endogenous immunoglobulins provide stimulation to maintain T cell repertoire diversity [8, 43]. Individuals with a broad diversity of T cell repertoire are more likely to carry T cells which are specific to, and will target, the TCEM in a particular tumor. Conversely, patients with a narrow T cell repertoire are less likely to have T cells of the correct specificity to act on that tumor. Based on an assessment of a subject's T cell repertoire prior to checkpoint inhibitor treatment, it may be determined that an intervention is needed to broaden the T cell repertoire before a checkpoint inhibitor is administered. In some cases such and intervention may be the administration of a drug or biopharmaceutical stimulating B or T cell replication, including but not limited to interleukin 2 interleukin 12, and GM-CSF, in other embodiments it may be achieved by administration of intravenous immunoglobulin (IVIG) to provide a diversity of T cell stimulation by exposure to a diversity of TCEM in immunoglobulin variable regions. In yet other embodiments, increased T cell repertoire diversity may be stimulated by oral administration of a dietary supplement comprising proteins and peptides containing diverse TCEM. One particular intervention which may be selected based on prior TCEM analysis of the T cell repertoire, is administration of oral immunoglobulin of bovine or other species origin, for instance derived from milk (See, e.g., US Pat. Publ. No. 20180221474A1 which is incorporated by reference herein its entirety). In another embodiment the T cell repertoire may be expanded by manipulating the gastrointestinal microbiome to expand the diversity of T cell stimulation, through administration of probiotics or bacterial cultures to alter the microbiome and expand the diversity of TCEM it contains which can stimulate T cells and expand the repertoire. In another embodiment, the subject's gastrointestinal microbiome may be analyzed prior to checkpoint or other immunotherapy to determine the diversity of T cell stimulation it provided by the particular microbiome of the subject, as evidenced by the pattern of TCEM contained in the microbiome proteome. A determination may then be made to manipulate the microbiome by addition of bacteria which have a broader TCEM diversity (see Example 4 and FIGS. 9-13) in order to expand the T cell repertoire it stimulates. In another particular embodiment addressing the particular instance of a neoplasia in which target epitopes arising from mutants, or from unmutated tumor associated antigens are identified, the subject may be vaccinated using a personally selected array of neoantigens corresponding to those target epitopes prior to checkpoint inhibitor treatment. In each of these cases the repertoire TCEM diversity may be analyzed before and after the intervention intended to modify it, as well as after immunotherapy.

### Application of Analysis Following Radiation, Chemotherapy and B and T Cell Transplant

The optimal status of a healthy subject exists when that subject has a balanced and diverse T cell repertoire providing T cells of specificities cognate for TCEM in all incoming challenges. In interventions which ablate the T cell and B cell repertoires, as is the case in treatment of cancers with radiation or chemotherapy, it is desirable to restore the T cell repertoire to near normal. In some instances, radiation and chemotherapy may be directed primarily to other cell populations, but diminish B and T cell populations as a side effect. In cases where radiation or chemotherapy or followed by B or T cell stem cell transplant it is similarly desirable to rapidly restore the repertoire to near normal diversity. Furthermore, monitoring the diversity patterns of the T and B cell repertoires by analyzing TCEM patterns or clonotypic frequency and diversity patterns provides a prognostic indicator as shown in Examples 8 and 11, and may guide the application of additional interventions as noted above for checkpoint inhibitors, including but not limited to B and T cell stimulants, IVIG or oral supplements and microbiome modifiers. Paucity of T and B cell diversity may also indicate vulnerability to infection which may guide the need for additional supportive therapy in such transplant patients.

In addition to the monitoring of a subject who has undergone medical radiation therapy, another embodiment is the management of subjects who have been accidentally exposed to ionizing radiation. Chronic radiation sickness is characterized by damage to immune cells and their progenitors and an acceleration of immune senescence process [44]. Following such a massive destruction of B and T cell populations, reconstitution of the repertoires is needed to reestablish self vs host discrimination and defense against infections. Currently drugs such as GM-CSF and IL12 are offered as a means to stimulate T cell proliferation [45, 46]. However, these do so without regard to the normal frequency patterns which are stimulated by presentation of peptides, and their TCEM, derived from immunoglobulins. In one particular embodiment therefore the B and T cell repertoire analysis of an individual subject who has undergone whole body radiation and who shows a loss of diversity in said repertoire, may indicate the desirability for an intervention to restore the repertoire by means of IVIG. In an alternative intervention dietary supplementation may be provided with diverse TCEM from milk or egg immunoglobulins, or by manipulation of the microbiome to increase diversity or TCEM exposure.

### Application Following an Immunomodulatory Intervention

Immunomodulatory interventions such as CAR-T therapy, and the extended application of antibody based biopharmaceuticals may lead to imbalances in the diversity of T cell repertoire. A naturally balanced stimulation of T cells provided by TCEM within a full range of naturally arising immunoglobulin variable regions is potentially supplanted or biased by domination of the T cell epitopes present in the biopharmaceutical protein. As antibody-based biopharmaceutical drugs are now the fastest growing class of drugs, this is likely an underestimated and growing issue. In one embodiment therefore, application of analysis of the frequency patterns of TCEM and clonotypes in patients who receive long term biopharmaceutical treatment is a means of monitoring the effect of such long-term immunomodulatory intervention on the repertoires and selecting a strategy to reestablish the repertoire diversity.

### Application of Analysis as a Wellness Indicator

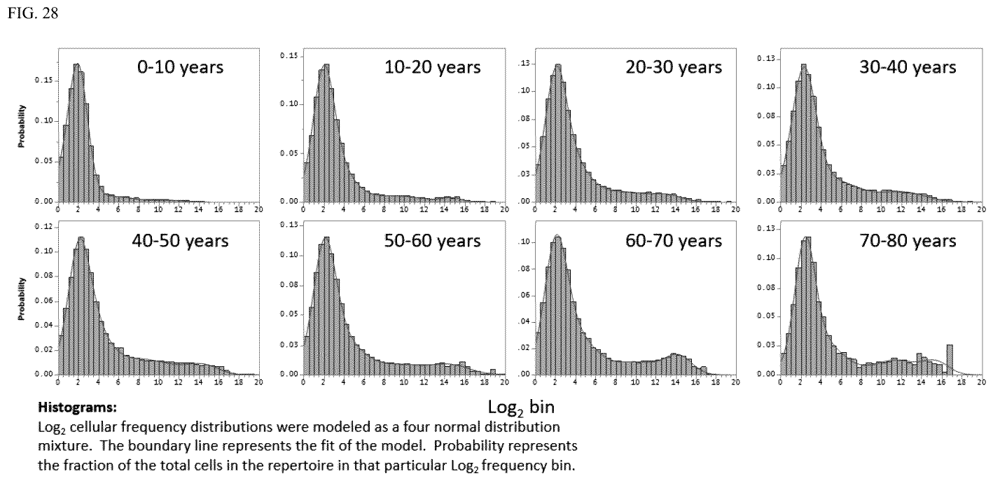

The optimal condition for a subject to resist infection, mitigate allergies, eliminate cells bearing potential neoplastic mutations, and to avoid autoimmunity is to have and to maintain a T cell repertoire that is highly diverse. A highly diverse repertoire has the greatest likelihood of having representation of T cell receptors which bind each of the possible TCEM. In one embodiment therefore, analysis of the T cell repertoire and, as an indirect indicator, analysis of the B cell repertoire, can serve as an indicator of probability of wellness or alternatively may indicate when a T cell repertoire is deficient in diversity and in need of intervention to correct the balance and increase diversity. Potential immunomodulatory interventions which may be implemented for an otherwise healthy individual include dietary modifications to provide greater diversity of stimulation of T cells in the gastrointestinal mucosa, including, but not limited to, greater dietary diversity, supplementation with highly diverse immunoglobulin variable regions, including but not limited to extracted from milk or eggs, or modification of the microbiome. In one particular embodiment, the repertoire frequency patterns of an aging individual can be an indicator of progression towards immunesenescence (as shown in FIG. 28), which can be mitigated by one of the dietary interventions indicated.

### Indicators of Tumor Diversity

Invasive tumors typically arise from an initial group of genetic mutations (trunk mutations) but each of the resultant cell clonotypes continues to mutate to generate new clonotypes (branch mutations). In some aggressive tumors such as glioblastomas, such mutations generating new clonotypes may continue throughout the lifespan of the tumor and patient, despite arrest of the tumor as the result of surgery, radiation, chemotherapy or other intervention [47]. In one embodiment therefore the profiling of the repertoire of clonotypes and the further description of these by TCEM pattern analysis can identify the emerging and continuing mutations and the rate of change of the epitopes in the tumor which may serve as targets for CAR-T or vaccine development. In another embodiment the identification of TCEM motifs in the tumor which are particularly rare (low frequency) in the human proteome can provide a means of targeting tumor and minimizing adverse off target effects.

### Patterns of Analysis in Allergens

The pattern of very rare TCEM in allergens is distinct; identification of such patterns in proteomes of microorganisms or environmental organisms can be indicative of their allergenic potential and may guide testing of individuals exposed to such organisms to determine if there is an allergic reaction and to aid in differential diagnosis of possible allergic diseases. This may prompt the implementation of interventions to counter allergic responses in an exposed subject.

### Pattern Analysis to Assist in Vaccine Design.

The application of pattern analysis to selection of motifs for inclusion in tumor neoepitope vaccines is referred to above and in Example 3 and 9. Pattern analysis may also assist in design of vaccines for infectious agents. As indicated in Example 10, pattern analysis can assist in demonstrating whether an infectious agent may itself contribute to immune suppression. Such an organism, or the proteins which contribute the common or down regulatory TCEM, would be contraindicated in developing a vaccine as inclusion of such motifs could further exacerbate immune suppression.

The present invention provides a strategy for managing and analyzing such repertoires such that characteristic patterns are revealed.

Accordingly, in some preferred embodiments, the present invention provides methods that comprise first performing frequency pattern analysis of TCEM and clonotypic repertoires for a subject (most preferably, but not limited to, a human subject) as described in detail above and in the example, using the frequency pattern analysis to determine or design an appropriate immunomodulatory intervention, and then administering the immunomodulatory intervention to the subject. In some embodiments, the subject has been previously diagnosed with a particular disease or condition. In some embodiments, where the subject has been previously diagnosed with a particular disease or condition, the frequency pattern analysis is used to further identify specific immunomodulatory interventions based on the frequency pattern analysis. In some preferred embodiments, the frequency pattern analysis is used to stratify a subject in a population of subjects so that a specific immunomodulatory intervention may be administered to the subject. In other preferred embodiments, the frequency pattern analysis is used to provide a primary diagnosis for the patient and a specific immunomodulatory intervention is administered to the patient based on the frequency pattern analysis.

As indicated above, the frequency pattern analysis of TCEM and/or clonotypic repertoires for a subject may be used to determine a specific immunomodulatory intervention that is administered to the subject.

In some preferred embodiments, the methods of the present invention comprise administering an immune checkpoint inhibitor to a subject based on the frequency pattern analysis of TCEM and/or clonotypic repertoires of the subject.

Suitable checkpoint inhibitors include, but are not limited to, antigen binding proteins that inhibit immune checkpoints, for example by PD-1, PD-L1 or CTLA-4. Suitable checkpoint inhibitors include, but are not limited to, Pembrolizumab, Nivolumab, Ipilimumab, Atezolizumab, Durvalurnab, REGN2810 (Anti-PD-1), BMS-936558 (Anti-PD-1), SIHR1210 (Anti-PD-1), KN035 (Anti-PD-L1), IBI308 (Anti-PD-1), PDR001 (Anti-PD-1), BGB-A317 (Anti-PD-1), BCD-100 (Anti-PD-1), and JS001 (Anti-PD-1). In some embodiments, the subject has or has been previously diagnosed as having a neoplasm, including without limitation, non-small cell lung cancer, small cell lung cancer, head and neck squamous cell carcinoma, renal cell carcinoma, gastric adenocarcinoma, nasopharyngeal neoplasms, urothelial carcinoma, colorectal cancer, pleural mesothelioma, TNBA, esophageal neoplasms, multiple myelona, gastric and gastroesophageal junction cancer, gastric adenocarcinoma, nelanoma, Hodgkin lymphoma, non-Hodgkin lynphoma, hepatocellular carcinoma, lung cancer, squamous cell lung carcinoma, urothelial cancer, ovarian cancer, fallopian tube cancer, peritoneal neoplasms, bladder cancer, prostate neoplasns, glioblastoma, or astrocytona.

In some preferred embodiments, the methods of the present invention comprise administering a radiation, chemotherapy or immunotherapy, B cell and/or T cell, bone marrow or cord bloodtransplant to a subject with cancer based on the frequency pattern analysis of TCEM and/or clonotypic repertoires of the subject. Exemplary chemotherapeutic and immunotherapeutic agents include, but are not limited to, alkylating agents such as procarbazine, ifosphamide, cyclophosphamide, melphalan, chlorambucil, decarbazine, busulfan, thiotepa, and the like, platinum chemotherapy agents such as cisplatin, carboplatin, oxaliplatin, Eloxatin, and the like, anti-metabolite agents such as, without limitation, Methotrexate, 5-fluorouracil (e.g., capecitabine), gemcitabine (2′-deoxy-2′,2′-difluorocytidine monohydrochloride (.beta.-isomer), Eli Lilly), 6-mercaptopurine, 6-thioguanine, fludarabine, cladribine, cytarabine, tegafur, raltitrexed, cytosine arabinoside, and the like, anthracyclines such as daunorubicin, doxorubicin, idarubicin, epirubicin, mitoxantrone, adriamycin, bleomycin, mitomycin-C, dactinomycin, mithramycin and the like, taxanes such as paclitaxel, docetaxel, Taxotere, Taxol, taxasm, 7-epipaclitaxel, t-acetyl paclitaxel, 10-desacetyl-paclitaxel, 10-desacetyl-7-epipaclitaxel, 7-xylosylpaclitaxel, 10-desacetyl-7-epipaclitaxel, 7-N—N-dimethylglycylpaclitaxel, 7-L-alanylpaclitaxel and the like, amptothecins such as irinotecan, topotecan, etoposide, vinca alkaloids (e.g., vincristine, vinblastine or vinorelbine), amsacrine, teniposide and the like, nitrosoureas such as carmustine (BCNU), lomustine (CCNU), semustine and the like, inhibitors of EGFR, antibodies to EGFRs, antisense oligomers, RNAi inhibitors and other oligomers that reduce the expression of EGFRs including without limitation, gefitinib, erlotinib (Tarceva), cetuximab (Erbitux), panitumumab (Vectibix, Amgen) lapatinib (GlaxoSmithKline), CI1033 or PD183805 or canternib (6-acrylamide-N-(3-chloro-4-fluororphenyl)-7-(3-morpholinopropo-xy)quinaz-olin-4-amine, Pfizer), and the like. Other inhibitors include PKI-166 (4-[(1R)-1-phenylethylamino]-6-(4-hydroxyphenyl)-7H-pyrrolo[2,3-d-]pyrimi-dine, Novartis), CL-387785 (N-[4-(3-bromoanilino)quinazolin-6-yl]but-2-ynamide), EKB-569 (4-(3-chloro-4-fluororanilino)-3-cyano-6-(4-dimethylaminobut2(E)-enamido)-7-ethoxyquinoline, Wyeth), lapatinib (GW2016, GlaxoSmithKline), EKB509 (Wyeth), panitumumab (ABX-EGF, Abgenix), matuzumab (EMD 72000, Merck), and the monoclonal antibody RH3 (New York Medical), small molecule inhibitors of Her2, antibodies to Her2, antisense oligomers, RNAi inhibitors and other oligomers that reduce the expression of tyrosine kinases including, without limitation, trastuzumab (Herceptin, Genentech) and the like. Other Her2/neu inhibitors include bispecific antibodies MDX-210 (FC.gamma.R1-Her2/neu) and MDX-447 (Medarex), pertuzumab (rhuMAb 2C4, Genentech), small molecule inhibitors of VEGF, antibodies to VEGF, antisense oligomers, RNAi inhibitors and other oligomers that reduce the expression of tyrosine kinases including, without limitation, bevacizumab (Avastin, Genentech). Other angiogenesis inhibitors include, without limitation, ZD6474 (AstraZeneca), BAY-43-9006, sorafenib (Nexavar, Bayer), semaxanib (SU5416, Pharmacia), SU6668 (Pharmacia), ZD4190 (N-(4-bromo-2-fluorophenyl)-6-methoxy-7-[2-(1H-1,2,3-triazol-1-yl)-ethoxy]-quinazolin-4-amine, Astra Zeneca), Zactima (ZD6474, N-(4-bromo-2-fluorophenyl)-6-methoxy-7-[2-(1H-1,2,3-triazol-1-yl)ethoxy]q-uinazolin-4-amine, Astra Zeneca), vatalanib, (PTK787, Novartis), the monoclonal antibody IMC-1C11 (Imclone) and the like, kinase inhibitors including, without limitation, compounds such as 4-(4-N benzoylamino)aniline)-6-methyoxy-7-(3-(1-morpholino)propoxy)quinazoline (ZM447439), hesperidin, AZD0530 (4-(6-chloro-2,3-methylenedioxyanilino)-7-[2-(4-methylpiperazin-1-yl)ethox-y]-5-tetrahycropyran-4-yloxyquinazoline) and tyrosine kinase inhibitors include small molecule inhibitors of tyrosine kinases, antibodies to tyrosine kinases and antisense oligomers, RNAi inhibitors and other oligomers that reduce the expression of tyrosine kinases such as CEP-701 and CEP-751 (Cephalon), imatinib mesylate, tandutinib (MLN518, Millenium), sutent (SU11248, 5-[5-fluoro-2-oxo-1,2-dihydroindol-(3Z)-ylidenemethyl]-2,4-dimethyl-1H-py-rrole-3-carboxylic acid [2-diethylaminoethyl]amide, Pfizer), midostaurin (4′-N-benzoyl staurosporine, Novartis), lefunomide (SU101) and the like, MEK inhibitors such as 2-(2-Chloro-4-iodo-phenylamino)-N-cyclopropylmethoxy-3,4-difluoro-benzami-de) (PD184352/CI-1044, Pfizer), PD198306 (Pfizer), PD98059 (2′-amino-3′-methoxyflavone), U0126 (Promega), and the like, immunotherapies, including without limitation, rituximab and other antibodies directed against CD20, Campath-1H and other antibodies directed against CD-50, epratuzmab and other antibodies directed against CD-22, galiximab and other antibodies directed against CD-80, apolizumab HU1D10 and other antibodies directed against HLA-DR, tositumomab (Bexxar) and ibritumomab (Zevalin) and the like, hormone therapies including, without limitation, antiestrogens (e.g., tamoxifen, toremifene, fulvestrant, raloxifene, droloxifene, idoxifene and the like), progestogens) e.g., megestrol acetate and the like) aromatase inhibitors (e.g., anastrozole, letrozole, exemestane, vorozole, exemestane, fadrozole, aminoglutethimide, exemestane, 1-methyl-1,4-androstadiene-3,17-dione and the like), anti-androgens (e.g., bicalutimide, nilutamide, flutamide, cyproterone acetate, and the like), luteinizing hormone releasing hormone agonist (LHRH Agonist) (e.g., goserelin, leuprolide, buserelin and the like); 5-alpha-reductase inhibitors such as finasteride, and the like, cancer vaccines including, without limitation, modified tumor cells, peptide vaccine, dendritic vaccines, viral vector vaccines, heat shock protein vaccines and the like. Other chemotherapeutic interventions include, but are not limited to, photodynamic therapy, modulators of sphingolipid metabolism, proteasome inhibitors and the like. Chemotherapy agents can include cocktails of two or more agents (e.g., KBU2046 and a chemotherapeutic and/or hormone therapeutic). In several embodiments, a chemotherapy agent is a cocktail that includes two or more alkylating agents, platinums, anti-metabolites, anthracyclines, taxanes, camptothecins, nitrosoureas, EGFR inhibitors, antibiotics, HER2/neu inhibitors, angiogenesis inhibitors, kinase inhibitors, proteaosome inhibitors, immunotherapies, hormone therapies, photodynamic therapies, cancer vaccines, sphingolipid modulators, oligomers or combinations thereof.

In some preferred embodiments, the methods of the present invention comprise administering a dietary supplement to a subject based on the frequency pattern analysis of TCEM and/or clonotypic repertoires of the subject. Suitable dietary supplements include, but are not limited to, milk immunoglobulin preparations as described in US Pat. Publ. No. 20180221474A1 which is incorporated by reference herein its entirety, fish oil and other omega-3 supplements such as krill oil or omega-3 ester concentrates, vitamin D3, ubiquinol CoQ-10, hyaluronic acid, vitamin K, vitamin K2, isoflavonoids, cathechins, gallates, quercertin, resveratrol, lycopene, curcumin, and green tea extract.

In some preferred embodiments, the methods of the present invention comprise administering a probiotic to a subject based on the frequency pattern analysis of TCEM and/or clonotypic repertoires of the subject. Suitable probiotics include, but are not limited to, supplements and other formulations comprising one or more of strains of Bifidobacterium, Lactobacillus and Saccharomyces as well as fermented food products such as yogurt, kombucha, kvass, fermented cabbage and the like.

In some preferred embodiments, the methods of the present invention comprise administering a vaccine to a subject based on the frequency pattern analysis of TCEM and/or clonotypic repertoires of the subject. In some embodiments, the methods further comprise synthesizing a vaccine with a selected representation of TCEM motifs based on the frequency pattern analysis of TCEM and/or clonotypic repertoires of the subject or modifying an existing vaccine to add or remove TCEM motifs. For example, in some embodiments, one or more TCEMs that contribute to or cause downregulation of immune response or immunosuppression are removed from the vaccine.

In some preferred embodiments, the methods of the present invention comprise administering a biopharmaceutical agent to a subject based on the frequency pattern analysis of TCEM and/or clonotypic repertoires of the subject. Suitable anti-cancer biopharmaceutical agents are described above. Additional biopharmaceutical agents include, but are not limited to, Adalimumab, Etanercept, Infliximab, Rituximab, Bevacizumab, Ranibizumab, Palivizumab, Ustekinumab and the like.

In some preferred embodiments, the methods of the present invention comprise administering a biopharmaceutical therapy to a subject and then monitoring the frequency pattern analysis of TCEM and/or clonotypic repertoires of the subject. In some preferred embodiments, the biopharmaceutical therapy utilizes a biopharmaceutical agent as described above. In other preferred embodiments, the biopharmaceutical therapy comprising administration of CAR-T cells.

### EXAMPLES

The following examples are each documented by figures. While arrays were generated for each of the three TCEM patterns: MHC I, IIA and IIB in the interests of space the Figures may show only those arrays only for one of the TCEM patterns, most commonly for TCEM IIA. All three recognition patterns resulted in similar differences in the repertoire patterns, thus the concepts and examples pertain to all TCEM recognition patterns and the inclusion of only one patters such as TCEM IIA in the figures should not be considered limiting.

### Example 1: Analysis of the Normal Repertoire in Immunoglobulin Variable Regions

Large datasets (approx. 37 million unique sequences) of normal B cell repertoires available in the public domain were used for the example analysis [48]. These datasets were divided into naïve and memory compartments that are expected to have different frequency patterns as B cells encounter antigens and selection and somatic hypermutation occurs. As a first step, nucleic acid sequences were translated to protein sequences using standard approaches. Varying numbers of unique protein sequences (clonotypes) were identified in each donor and compartment. In addition, it was noted that for clonotypes with a large number of representatives the protein sequences had been generated by different nucleic acid sequences. In total, the 37 million sequences were derived from 8.4 million clonotypes with the number of representative proteins per clonotype ranging from singletons to several thousand.

TCEM were extracted from the protein sequences using sliding windows of 9 amino acids for TCEM I and 15 amino acids for TCEM II. After this process each 9 mer and 15 mer have corresponding motifs associated with them. For each sequence a tally was created for each of the 3.2 million motif patterns and this was summarized by donor and compartment. From these tallies a clonotypic frequency was recorded for each TCEM and TCEM type. The clonotypic frequency was used as a base because it represents a unique genetic event which may be replicated many times (or not) by cell division. A log base 2 frequency classification was computed, and an integer value assigned to each motif by rounding up to the nearest integer. The scale was inverted so that the high frequency motifs had the lowest numerical values. For example a TCEM found in 50% of sequences was rounded to FC1 (FC=frequency class) and a singleton TCEM in the 8.3 million clonotypes was given a value of FC23 (½²³=8.388×10⁶). Although the somatic mutation process in principle should produce all possible pentamer TCEM some motifs were not found. These “missing” motifs were assigned a value of FC24.

Several types of graphic patterns can be used to characterize the repertoire pattern. There are differences between the naïve and memory compartments. The naïve cells emerge from the bone marrow and upon encounter with antigen begin to undergo somatic mutation. As this process ensues some clonotypes are lost and entirely and the frequency pattern will change and overtime lead to a loss of germline TCEM and an evolution towards a stable population of clonotypes. Comparison of naïve and memory repertoires allow the definition of the motifs which are uniquely found in one but not the other vs those motifs which are shared.

A total of 20⁵ TCEM can be conveniently displayed as a rectangular array of 2000×1600 elements. This makes it possible to create matrices from different biologically relevant subsets and by using computer algorithms to create patterns of TCEM that comprise the entire repertoire. Patterns are easily discerned by coloration of the numerical information as so-called “heat maps”. In addition, this type of consistent display makes it possible to readily identify pattern differences in the different biological compartments and between individuals. There are certain biological conditions where the repertoire of an individual is expected to change over time and this likewise can be displayed by doing simple arithmetic calculations on the TCEM frequency matrices.

This utility of this capability is readily seen by apparent differences between the naïve and memory compartments within individuals and by the differences between individuals. Different algorithms can be used to assign colors in the array that thereby to accentuate different features as appropriate.

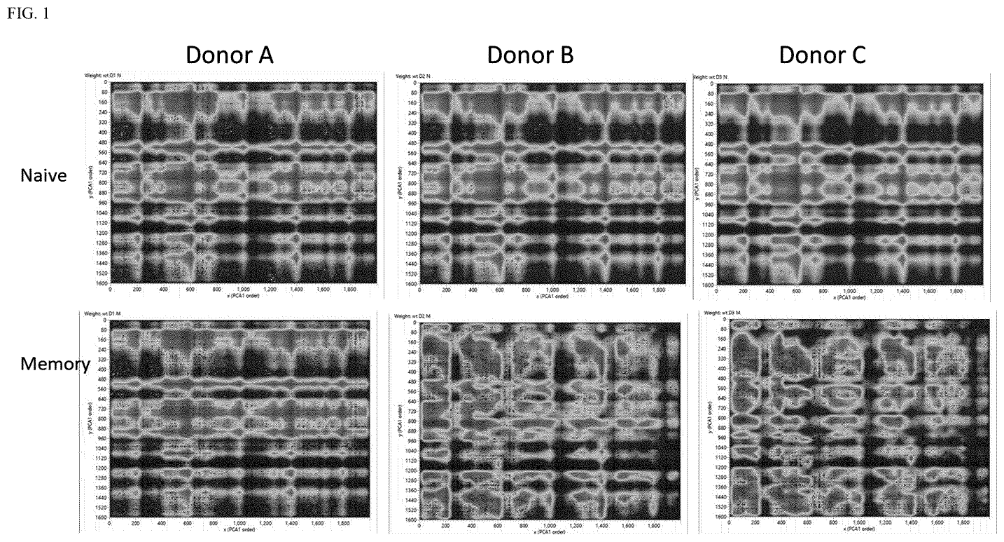

Shown in FIG. 1 is a pixel patch graphic depicting the frequency of each of each of the 3.2 million motifs in a 2000×1600 array. Patterns of motif occurrence are not random and contours are drawn based on TCEM that share motif frequency characteristics. In the patterns shown colors change at 5 percentile contour increments.

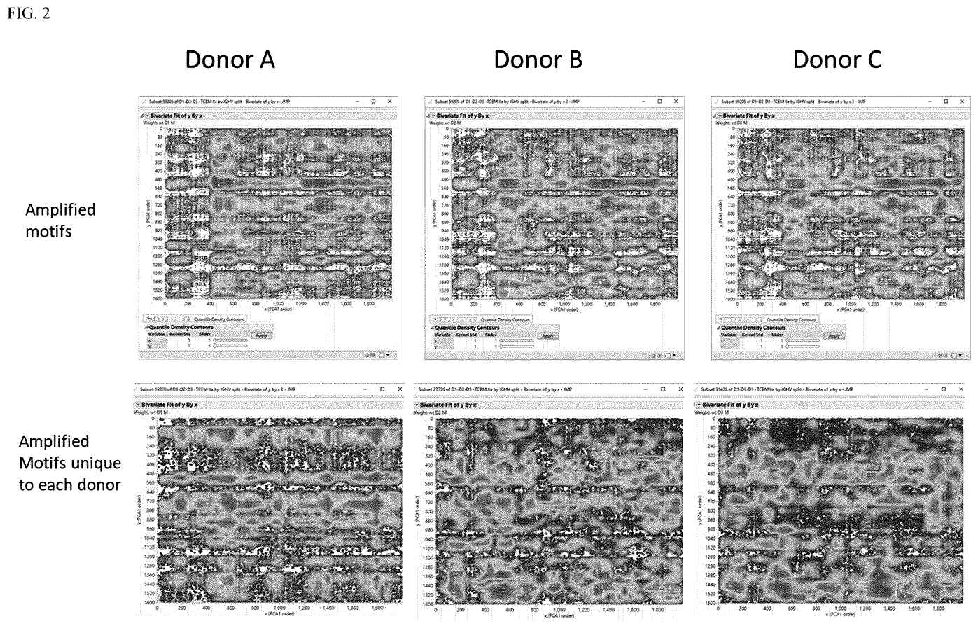

Shown in FIG. 2 is an example of a pixel patch showing the differential between two different repertoires, those of naïve and those of memory cells. In this case a simple arithmetic difference has been computed for each of the 2000×1600 elements in the matrix and then contours are applied in a similar manner to FIG. 1 but for the differences between the repertoires.

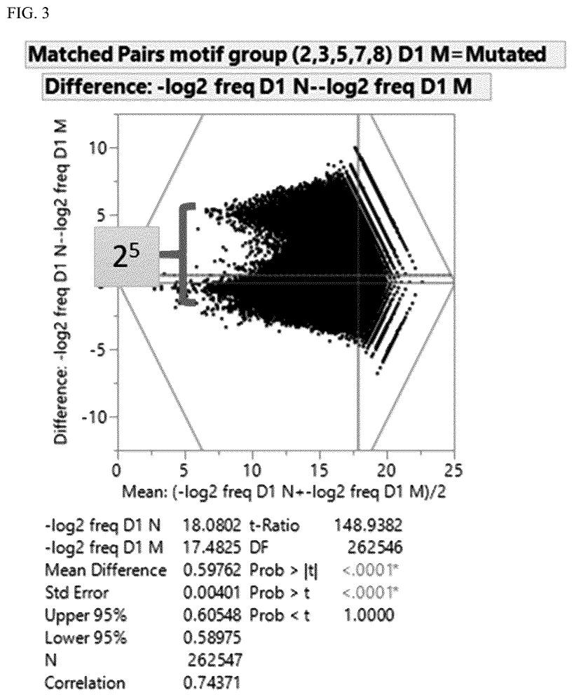

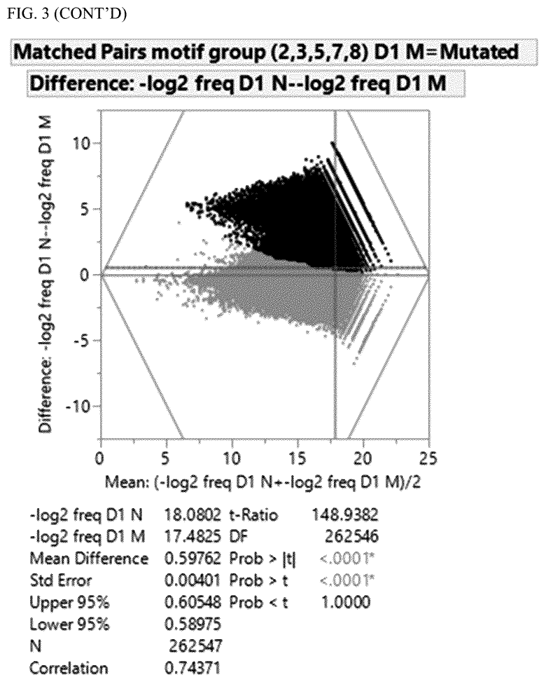

Various types of graphic are useful depending on the comparison. FIG. 3 shows the distinct way that TCEM frequencies change for virtually the entire 3.2 million patterns on the molecular evolution of naïve to memory cells.

### Example 2: Comparison of TCEM Repertoire in Multiple Chronic Lymphocytic Leukemia Patients

It is common for a B cell repertoire to undergo a change in response to an illness or due to vaccination. One of the types of illness that leads to repertoire changes is leukemia. The underlying cause of the disease may or may not be linked to the B cell receptor but a genetic mutation in an oncogene will lead to a derangement in a particular B cell clonotype and will lead to tumor growth. As a result, the TCEM repertoire of that particular clonotype will come to dominate the cell population. An example is CLL (Chronic lymphocytic leukemia). Datasets of patients with this illness are publicly available [49] and the TCEM extraction process described above was also carried out with these datasets. In CLL the mutated clonotypes grow aggressively and effectively become the dominant cell type. Because these cells have characteristic TCEM patterns these patterns and changes in the patterns are readily displayable. The changes in TCEM patterns are substantial and different types of graphic display can be used. In a normal repertoire a wide range of TCEM frequencies are seen with a weighted average frequency ranging from FC8-FC10. In CLL different repertoire clusters are seen with weighted averages over a range of frequencies. Overall, the TCEM repertoire populations tend to be skewed or be multimodal. Patients with CLL and undergoing typically have repeated periods of remission and recurrence. Graphical patterns such as these can readily be used to assess the response to treatment by repeated sampling and analysis over time.

In FIG. 4 the pixel patches of normal controls are compared to those of six CLL patients. The sparse patterns with “hot spots” indicate the dominance of a few neoplastic clones. The graphic shown in FIG. 5 can be used to display the difference between the frequency of motifs in particular clontotypes in the repertoire as it relates to the weighted average of the particular motif usage. This is particularly useful in showing the clusters of related but aberrant motif clusters in the B cell repertoire. The differences between the pattern found in the blood of CLL patients compared to normal donors is readily apparent. Monitoring the change of such graphics can provide an indicator to progression or response to intervention.

### Example 3: Neoepitope Repertoire Analysis

T cells and characteristics of their immune function are currently a focus of many different therapeutic approaches in oncology and multiple animals are being used as models for the human disease. The above examples consider the TCEM embedded within the variable region of the B cell receptor and therefore the immunoglobulin produced by the particular B cell. Thus, when these endogenous proteins are processed by endopeptidases, the MHC on the B cell will display fragments of the B cell receptor [7, 8]. Vaccines using regions of the B cell receptor have been used to effectively cause CLL remission. However, the underlying cause of the disease can be due to any number “driver” genes [50-53].

Certain breeds of dogs develop CLL which is highly similar to human disease. Genomic sequencing of the B cells in the dogs with CLL can be used to identify genomic regions outside of the BCR with neoantigens—sequences that have been generated by mutational events that will be recognized as “non-self” and thus be capable of stimulating an immunological response. In this case the focus of the analysis is on proteins that have undergone one or more mutational event(s). Synonymous mutations that do not result in an amino acid change are not important because they are identical to the normal proteins. A mutation that changes the amino acid sequence in a protein will produce a novel peptide with potentially a novel TCEM. Whether or not the TCEM actually changes will depend on the context and whether the mutation is expected to affect the binding by being in a groove exposed region or protruding to be recognized by a T cell. Depending on the amino acid change that occurs in a mutation a TCEM has the potential of interacting with a different set of cognate T cells as compared to the wild type sequence. Other TCEM changes will occur when a frameshift or splicing variant is produced. This type of mutations that have an open reading frame have a possibility of generating and number of unique amino acids and therefore multiple TCEMs until a stop codon is reached. To identify potentially useful peptides for therapeutic application it is necessary identify the TCEMs most likely to generate a cytotoxic T cell response. TCEM patterns in cellular proteins can extracted as described above for IG variable regions. In addition, the MHC binding affinity of the peptides containing the TCEM are also predicted using neural network algorithms. Peptides that do not bind to the MHC or bind with low affinity are not expected to be capable of generating a useful T cell response simply because the dwell time of the peptide in the MHC is too short and therefore the probability is low for a stimulatory cognate T cell interaction to occur. Thus, from the array of peptides in all of the proteins with mutations a subset of peptides is selected that are expected to bind with sufficient affinity such that a useful cognate T cell encounter will occur.

By knowing the MHC genotype of an individual it is possible to make predictions of the peptides that are most apt to bind to that individual's MHC molecules and thus will provoke a useful T cell response. Although the dog is a good model for human disease the MHC molecules of dogs are not the same as human MHCs. Comparisons of the potential amino acid contacts with a peptide in the binding groove of the dog MHC molecule suggest that some of the dog MHC molecules are similar to humans but others are not. It was noted that regions of the molecules with the neoantigens bound with potentially useful affinities to a number of different human MHC alleles. Given that there was a similarity between some of the human and dog alleles a strategy was devised to identify regions of the molecules where good binding was expected to a plurality of the human alleles. This process was designed to select longer peptides (>20 aa) that would be expected to be process by dog APC and converted into binding peptides that would provoke a useful T cell response. From this process about 75% of the non-synonymous mutations and frameshift mutations were predicted to be likely to produce peptides with high affinity binding and to generate useful T cell responses.

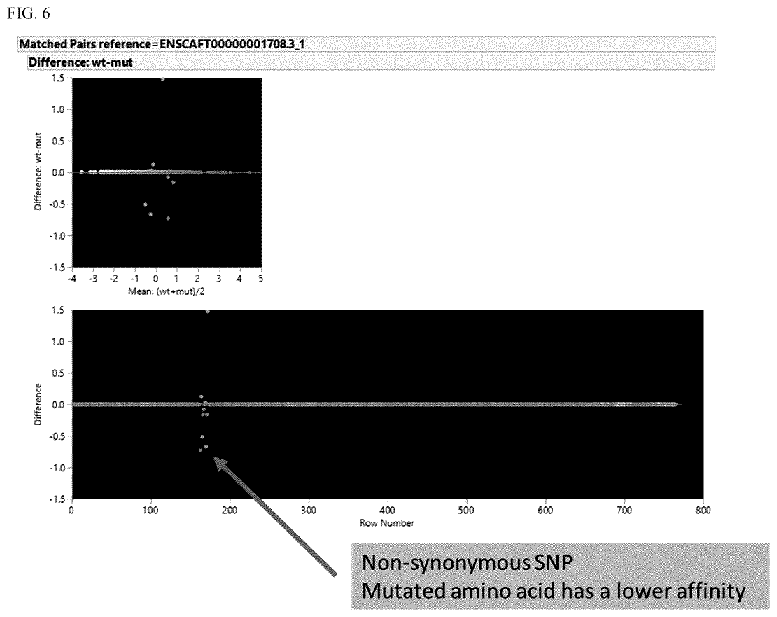

Shown in FIG. 6 is the differential motif affinity in a protein pair comprising the native (wild type) protein as compared to the same protein with a non-synonymous mutation giving rise to changes in binding affinity in the region of the mutation.

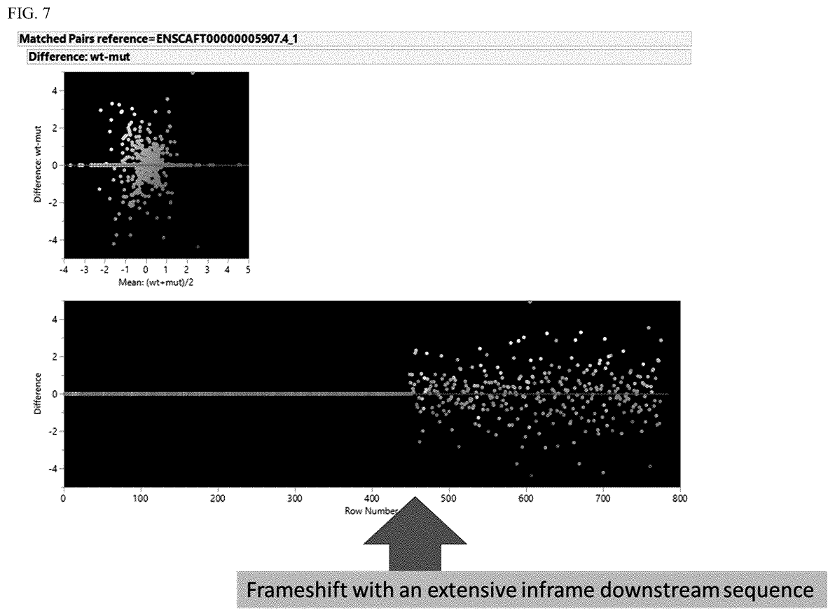

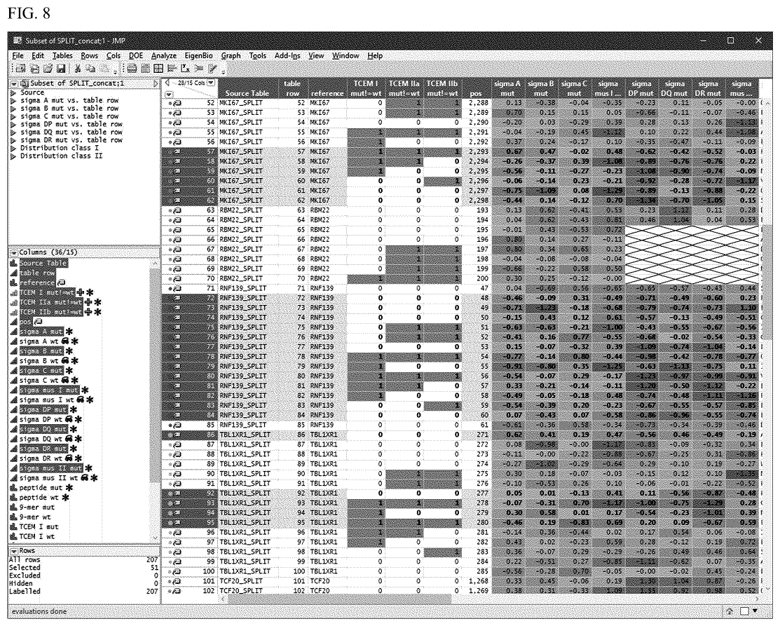

Shown in FIG. 7 is the pattern seen when a frame shift occurs giving rise to segment of considerable length where the motifs are different from the wild type sequence until a new stop codon is encountered.

Shown in FIG. 8 is an example of a protein region wherein a stretch of adjacent overlapping peptides are predicted to have high binding activity in various binding registers for a large number of human MHC alleles with the average over many alleles exceeding 1 std deviation below the mean for all the alleles under consideration.

### Example 4: Bacterial Microbiome Repertoire TCEM Patterns

The bacteria associated with response vs non response to checkpoint inhibitor therapy of various cancers has been described [14, 17, 32]. The species of bacteria associated with response vs non response [15] are shown in Table 1.

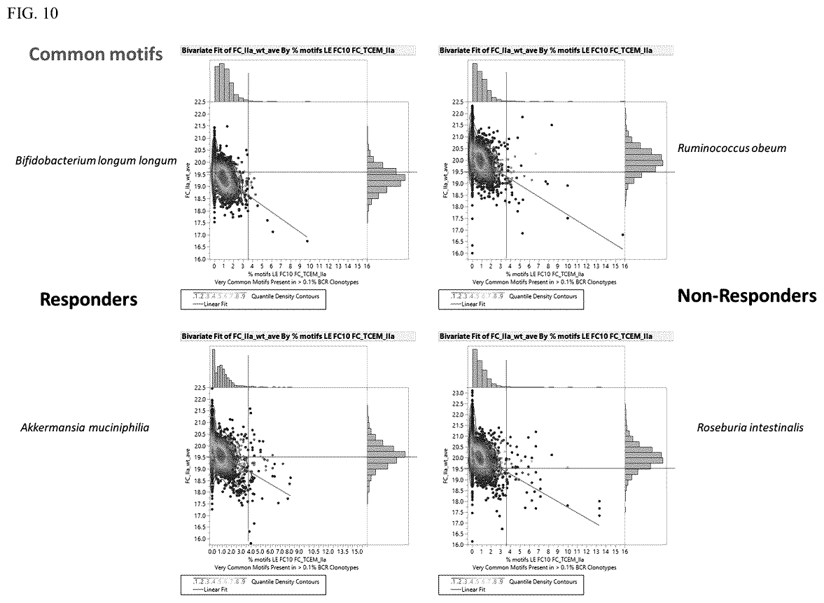

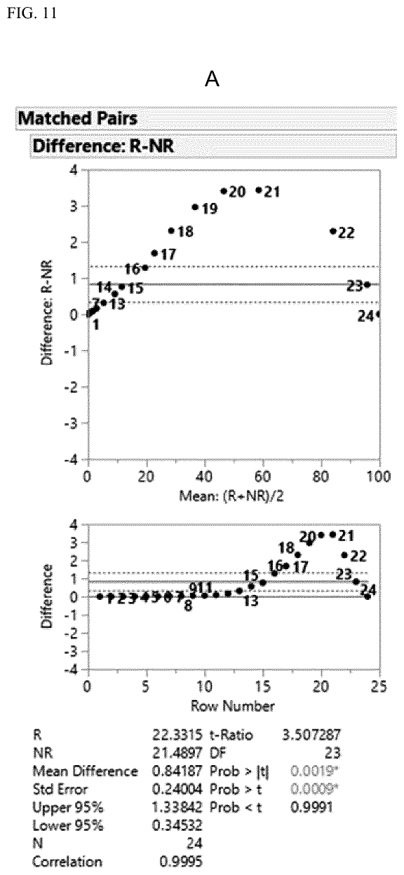

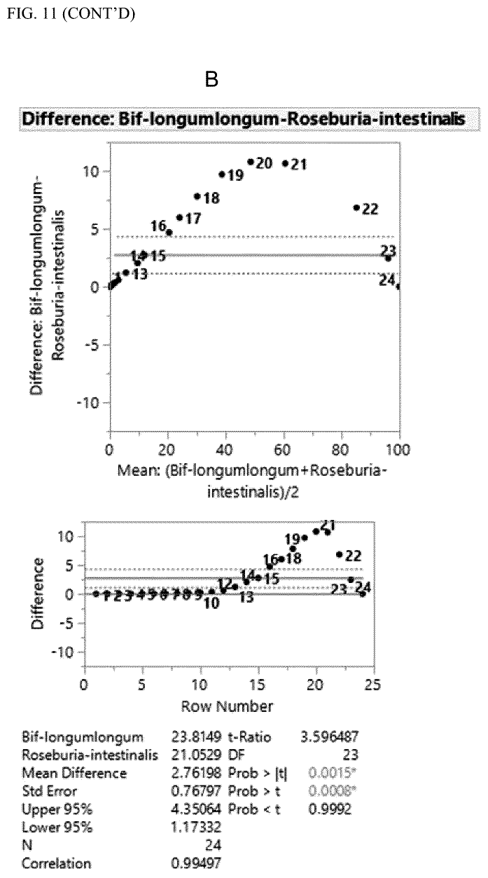

TABLE 1
Microbiome constituents identified in metastatic melanoma
patients treated with anti PD-1 check point inhibitors.

| | |
|---|---|
| Roseburia intestinalis | Non responder |
| Ruminococcus obeum | Non responder |
| Burkholderiales bacterium 1 1 47 | Non responder |
| Bacteroides intestinalis | Non responder |
| Adlercreutzia equolifaciens | Non responder |
| Holdemania filiformis | Non responder |
| Coprococcus comes | Non responder |
| Veillonella parvula | Responder |
| Enterococcus faecium | Responder |
| Collinsella aerofaciens | Responder |
| Bifidobacterium adolescentis | Responder |
| Bifidobacterium longum | Responder |
| Klebsiella pneumoniae | Responder |
| Parabacteroides merdae | Responder |
| Lactobacillus | Responder |
| Enterococcus faecalis | Responder |
| Escherichia coli | Responder |
| Escherichia unclassified | Responder |
| Bacteroides ovatus | Responder |
| Turicibacter sanguinis | Responder |
| Collinsella aerofaciens | Responder |
| Clostridium scindens | Responder |
| Clostridium nexile | Responder |
| Actinomyces graevenitzii | Responder |
| Eubacterium siraeum | Responder |
| Lachnospiraceae bacterium 7 1 58FAA | Responder |
| Bifidobacterium longum | Responder |
| Haemophilus parainfluenzae | Responder |
| Lachnospiraceae bacterium 6 1 63FAA | Responder |
| Klebsiella oxytoca | Responder |
| Campylobacter gracilis | Responder |

Where species were identified the complete proteome of each bacteria was downloaded from Patric (www.patricbrc.org), using the reference species for each. The TCEM were extracted from each protein in the proteome and processed as described above to assemble frequency distributions and pixel patch displays. FIGS. 9 to 13 show examples of the comparison of the motif frequencies in microorganisms common in patients that responded to checkpoint inhibitors as compared to those that did not respond. In FIG. 9 each point corresponds to a protein in the proteome, and is plotted according to the composite motif frequency metric in the entire sequence of the particular protein in the genome of the microorganism. The X axis is the percentage of very rare motifs in the protein and that comprise “missing” motifs in the protein that are not found in 8.3 million naive and memory BCR clonotypes. The Y axis is the weighted average of the FC (frequency class as determined by reference to an immunoglobulin variable region database) within the protein for all of the proteins in that organism. The center of mass is indicated by the contoured area. The cross-hairs superimposed are for comparative purposes. The center of mass of the non-responders is seen to be in the upper right quadrant indicated by the cross hairs. This indicates that the non-responders tend to have a greater fraction of proteins with unusual motifs (percentage of “missing”) and as a result have a higher weighted average of FC of the motifs in their proteins. By contrast proteins in the microorganism common in responders have fewer missing (extremely rare) motifs reflected in a lower FC weighted average over the entire proteome (FIG. 10). However, in FIG. 11 it is noted that species from reponders as a whole, and selected bacteria dominant in responders vs non responders have a higher content of TCEM that are in the rare frequency category FC16-23. Both bacteria from responders and non responders have representation of motifs that are common (FC1-10). Hence the bacteria in responders comprise a repertoire with higher diversity (comprising FC1-23) and ability to stimulate and maintain a diversity of T cell clones each with the potential to become effectors acting on the tumor upon application of the checkpoint inhibitor.

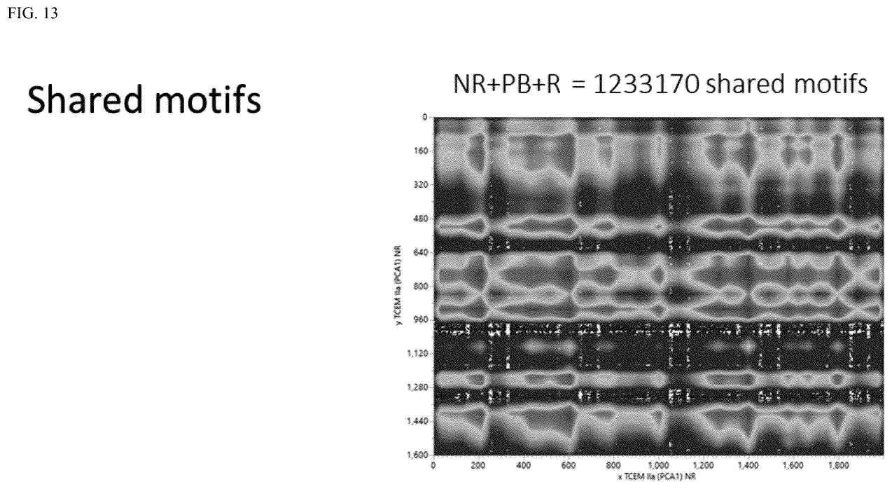

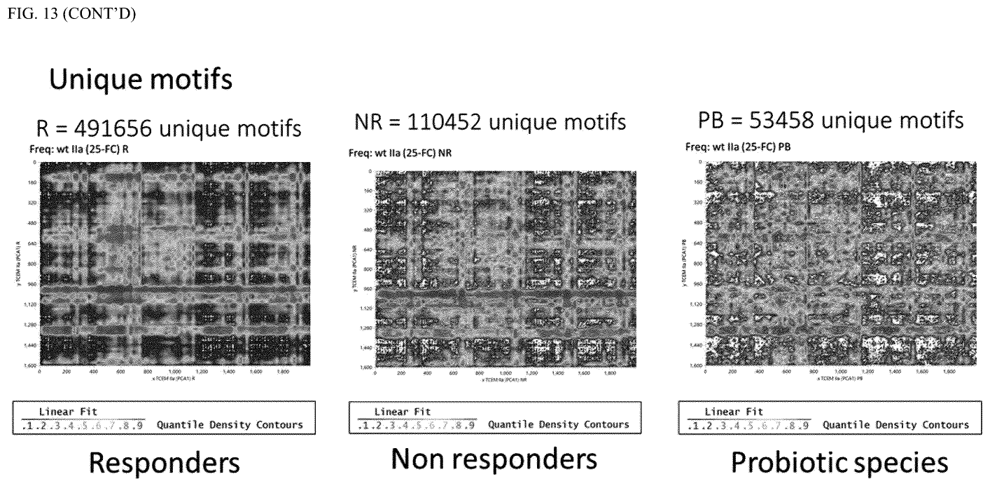

Pixel patches were then generated to examine the differences between the TCEM motifs in responder populations vs non responders and in probiotic bacteria as shown in FIG. 13. Probiotic bacterial species are shown in Table 2. Distinct differences in the overall patterns of T cell exposed motifs that are unique to the microbiome of responders vs non responders vs probiotic bacteria are noted, corresponding to the differences in TCEM content and frequency of each noted in FIGS. 9-12

TABLE 2
Probiotic species analyzed

Bifidobacterium bifidum PRL2010
Bifidobacterium infantis ATCC 15697
Bifidobacterium lactis DSM 10140
Bifidobacterium breve
Bifidobacterium Sp12-1-47B
Lactobacillus acidophilus NCFM
Lactobacillus helveticus DPC 4571
Lactobacillus rhamnosus GG ATCC 53103
Lactactobacillus reuteri

The proteomes of the probiotic bacteria were processed as above to extract TCEM and compare TCEM frequency distributions.

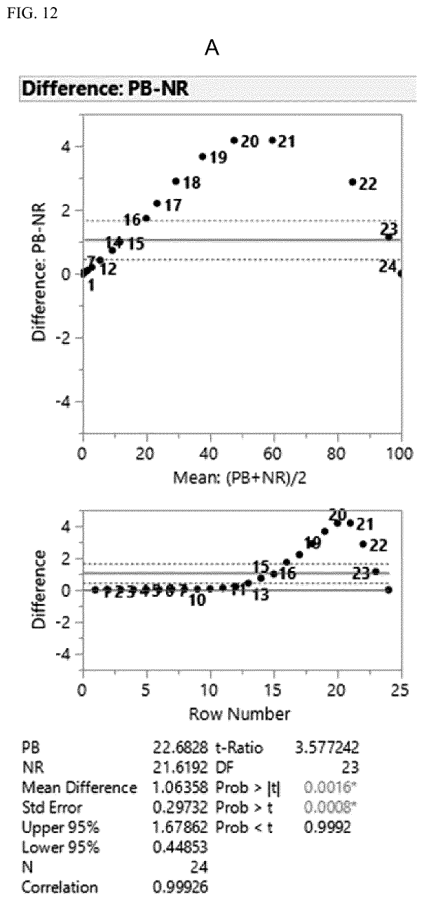

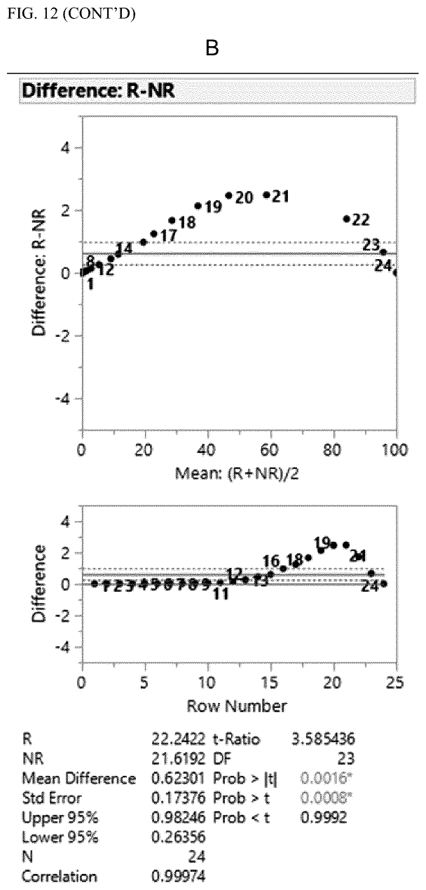

FIG. 12 shows how the probiotic bacteria as a group comprise a yet greater diversity of TCEM in FC16-23 compared to the group of bacteria from non responder cancer patients than do the responder bacteria. Hence the probiotic bacteria may offer a broader diversity of T cell stimulation.

### Example 5: Epitope Networking Arrays of T Cell Receptor Motifs

Like antigen presenting cells such as dendritic cells, T cells also display peptide fragments of proteins in MHC molecules on their surfaces. As a result, T cells will also display motifs derived from their own receptors bound as peptides in MHC, just as do B cells. These TCEM exposed in MHC will be recognized by other T cells and thus comprise a T cell:T cell collaboration network much like the T cell:B cell collaboration network. Hence both T and B cells act to complement each other via TCEM recognition in repertoire stimulation and maintenance.

The CDR3 region of the TCR is known to be the region of the molecule that interacts with TCEM presented on MHC molecules and comprises the variable component of the TCR. Thus the pentamers exposed in pMHC on the surface of T cells will be a unique signature of a particular CDR3 clonotype. The same CDR3 will be combined with different V, D and J regions in a stochastic mutation process that provides additional diversification of TCEM by combining the regions immediately flanking the CDR3 with the CDR3 itself. Analysis of the arrays of TCEM motifs and the frequency of each motif can thus provide an indicator of the diversity of the TCR population in an individual, or in a subset of the T cells in an individual subject.

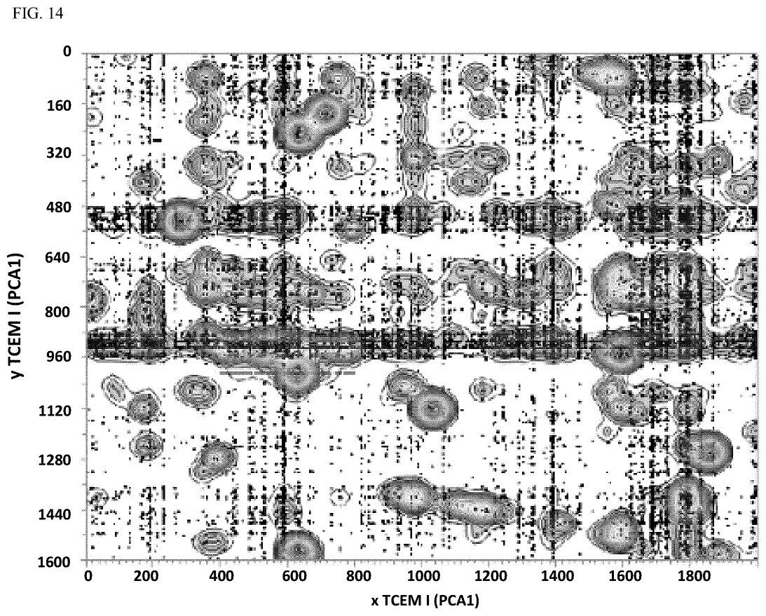

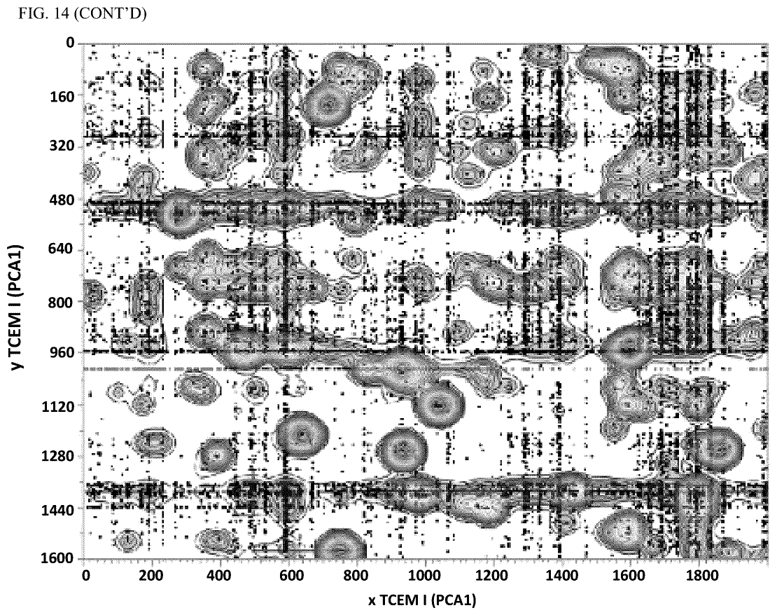

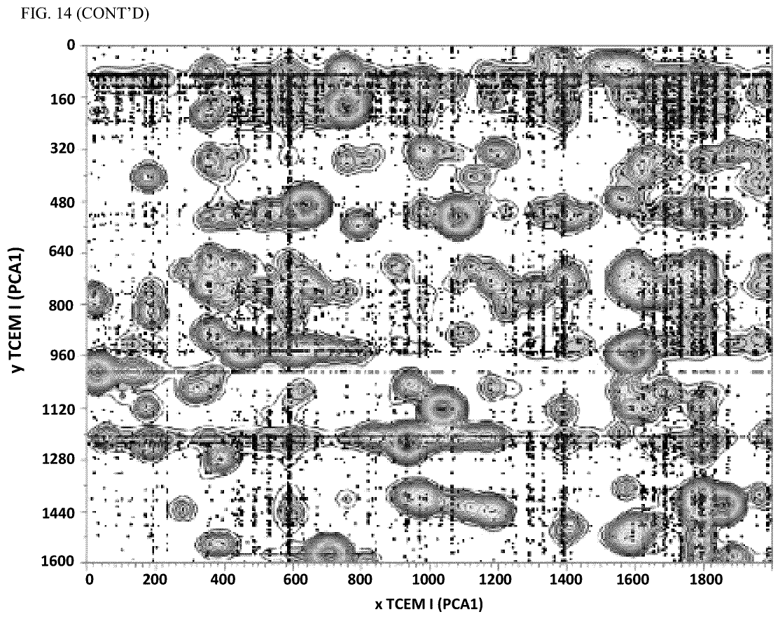

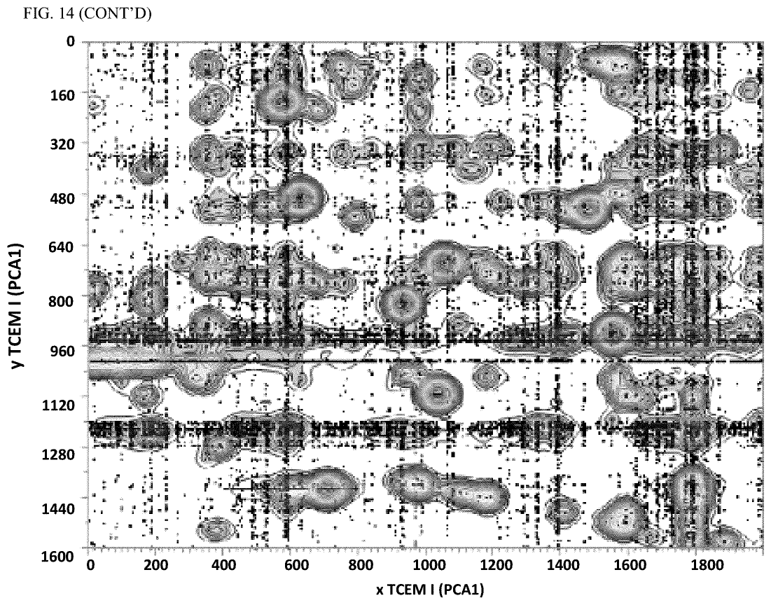

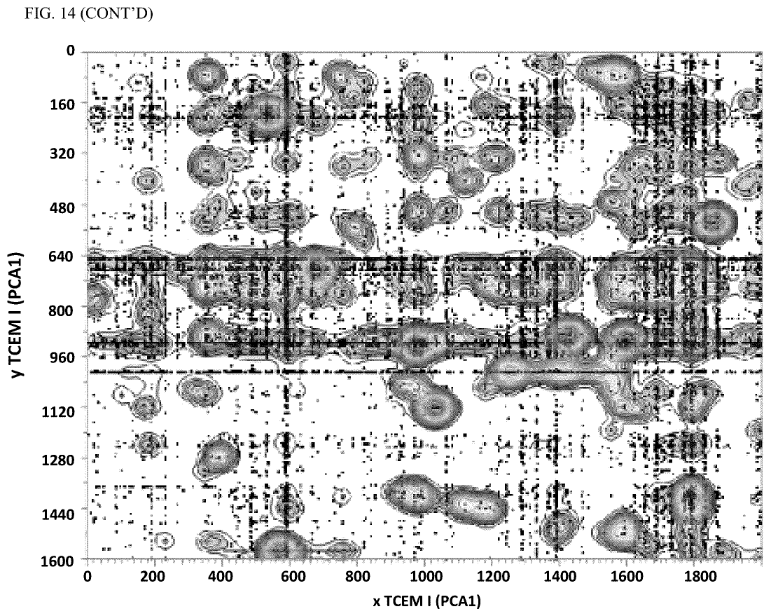

To display relevant TCEM from a T cell repertoire, TCEM are extracted from each unique T cell clonotype. For a MHC II TCEM “pixel patch” display, any 15 mer from the sequence covering CDR3 and V, J, D that contains 1 or more amino acids from the CDR3 region is included. The 15-mers thus include the flanking regions of the comprising the VD & J regions of different T cell family origins. After this process, the extracted TCEM are displayed on the standard 2000×1600 coordinate system. The patterns displayed are for the 5 most common CDR3 clonotypes for a particular TRAV family. The displays are weighted by the numbers of each clone in the repertoire a process which therefore should provide a visualization of the contribution of the clonotypes to the repertoire. By using this process one can also follow changes in the repertoire of an individual over time after a treatment that would be expected to cause changes in the repertoire such as after vaccination or stem cell transplant reconstitution. An example is shown in FIG. 14.

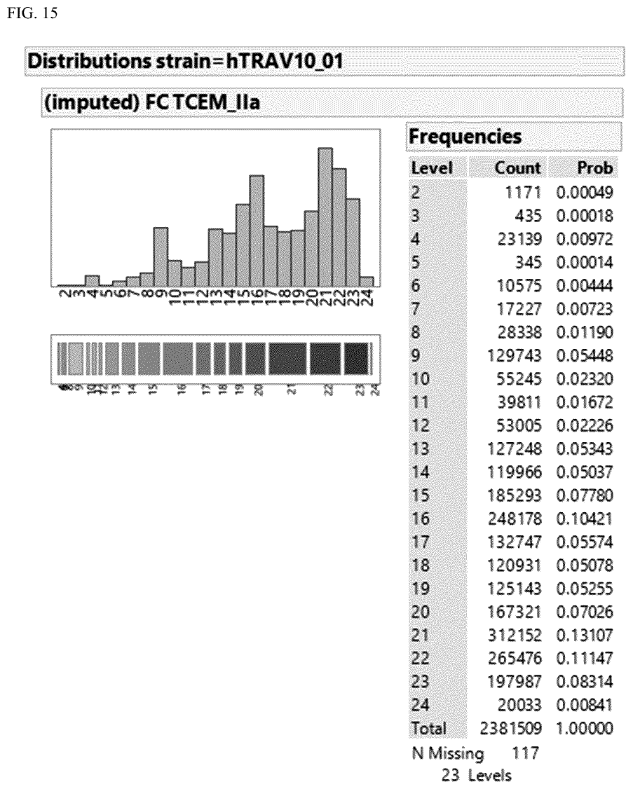

Notably the TCEM found in hTRAV are arrayed on a frequency distribution similar to that in BCR, as noted in FIG. 15, which provides an example of the frequency distribution for human TRAV subgroup 10. Similar frequency distributions are observed in hTRBV.

### Example 6: Frequency Distribution of T Cell Receptors and B Cell Receptors in Repertoires

When the probability of measuring a particular value of some quantity varies inversely as a power of that value, the quantity is said to follow a power law. This also known variously as Zipf's law or the Pareto distribution. Power laws appear widely in physics, biology, earth and planetary sciences, economics and finance, computer science, demography and the social sciences. The origin of power-law behavior has been a topic of scientific debate for more than a century. A general characteristic of a power law distribution is that the cumulative distribution histogram or rank/frequency plot are linear when plotted on log x vs log y axes [54].

T cell and B cell receptor repertoires also exhibit power law characteristics in protein sequences that have resulted from a somatic hypermutation process. This is analogous to the observations of Li in analyzing word frequencies [55]. Plots of BCR and TCR clonal frequency and abundance are similar to those described by Newman and Naumov [54, 56] with different repertoires showing very subtle changes in the cumulative distribution pattern.

Tracking of temporal changes in cell repertoires for diagnostic purposes is challenging because the number of clonal lines in any individual subject number in the tens of millions, and over time their ranks and frequencies tend to undergo exponential changes. Such changes are of biological relevance. However, even with large changes the cumulative distribution plots remain essentially linear with only very subtle changes that are statistically difficult to dissect.

The process of logarithmic binning is often used in power law analysis. Here we apply logarithmic binning to analyze the frequency of clonal cells. Based on identification of clonal cells as determined by sequences of their TCR, the clonal cells (normalized and expressed as cells per million) was placed into log 2 bins. Thus, in bin 0=2⁰=a singleton or one cell in a million with that particular TCR, whereas in bin 17 will contain clonal lines with >2¹⁷→>131,072 cells/million representatives. Importantly the unique feature of this process is that it focusses on the low frequency portion of the distribution; it is essentially an inverse of the standard cumulative distribution.

Optimally responsive T and B cell repertoires of healthy individuals will be maximally diverse, having a large percentage of cells in the low frequency/low abundance portion of the cumulative distribution plot. Conversely, repertoires with more dominant clones (sometimes with a few very dominant ones) are characteristic of diseases like lymphomas or leukemia. Thus, an effective disease intervention will result in establishment and maintenance of a pattern with greater clonal diversity. Certain diseases result in shifts in the clonal dominance patterns. Therapeutic treatments that are corrective will likewise lead to other changes in clonal diversity. The types and magnitude of changes vary considerably and can be useful diagnostic indicators. Effectively this means cell clonal diversity is sliding up and down the linear slope of the standard rank/frequency cumulative distribution plot.

Various types of patterns that can be elucidated using an inverse frequency distribution analysis.

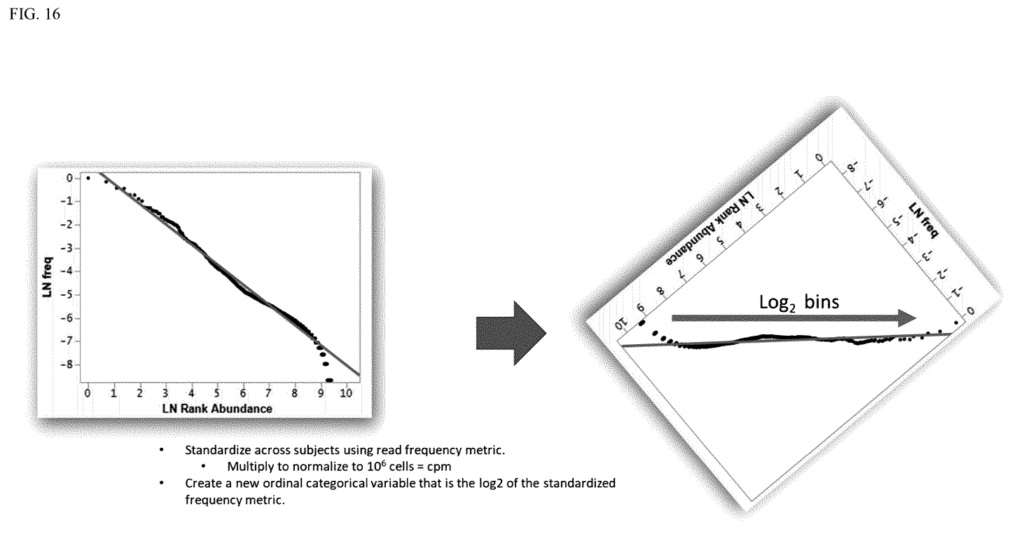

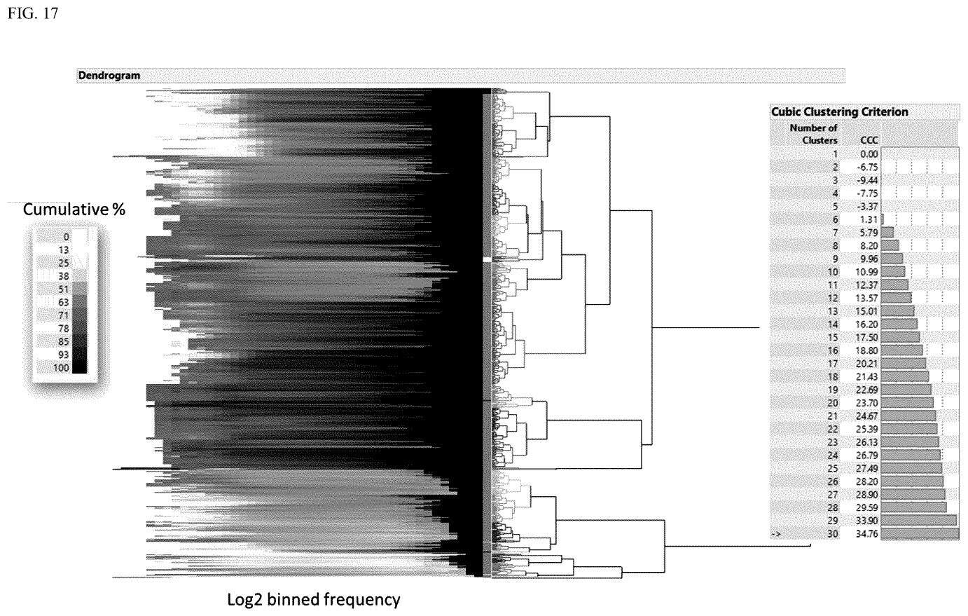

FIG. 16 illustrates the process of logarithmic binning. The shape of the clonal frequency patterns vary greatly among individual subjects. A simple power law display such as that in FIG. 16 is easy to interpret for an individual subject but becomes difficult to understand in the face of clonal expansion patterns or those of multiple individuals. Hierarchical clustering based on the clonal frequency binning pattern can be used to visualize the cellular frequencies within an individual and to compare and contrast different individuals. Subjects with a very narrow pattern repertoire (fewer clones, with higher frequencies for each) will not have the ability to respond to a wide range of challenges. A broad, highly diverse cellular population in the repertoire will have the most likelihood of being able to respond to new challenge to the homeostatic balance. FIG. 17 shows a dataset comprising the repertoires of 664 subjects segregated into 30 different subsets based on the repertoire composition.

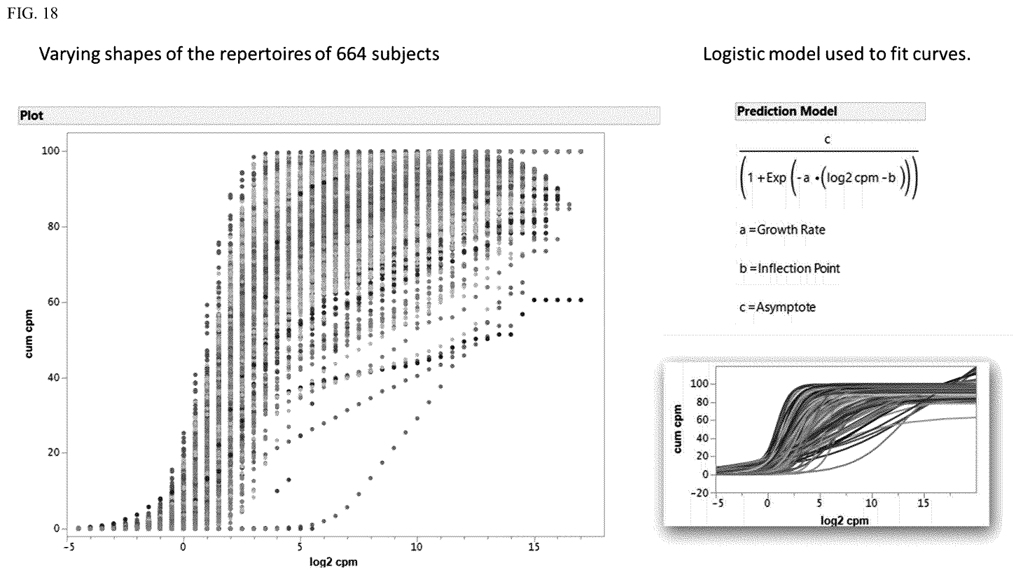

A different way of visualizing the differences is by plotting the cumulative distribution patterns of the binned data. In addition, mathematical models can be used to quantify the clonal frequency patterns within an individual and to compare and contrast different individuals. The curves have a general sigmoid shape and so a sigmoid logistic curve can be used to fit the data. The coefficients change depending on age, disease state. They are also expected to change over time during a therapeutic treatment. An example is shown in FIG. 18

When individuals are classified by age it becomes apparent that there is a characteristic T cell repertoire profile associated with the progression of age (FIG. 28). This is a useful reference when assessing whether the diversity of repertoires of individuals of diverse ages corresponds to what is normal for their age cohort.

### Example 7. Comparative Repertoires in CMV Infection

In some cases examination of the repertoires indicate an unevenness in distribution that has clinical significance. This is the case for individuals that have a positive serological status for the CMV herpes virus. Individuals with a CMV+ status tend to have a deficit in intermediate frequency clonotypes with a predominant subset of clonotypes that are over-represented in the repertoire. This is illustrated in FIGS. 19-20 across 664 T cell repertoires of CMV seropositive and seronegative subjects.

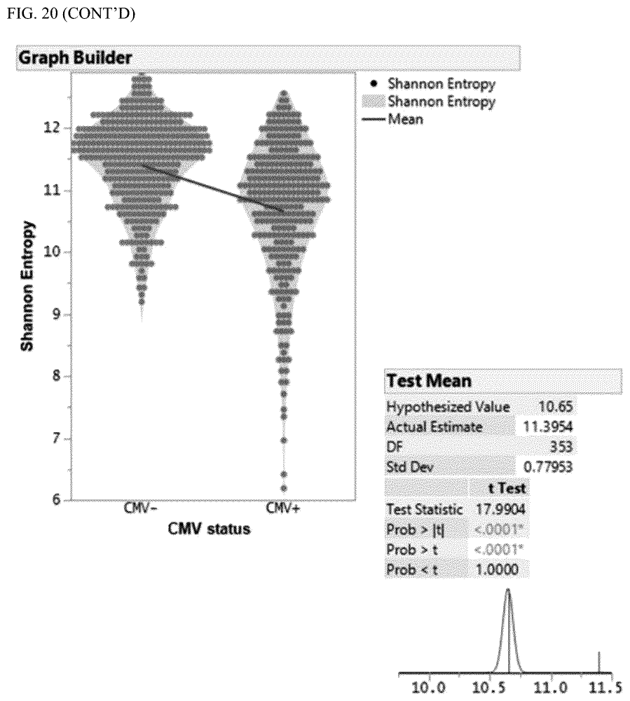

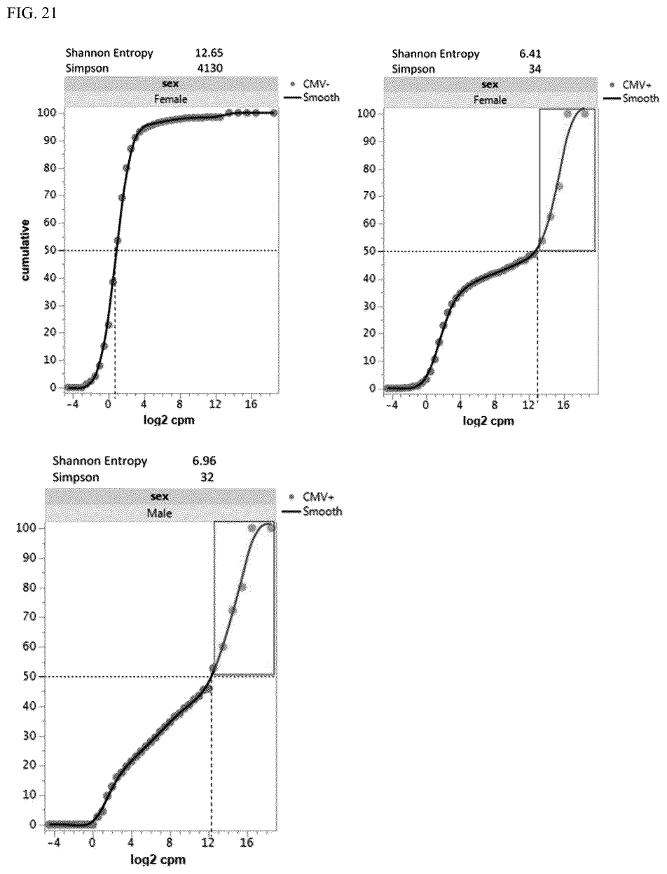

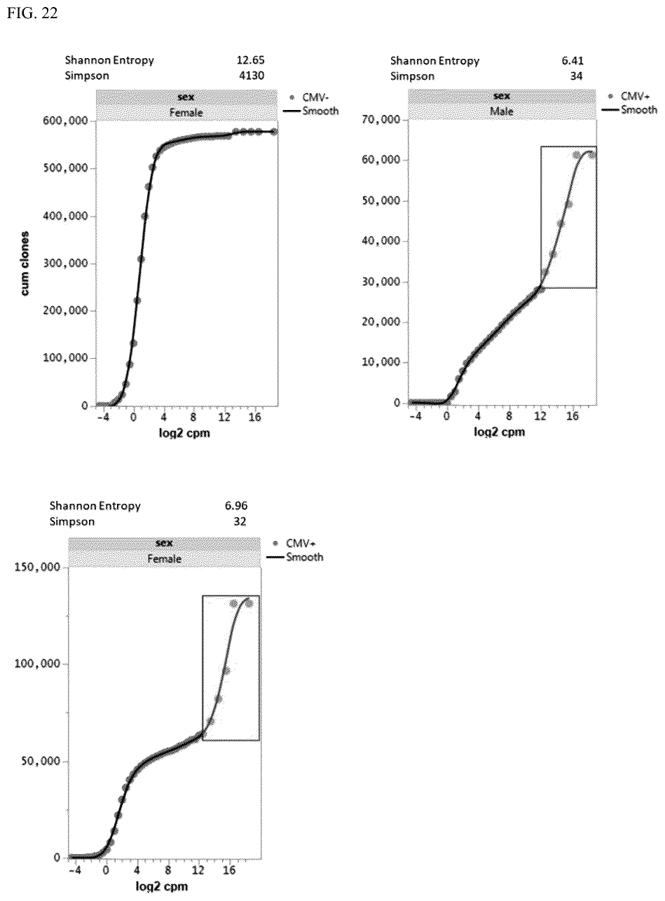

The cumulative distribution pattern of T cell beta variant (TCBV) clonotypes of 3 subjects with total clonotypes standardized to 100% were compared. All subjects in the A*02 MHC group. FIG. 21 shows that 50% of the entire repertoire is in the highly expanded subset of clonotypes. As there is a fixed total pool size there is a substantial loss of diversity as a result. The Shannon entropy and Simpson diversity index that are different measures of repertoire diversity are shown. In FIG. 22 the difference in the actual number of clonotypes is shown. The highly expanded subset in the highlighted area totals 30-60,000 clonotypes is noted. The highly expanded clones are likely the subset that are responding to the chronic CMV infection.

### Example 8: Repertoires in Autologous Transplants

PBMC (peripheral blood mononucleocytes) were collected from 4 patients who had undergone hematopoietic cell abrogation and autologous transplants. The cells were sorted to capture CD4 and CD8 T cells for TCR sequencing and B cells were isolated for sequence determination of IgA, IgD, IgG, and IgM isotypes.

Results of the sequencing generates a table of sequences from with the clonal frequency and the number of copies of each particular clone. The frequencies are normalized to the total number of sequences accumulated to account for differences between individuals due to differences in cell count or differences in efficiency of the sorting process. As the frequencies have many leading zeros they are typically transformed by multiplication by 10⁶ to give a metric equivalent of cells/million (CPM) that represents a number typically considered in laboratory work with cells. A base 2 logarithm is then computed from the CPM value and used for the binning process.

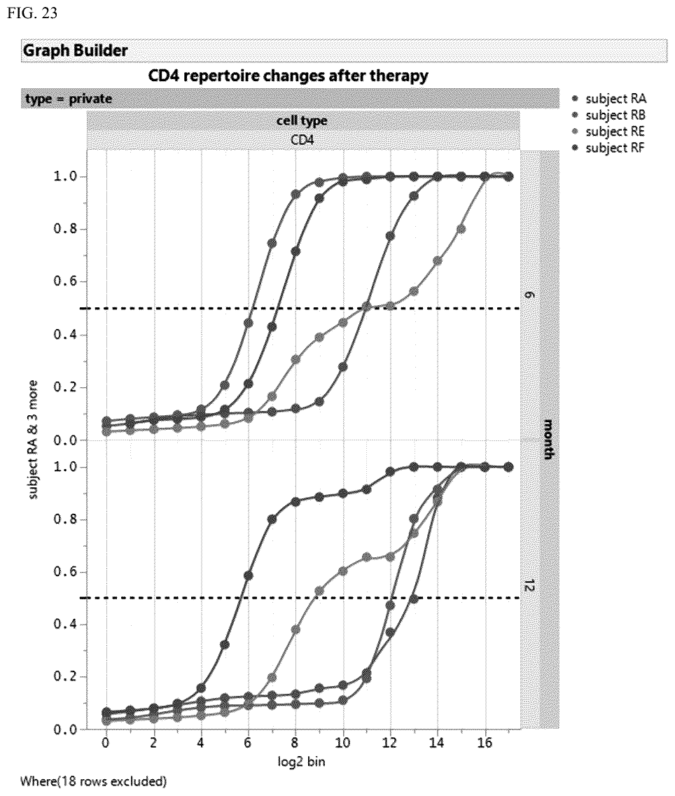

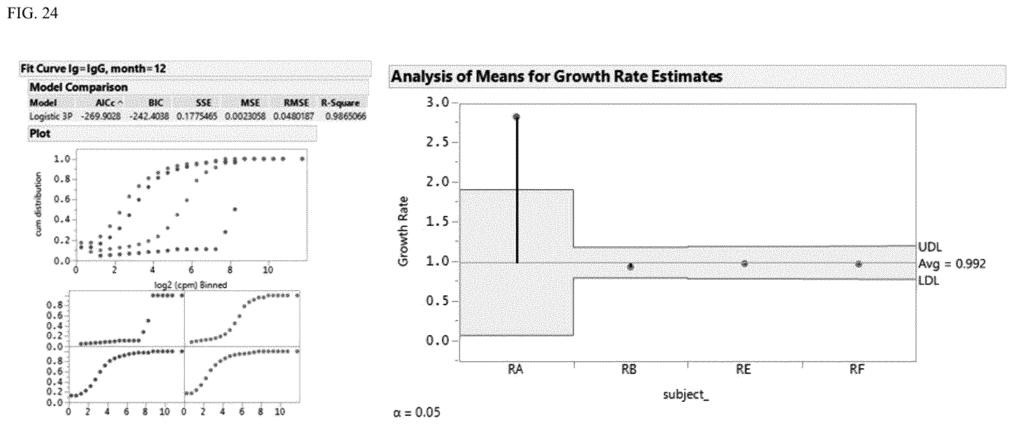

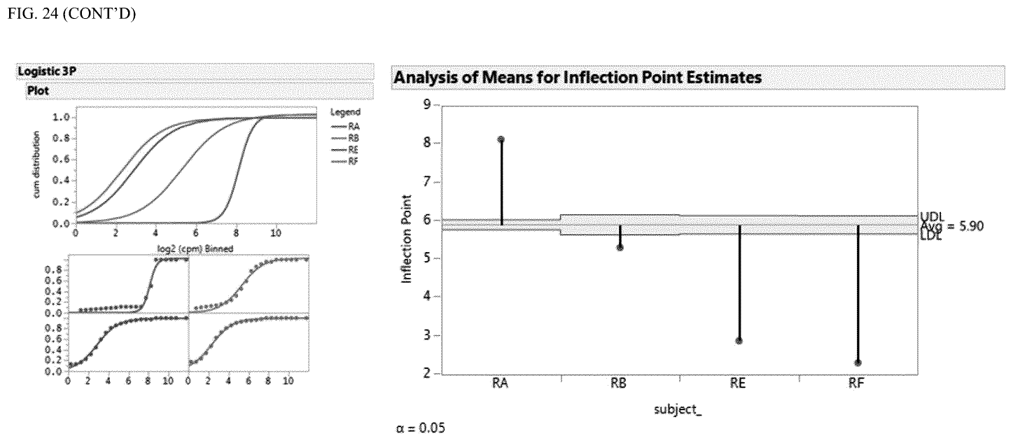

The observations are shown in FIGS. 23 and 24. The most notable change is for subject RB. In this subject at 6 months after treatment initiation half of the cells in the repertoire had a clonal frequency less than 2⁶. (CF50=clonal frequency 50%). In fact, this individual has the most diverse repertoire with subject RF being slightly less diverse at CF50=27. The repertoire of subject RE shows two obvious sub-distributions one with an FC50 of 7 and a second at approximately 14. After 12 months of treatment subject RB had developed a repertoire with a small number of very dominant clones, whereas the repertoire of subject RF had shifted towards a greater diversity with a FC50 of approximately 5.5. Subject RF was in disease remission and subject RB died.

Alternatively, logistic regression algorithms can be used to carry out statistical analysis of the datasets. Logistic regression generates a sigmoid curve that is characterized by an inflection point in the curve as well as a “growth rate” parameter that is a measure of the slope of the sigmoid.

### Example 9: Personalized Medicine Application of TCEM Motif Frequencies in Tumors

This example shows the application of frequency pattern analysis to the mutations identified in proteins in a biopsy from a single glioblastoma patient. Based on biopsies of the tumor and normal tissue, mutations were identified in ten proteins of interest. We examined the T cell exposed motifs which would be exposed to CD8 cytotoxic lymphocytes following MHC 1 presentation of peptides where the mutated amino acid was located in the T cell exposed motif. As the TCEM encompasses 5 contiguous amino acids, five TCEM were evaluated for each mutated protein. Analysis of TCEM frequency and the frequency of these motifs in the human proteome is shown in Table 3.

TABLE 3

| | | | | | SEQ | | SEQ | | SEQ |
|---|---|---|---|---|---|---|---|---|---|
| | | | | | ID | | ID | TCEM I | ID |
| Protein | gi wt | Protein | pos | peptide mut | NO: | peptide wt | NO: | mut | NO: |
| 1 | 22027642 | kelch-like | 607 | AVTMEPCWK | 1 | AVTMEPCRK | 51 | MEPCW | 101 |
| | | ECH-associated | 608 | VTMEPCWKQ | 2 | VTMEPCRKQ | 52 | EPCWK | 102 |
| | | protein 1 | 609 | TMEPCWKQI | 3 | TMEPCRKQI | 53 | PCWKQ | 103 |
| | | | 610 | MEPCWKQID | 4 | MEPCRKQID | 54 | CWKQI | 104 |
| | | | 611 | EPCWKQIDQ | 5 | EPCRKQIDQ | 55 | WKQID | 105 |
| 2 | 18765694 | dipeptidyl | 49 | LKNTYRLML | 6 | LKNTYRLKL | 56 | TYRLM | 106 |
| | | peptidase 4 | 50 | KNTYRLMLY | 7 | KNTYRLKLY | 57 | YRLML | 107 |
| | | | 51 | NTYRLMLYS | 8 | NTYRLKLYS | 58 | RLMLY | 108 |
| | | | 52 | TYRLMLYSL | 9 | TYRLKLYSL | 59 | LMLYS | 109 |
| | | | 53 | YRLMLYSLR | 10 | YRLKLYSLR | 60 | MLYSL | 110 |
| 3 | 30089972 | peroxisomal | 119 | QQERFFMLA | 11 | QQERFFMPA | 61 | RFFML | 111 |
| | | acyl-coenzyme | 120 | QERFFMLAW | 12 | QERFFMPAW | 62 | FFMLA | 112 |
| | | A oxidase 1 | 121 | ERFFMLAWN | 13 | ERFFMPAWN | 63 | FMLAW | 113 |
| | | isoform a | 122 | RFFMLAWNL | 14 | RFFMPAWNL | 64 | MLAWN | 114 |
| | | | 123 | FFMLAWNLE | 15 | FFMPAWNLE | 65 | LAWNL | 115 |
| 4 | 166064029 | | 408 | SAMPRAQLS | 16 | SAMPRAQPS | 66 | PRAQL | 116 |
| | | | 409 | AMPRAQLSS | 17 | AMPRAQPSS | 67 | RAQLS | 117 |
| | | | 410 | MPRAQLSSA | 18 | MPRAQPSSA | 68 | AQLSS | 118 |
| | | | 411 | PRAQLSSAS | 19 | PRAQPSSAS | 69 | QLSSA | 119 |
| | | | 412 | RAQLSSASY | 20 | RAQPSSASY | 70 | LSSAS | 120 |
| 5 | 41281911 | coiled-coil | 115 | LLQEKELPE | 21 | LLQEKELQE | 71 | EKELP | 121 |
| | | domain-containing | 116 | LQEKELPEE | 22 | LQEKELQEE | 72 | KELPE | 122 |
| | | protein 50 | 117 | QEKELPEEK | 23 | QEKELQEEK | 73 | ELPEE | 123 |
| | | long isoform | 118 | EKELPEEKK | 24 | EKELQEEKK | 74 | LPEEK | 124 |
| | | | 119 | KELPEEKKR | 25 | KELQEEKKR | 75 | PEEKK | 125 |
| 6 | 4758650 | kinesin | 485 | KEVLQALKE | 26 | KEVLQALEE | 76 | LQALK | 126 |
| | | heavy chain | 486 | EVLQALKEL | 27 | EVLQALEEL | 77 | QALKE | 127 |
| | | isoform 5C | 487 | VLQALKELA | 28 | VLQALEELA | 78 | ALKEL | 128 |
| | | | 488 | LQALKELAV | 29 | LQALEELAV | 79 | LKELA | 129 |
| | | | 489 | QALKELAVN | 30 | QALEELAVN | 80 | KELAV | 130 |
| 7 | 124028529 | symplekin | 1062 | GAVFDKCSE | 31 | GAVFDKCPE | 81 | FDKCS | 131 |
| | | | 1063 | AVFDKCSEL | 32 | AVFDKCPEL | 82 | DKCSE | 132 |
| | | | 1064 | VFDKCSELR | 33 | VFDKCPELR | 83 | KCSEL | 133 |
| | | | 1065 | FDKCSELRE | 34 | FDKCPELRE | 84 | CSELR | 134 |
| | | | 1066 | DKCSELREP | 35 | DKCPELREP | 85 | SELRE | 135 |
| 8 | 301171467 | ATP-dependent | 474 | DRSQRDRKE | 36 | DRSQRDREE | 86 | QRDRK | 136 |
| | | RNA helicase | 475 | RSQRDRKEA | 37 | RSQRDREEA | 87 | RDRKE | 137 |
| | | DDX3X | 476 | SQRDRKEAL | 38 | SQRDREEAL | 88 | DRKEA | 138 |
| | | isoform 2 | 477 | QRDRKEALH | 39 | QRDREEALH | 89 | RKEAL | 139 |
| | | | 478 | RDRKEALHQ | 40 | RDREEALHQ | 90 | KEALH | 140 |
| 9 | 73765544 | phosphatidy | 14 | XXMTAIIEE | 41 | XXMTAIIKE | 91 | TAIIE | 141 |
| | | linositol 3 | 15 | XMTAIIEEI | 42 | XMTAIIKEI | 92 | AIIEE | 142 |
| | | | 16 | MTAIIEEIV | 43 | MTAIIKEIV | 93 | IIEEI | 143 |
| | | | 17 | TAIIEEIVS | 44 | TAIIKEIVS | 94 | IEEIV | 144 |
| | | | 18 | AIIEEIVSR | 45 | AIIKEIVSR | 95 | EEIVS | 145 |
| 10 | 23510323 | nephrocystin-4 | 36 | ARQPWKEPT | 46 | ARQPWKEST | 96 | PWKEP | 146 |
| | | isoform a | 37 | RQPWKEPTA | 47 | RQPWKESTA | 97 | WKEPT | 147 |
| | | | 38 | QPWKEPTAF | 48 | QPWKESTAF | 98 | KEPTA | 148 |
| | | | 39 | PWKEPTAFQ | 49 | PWKESTAFQ | 99 | EPTAF | 149 |
| | | | 40 | WKEPTAFQC | 50 | WKESTAFQC | 100 | PTAFQ | 150 |

| | | | | | Human | Human | | |
|---|---|---|---|---|---|---|---|---|
| | | SEQ | | | proteome | proteome | | delta |
| | TCEM I | ID | TCEM I | TCEM I | frequency | frequency | delta | Human |
| Protein | wt | NO: | Fc mut | Fc wt | mut | wt | Fc | Frequency |
| 1 | MEPCR | 151 | 23 | 23 | −3.66 | −2.45 | 0 | 1.21 |
| | EPCRK | 152 | 24 | 24 | −0.96 | −1.21 | 0 | −0.24 |
| | PCRKQ | 153 | 24 | 24 | −3.66 | −2.04 | 0 | 1.62 |
| | CRKQI | 154 | 24 | 23 | −2.04 | −3.66 | 1 | −1.62 |
| | RKQID | 155 | 24 | 21 | −3.66 | 0.04 | 3 | 3.71 |
| 2 | TYRLK | 156 | 23 | 22 | −3.66 | −1.36 | 1 | 2.30 |
| | YRLKL | 157 | 23 | 22 | −0.61 | 0.11 | 1 | 0.72 |
| | RLKLY | 158 | 22 | 19 | −1.21 | −0.25 | 3 | 0.96 |
| | LKLYS | 159 | 18 | 13 | −2.45 | 0.97 | 5 | 3.42 |
| | KLYSL | 160 | 20 | 19 | −1.54 | 1.09 | 1 | 2.63 |
| 3 | RFFMP | 161 | 22 | 22 | −2.04 | −2.04 | 0 | 0.00 |
| | FFMPA | 162 | 24 | 23 | −3.16 | −1.54 | 1 | 1.63 |
| | FMPAW | 163 | 24 | 24 | −0.86 | −0.61 | 0 | 0.25 |
| | MPAWN | 164 | 23 | 22 | −3.66 | −2.45 | 1 | 1.21 |
| | PAWNL | 165 | 23 | 20 | −1.08 | −2.45 | 3 | −1.37 |
| 4 | PRAQP | 166 | 21 | 21 | 0.35 | 0.57 | 0 | 0.23 |
| | RAQPS | 167 | 17 | 18 | 0.15 | 0.29 | −1 | 0.15 |
| | AQPSS | 168 | 19 | 19 | 1.47 | 1.41 | 0 | −0.06 |
| | QPSSA | 169 | 18 | 11 | 1.43 | 0.70 | 7 | −0.73 |
| | PSSAS | 170 | 13 | 10 | 2.11 | 2.86 | 3 | 0.75 |
| 5 | EKELQ | 171 | 22 | 22 | 0.65 | 1.55 | 0 | 0.90 |
| | KELQE | 172 | 21 | 21 | 1.28 | 1.72 | 0 | 0.44 |
| | ELQEE | 173 | 21 | 21 | 1.17 | 1.46 | 0 | 0.29 |
| | LQEEK | 174 | 22 | 22 | 1.02 | 1.58 | 0 | 0.56 |
| | QEEKK | 175 | 23 | 23 | 1.53 | 1.10 | 0 | −0.43 |
| 6 | LQALE | 176 | 22 | 22 | 1.19 | 1.89 | 0 | 0.70 |
| | QALEE | 177 | 21 | 23 | 1.61 | 1.81 | −2 | 0.19 |
| | ALEEL | 178 | 14 | 20 | 1.68 | 2.21 | −6 | 0.53 |
| | LEELA | 179 | 21 | 20 | 1.13 | 1.63 | 1 | 0.50 |
| | EELAV | 180 | 16 | 18 | 0.15 | 1.02 | −2 | 0.87 |
| 7 | FDKCP | 181 | 24 | 24 | −3.16 | −2.45 | 0 | 0.71 |
| | DKCPE | 182 | 23 | 23 | −1.21 | −1.08 | 0 | 0.13 |
| | KCPEL | 183 | 16 | 22 | 0.08 | −0.36 | −6 | −0.44 |
| | CPELR | 184 | 16 | 22 | −0.25 | −0.20 | −6 | 0.05 |
| | PELRE | 185 | 20 | 22 | 1.22 | 1.26 | −2 | 0.05 |
| 8 | QRDRE | 186 | 22 | 22 | −1.36 | 0.24 | 0 | 1.60 |
| | RDREE | 187 | 16 | 16 | −0.16 | 0.35 | 0 | 0.50 |
| | DREEA | 188 | 22 | 18 | −0.20 | 0.45 | 4 | 0.65 |
| | REEAL | 189 | 21 | 18 | 1.12 | 0.90 | 3 | −0.22 |
| | EEALH | 190 | 20 | 22 | −0.61 | 0.80 | 2 | 1.41 |
| 9 | TAIIK | 191 | 20 | 19 | −0.20 | −0.03 | 1 | 0.17 |
| | AIIKE | 192 | 21 | 22 | 0.86 | 0.29 | −1 | −0.57 |
| | IIKEI | 193 | 22 | 21 | 0.32 | 0.42 | 1 | 0.10 |
| | IKEIV | 194 | 23 | 20 | −0.47 | 0.21 | 3 | 0.68 |
| | KEIVS | 195 | 21 | 19 | 0.47 | 0.32 | 2 | −0.15 |
| 10 | PWKES | 196 | 23 | 23 | −2.45 | −0.61 | 0 | 1.84 |
| | WKEST | 197 | 23 | 23 | −3.16 | −1.36 | 0 | 1.80 |
| | KESTA | 198 | 19 | 18 | −0.69 | −0.11 | 1 | 0.58 |
| | ESTAF | 199 | 22 | 20 | −0.77 | 0.65 | 2 | 1.42 |
| | STAFQ | 200 | 22 | 21 | 0.29 | 0.29 | 1 | 0.00 |

Table 1 shows the Frequency Category in the human immunoglobulinome as a log₂ of the occurrence in the reference data base of ˜40 million immunoglobulin variable regions; hence Fc20 represents 1 in 2²⁰ or 1 in 1,048,576 and Fc24 is 1 in >8.3 million. The Frequency of occurrence pf TCEM in the Human proteome is based on the entire human proteome including all isoforms (approximately 88,000 proteins) and is shown in standard deviations units above or below the mean of zero. Delta columns show the difference between wild type and mutated TCEM values and the frequency in the human proteome, where positive values are indicative of an increase in rarity of the motifs in the mutated proteins.

Table 3 shows that the mutated peptides have TCEM 1 which are more rare in the human proteome and in most cases are more rare in the human immunoglobulinome. Several of the mutated peptides have TCEM 1 which are more than 3 standard deviation units below the mean frequency of occurrence in the human proteome.

We then identified which proteins in the human proteomes carried any one of the 50 unique TCEM identified in the 10 mutant proteins; overall 503 were identified as carrying these pentameric motifs and these proteins were evaluated further. The relative MHC allele binding of the peptides which carried those TCEM was computed for those alleles carried by this patient. Among the 503, 213 proteins (including fragments of some proteins reflected in additional Uniprot entries) were identified in which there was a matching TCEM as well as a predicted binding to one or more of the patient's MHC 1 alleles in excess of 1 standard deviation below the mean (I.e. at least moderate binding). These were peptides with potential for off target interactions if used as neoepitope vaccines. Among these proteins we evaluated the potential significance of off target responses. Notably the two mutated proteins from which peptide vaccines had elicited the strongest ELISPOT results in this patient were those where no matches were found in the proteome, suggesting that the most rare peptide motifs elicited the greatest de novo responses.

Together these analyses indicate how comparing TCEM motifs from a tumor biopsy to the frequency patterns in reference human proteome and immunoglobulinome may assist in design of immunotherapeutic interventions.

### Example 10: Repertoires in Pathogens: Prediction of Influenza Virulence

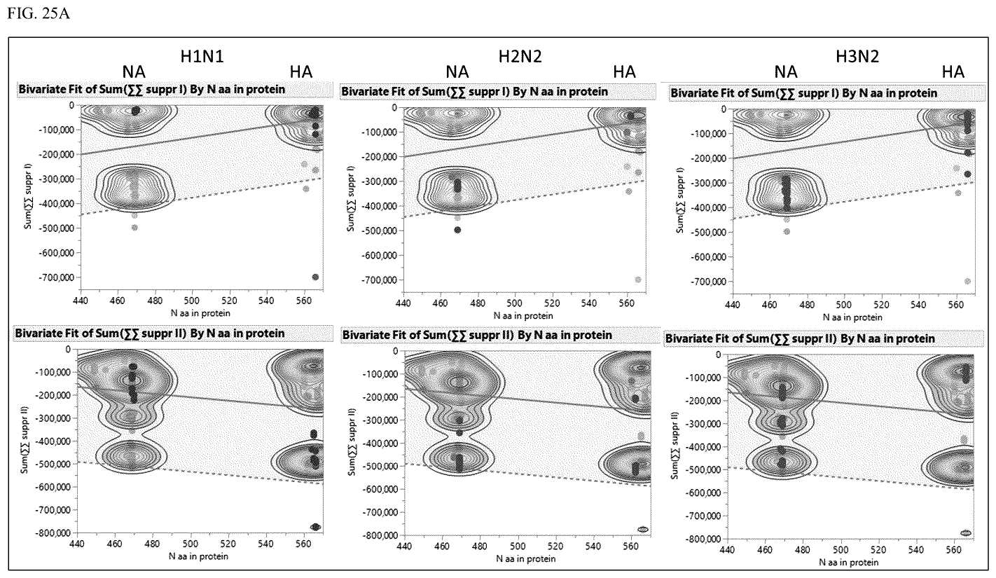

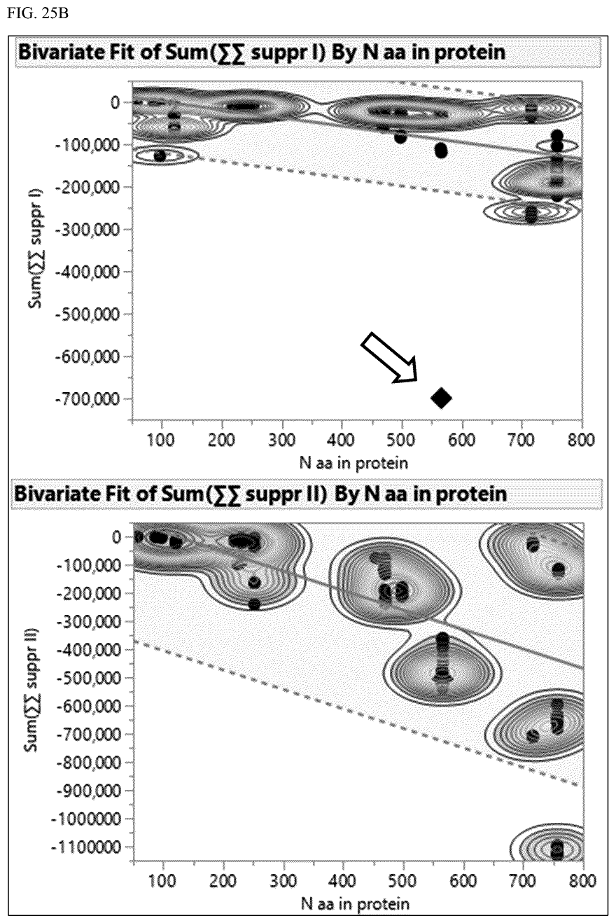

Using as a reference the frequency distribution of T cell exposed motifs in the overall immunoglobulinome [4] (based on approximately 40 million IgV sequences analyzed), we categorized the frequency of each TCEM in a random sample of influenza A hemagglutinins representing each HA type. Two conserved features, the HA1 receptor binding site and the HA2 stalk epitope are flanked by more common TCEM less likely to result in a strong Th response and memory; the stalk epitope also lacks peptides with strong predicted MHC binding. We derived an index of suppressive or stimulation potential, based on TCEM frequency multiplied by HLA predicted to bind above threshold as an indicator of the probability of a T regulatory response within a human population (with obviously individual differences by allele) and compared the suppressive and stimulation index of HA and NA across a stratified random subset of H1N1, H2N2, H3N2, and other HA types isolated from humans. FIG. 25A shows results for H1N1, H2N2, and H3N2, and that each type has a characteristic “stimulation vs suppressive signature”. Notably among NA, N2 appear more suppressive than other NA, and HA H1 more so than H2 and H3. When we compared (FIG. 25B) the suppressive signature of all proteins in a set of 66 H1N1 across the last 100 years, we noted that A/Brevig Mission/l/1918 is a clear outlier, containing in its HA an extremely common MHC I TCEM motif that is present in 50% of all Ig variable regions. Transcriptional frequency will affect the impact of each protein and so while other proteins, particularly PA, have a higher MHC II suppressive index, they are present in smaller numbers than the NP, M1, HA and NA. The motif in HA of Brevig is remarkable (and is also found in other 1918 isolates). The HA of 1918 has been shown to be essential to its virulence. Such a motif might be expected to elicit a T regulatory response suppressing the CD8+ cytotoxic function, allowing a more severe viral pneumonia and extended shedding and transmission. We do not suggest that this could be a single marker of virulence, but it may signal a contributing factor (with other viral, societal and secondary infection factors) which merits further examination and may flag pandemic potential.

This provides an example of the application of frequency patterns of TCEM to gain understanding of the immunopathogenesis of a pathogen and to guide development of immunotherapeutic and prophylactic interventions.

### Example 11: TCEM Patterns of Diversity Following T Cell Ablation and Stem Cell Transplant

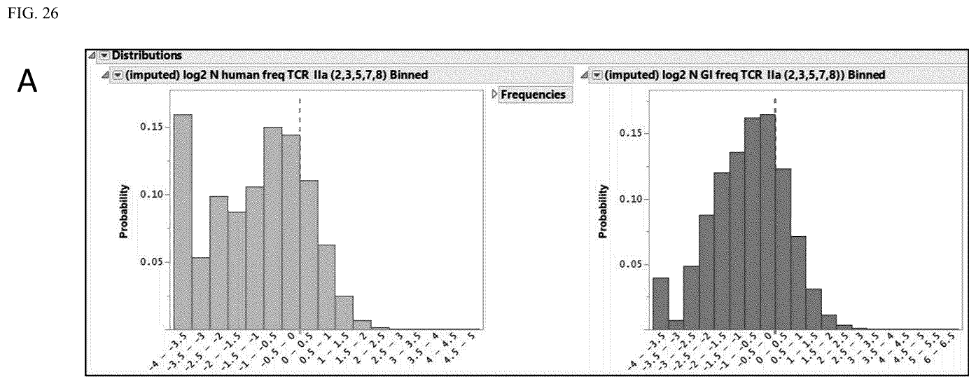

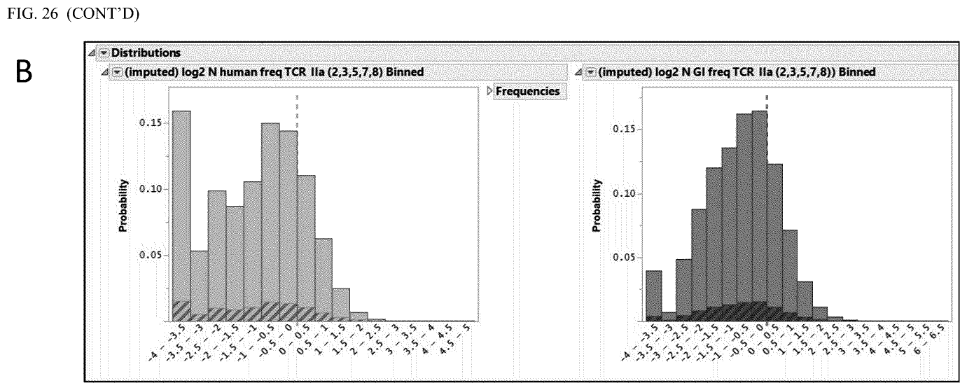

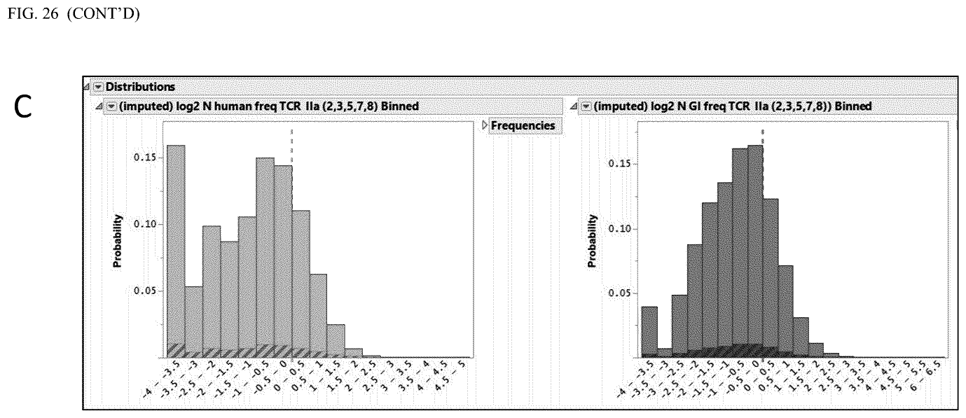

A group of sixteen patients suffering from a variety of hematologic cancers were subjected to chemotherapeutic B cell ablation followed by transplant of bone marrow stem cells from HLA matched donors. B cells were extracted from PBMC samples prior to ablation and at 3, 6 and 12 months following transplant. CDR and VDJ regions of the BCR were sequenced. We extracted TCEM motifs from these sequences and arrayed them by clonoptype frequency for each patient and for the aggregate group of patients. Distributions of TCEM motifs were then compared among the group and with reference TCEM distributions found in the normal human proteome, immunoglobulinome and gastrointestinal microbiome. FIG. 26A shows the patterns of TCEM IIa in the BCR of all patients in the dataset compared to human proteome and gastrointestinal microbiome normal distribution. The frequency distributions in the reference proteomes of the human and the GI microbiome organisms have been normalized to zero mean unit variance log normal distributions indicated by the dashed lines and are binned by half-standard deviation unit bins. The left-most bin in each histogram represents motifs that are absent from that distribution. Several features can be noted: 1) the human proteome and GI microbiome have different distribution properties, 2) the distribution of TCEM IIa generated by immunoglobulin somatic mutation in this patient group is skewed toward slightly more rare motifs in both of the reference proteomes, and 3) the immunoglobulin somatic mutations generates broad matches to both reference distributions. FIGS. 26B and 26C show the TCEM repertoires of patients 1 and 10 relative to the group as a whole and show that patient 1 has generated more motifs matching those in proteome and gastrointestinal microbiome than patient 10.

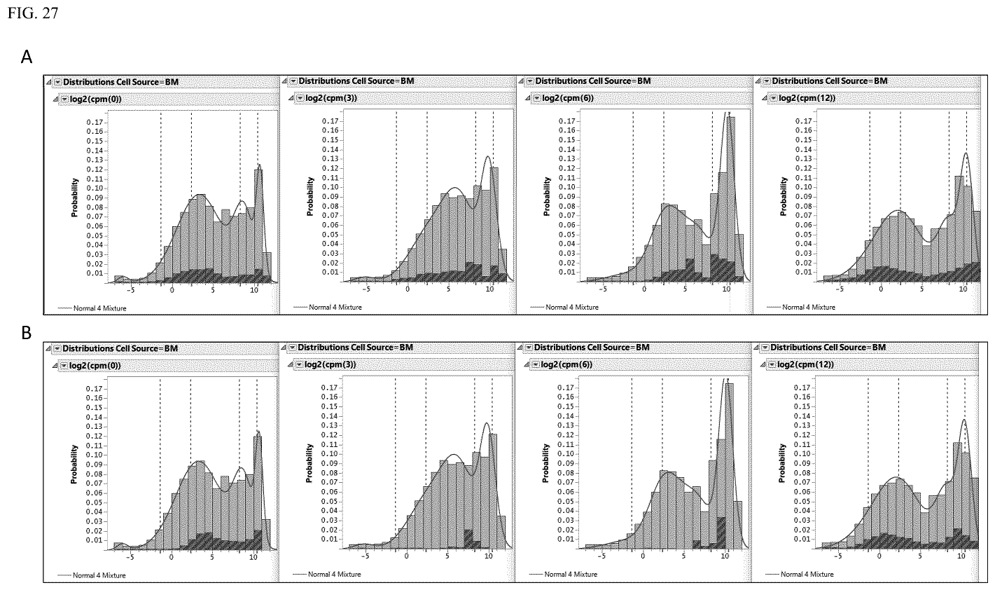

FIG. 27 tracks the patients over time, showing the pattern of TCEM IIa distribution before diseased repertoire ablation (time 0) and at 3, 6, and 12 months after bone marrow transplant of HLA matched donors. Frequency of TCEM IIa in the different subjects was standardized by multiplying the frequency of each by 10⁶ and placed in log 2 frequency bins (x-axis). The y-axis is the relative proportion of the total distribution found in any of the individual bins. The distributions are modeled as a 4-normal distribution mixture (red line). The dashed lines at generated from the 12 monthdata model and are centered on the underlying modeled distribution means. These points are used as reference frequencies in the other distributions and show the expansion of more rare motifs over time. Patient 1 shows a relatively consistent repertoire expansion over time (FIG. 27A), whereas Patient 10 (FIG. 27B) has a relatively poor expansion at the 3 and 6 month time points, but is improving at 12 months, although not equivalent to Patient 1.

### Example 11. Binning Identifies Diagnostic Clonality Patterns of Immunoglobulin Proteins

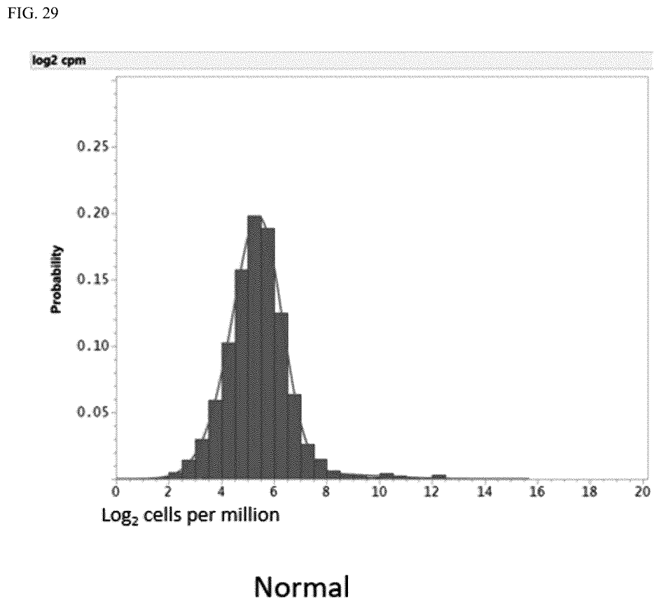

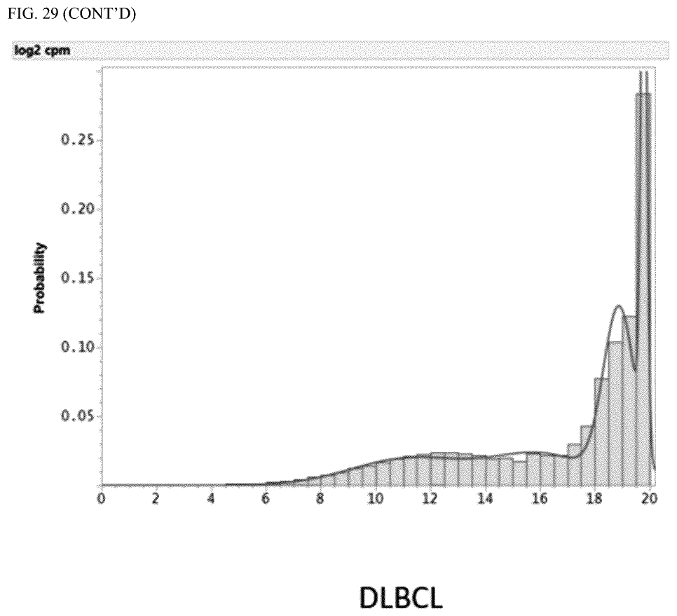

When binning of repertoire sequences is applied as described in Example 6 to the immunoglobulin sequences of patients affected by leukemia, characteristic patterns are noted which differ markedly from the distributions in normal individuals. A set of 39.73 million immunoglobulin FW3 and CDR3 nucleotide sequences from a population of healthy individuals was assembled. Nucleotide sequences were translated to amino acid sequences and the clonal diversity determined as described in Example 6. A distinctive pattern of clonal diversity is noted for the leukemic patients as compared with normal patients as shown in FIG. 29.

### Example 12: Many Nucleotide—One Protein

Based on the immunoglobulin variable regions sequences from a normal population and for a group of leukemic patients, the relationship of nucleotide sequence diversity and protein sequence diversity was examined. The relative amino acid sequence diversity was evaluated both for the CDR3 region and on the variable region as a whole.

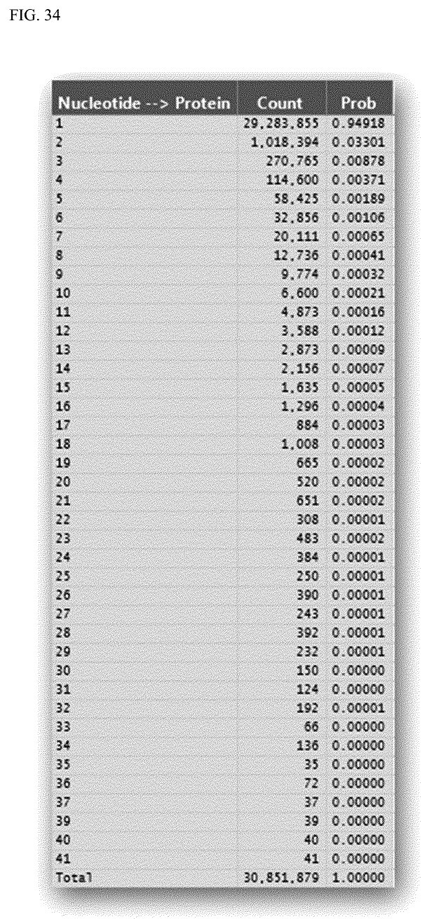

In the normal set of 39.73 million immunoglobulin sequences the occurrence of many nucleotide to one protein sequences was relatively low, with 95% of all protein sequences having a single unique coding sequence. Of the remaining 1,018,394 sequences are encoded by 2 nucleotide sequences, and the remaining 549,640 protein sequences (<5%) are encoded by 3-40 different nucleotide sequences each (FIG. 34). The net result is that the 39.73 million nucleotide sequences resulted in 30.85 million protein sequences.

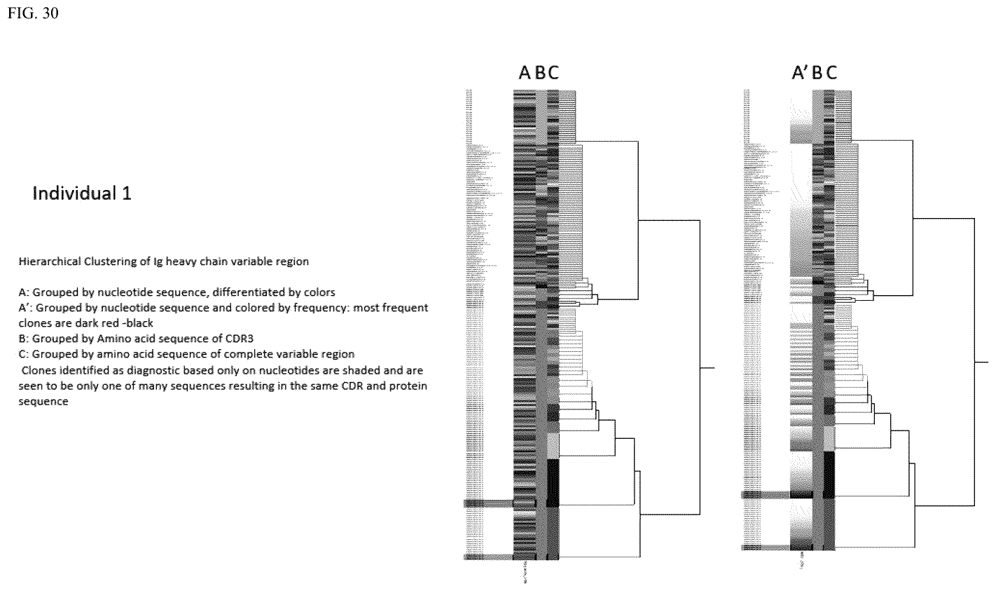

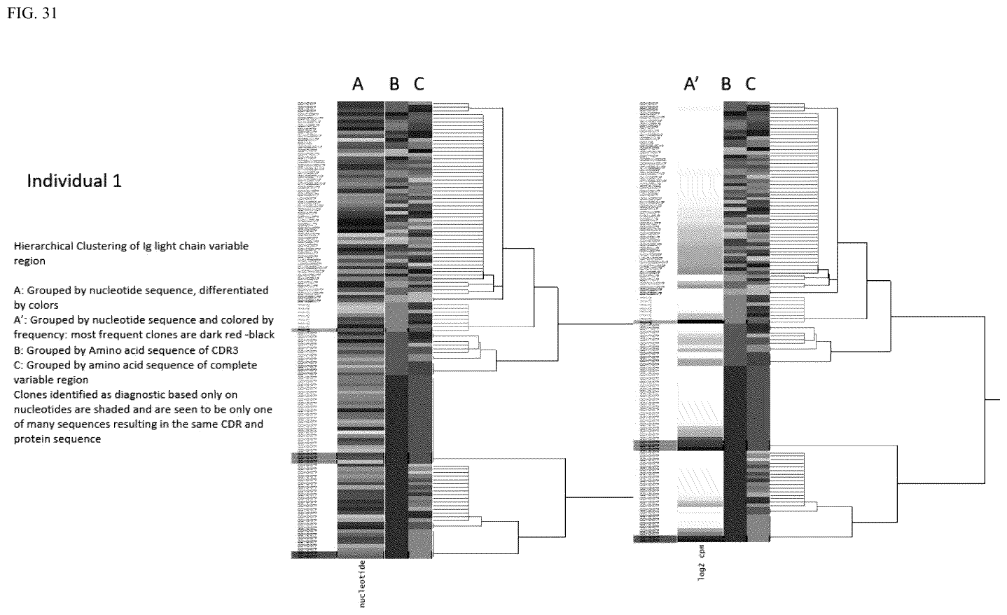

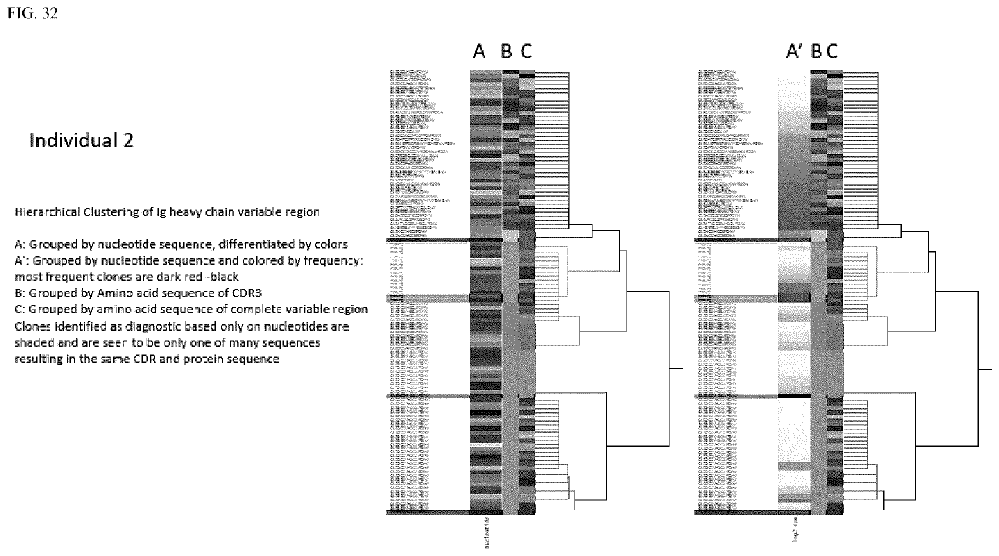

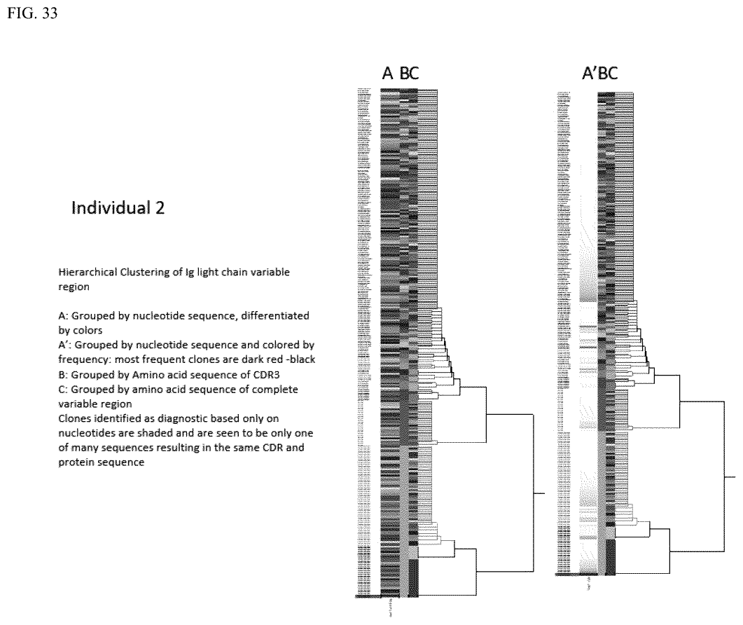

In a set of 380 patients affected by diffuse large B-cell lymphoma (DLBCL) the number of proteins encoded by many different nucleotide sequences were much higher. For some particular patients an overall ratio of 10 synonymous nucleotide sequences to one CDR3 protein sequence was noted in the pathologic sequences. The correspondence of nucleotide sequence numbers to protein numbers are shown for two such patients for both heavy and light immunoglobulin chains in FIGS. 30-33. In each it is seen that multiple nucleotide sequences all encode for one CDR and this is found in several Ig variable regions. For Individual 1 the largest heavy chain CDR amino acid sequence is encoded by 27 different nucleotide sequences. For Individual 2 the largest heavy chain CDR amino acid sequence is encoded by 25 different nucleotide sequences. A similar but numerically different pattern of many to one relationships exists in both heavy and in light chain sequences.

B cells process their [7] endogenous immunoglobulins into peptides and present peptides on MHC which stimulate corresponding T cell help leading to clonal expansion [8]. When multiple clonal lines of B cells share the same protein sequence, albeit from different nucleotide origins, they would also share the same T cell help and expand in parallel. In the absence of an apototic signal or other suppressive signal to curtail such T cell help, as is the case in B cells carrying a tumor gene mutation such as p53 or CCND1, this may result in an unrestrained B cell expansion that extends to all clonal lines that engage the same cognate T cell help. Such many to one relationships of nucleotide sequences to protein sequences may be indicative of daughter clonal lines or may represent selection of bystander clones based on their B-T cell interaction and stimulation therefrom. The degree to which a multiplicity of immunoglobulin nucleotide sequences is transcribed to the same protein is excessive in DBLCL indicates it is an additional diagnostic indicator for this and potentially other leukemias. It is therefore important to make determinations on interventions based on the protein sequence, which determines T cell interaction, and not only on the nucleotide sequence which may fail to target many B cells with the same or similar functionality and/or pathology. Targeting based only on nucleotide sequence may significantly underestimate the size of the clones dominating and driving the leukemia or other B cell disease.

### Example 13: Analysis of TCEM Frequencies in Allergens

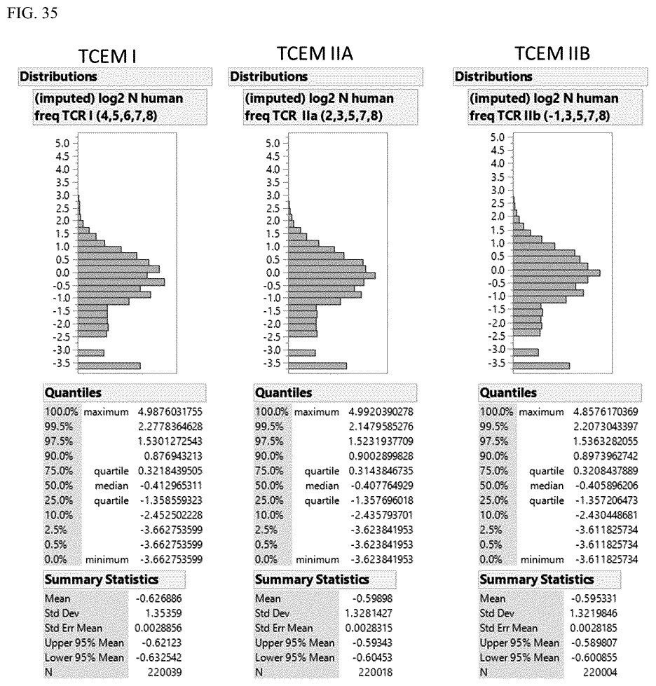

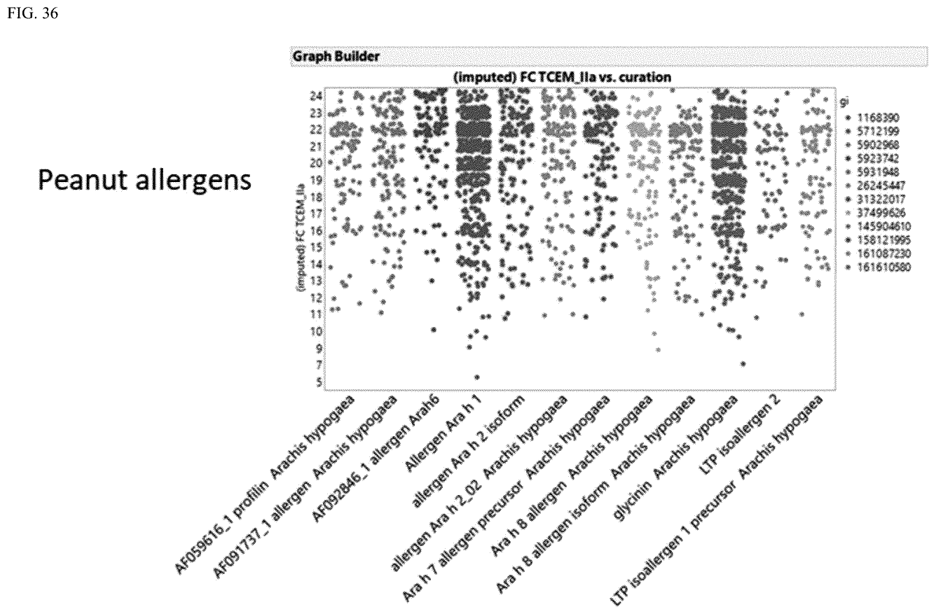

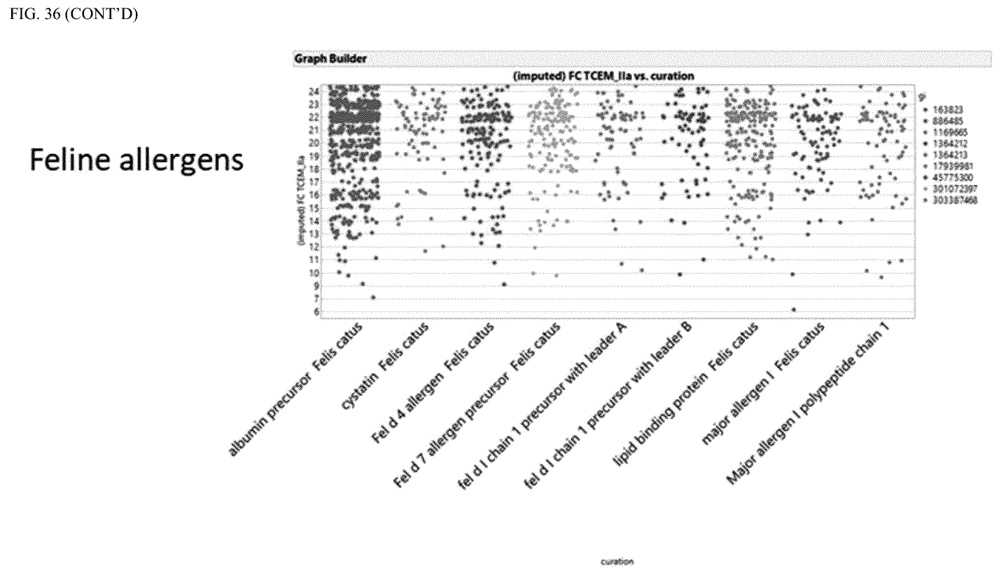

The sequences of over 1000 allergen proteins were assembled including proteins from animal, plant, fungal, insect, mite, salivary, and helminth sources which are known or suspected of causing allergies by aerosol exposure, ingestion or skin contact. Sequences below 50 amino acids and duplicate sequences were excluded, leaving 848 unique sequences. TCEM motifs extracted from these proteins were compared to the frequency distributions in the human proteome and immunoglobulin and found to differ markedly in their distribution. Allergens comprised a significantly higher content of motifs that are very rare in the human proteome (FIG. 35), including many exceeding 3 standard deviations below the mean of the human proteome. When the frequency classification was compared with the human immunoglobulinome proteins differed individually but many comprised a large number of extremely rare motifs encountered in less than 1 in 8 million immunoglobulin variable regions. Two examples, for peanuts and allergens from cats are shown in FIG. 36.

### REFERENCE LIST

1. Lefranc M P, Giudicelli V, Ginestoux C, Jabado-Michaloud J, Folch G, Bellahcene F, et al. IMGT, the international ImMunoGeneTics information system. Nucleic acids research. 2009; 37 (Database issue):D1006-12. Epub 2008/11/04. doi: 10.1093/nar/gkn838. PubMed PMID: 18978023; PubMed Central PMCID: PMC2686541.
2. Birnbaum M E, Mendoza J L, Sethi D K, Dong S, Glanville J, Dobbins J, et al. Deconstructing the Peptide-MHC Specificity of T Cell Recognition. Cell. 2014; 157(5):1073-87. Epub 2014/05/27. doi: 10.1016/j.cell.2014.03.047. PubMed PMID: 24855945.
3. Rudolph M G, Stanfield R L, Wilson I A. How TCRs bind MHCs, peptides, and coreceptors. Annu Rev Immunol. 2006; 24:419-66. Epub 2006/03/23. doi: 10.1146/annurev.immunol.23.021704.115658. PubMed PMID: 16551255.
4. Bremel R D, Homan E J. Frequency Patterns of T-Cell Exposed Amino Acid Motifs in Immunoglobulin Heavy Chain Peptides Presented by MHCs. Frontiers in immunology. 2014; 5:541. doi: 10.3389/fimmu.2014.00541. PubMed PMID: 25389426; PubMed Central PMCID: PMC4211557.
5. Bremel R D, Homan J. Extensive T-cell epitope repertoire sharing among human proteome, gastrointestinal microbiome, and pathogenic bacteria: Implications for the definition of self. Frontiers in immunology. 2015; 6. doi: 10.3389/fimmu.2015.00538.
6. Li M O, Rudensky A Y. T cell receptor signalling in the control of regulatory T cell differentiation and function. Nature reviews Immunology. 2016; 16(4):220-33. doi: 10.1038/nri.2016.26. PubMed PMID: 27026074; PubMed Central PMCID: PMCPMC4968889.
7. Bogen B, Weiss S. Processing and presentation of idiotypes to MHC-Restricted T cells. International Reviews Immunology. 1993; 10:337-55.
8. Weiss S, Bogen B. B-lymphoma cells process and present their endogenous immunoglobulin to major histocompatibility complex-restricted T cells. Proc Natl Acad Sci USA. 1989; 86(1):282-6. Epub 1989/01/01. PubMed PMID: 2492101; PubMed Central PMCID: PMC286448.
9. Shreiner A B, Kao J Y, Young V B. The gut microbiome in health and in disease. Current opinion in gastroenterology. 2015; 31(1):69-75. doi: 10.1097/MOG.0000000000000139. PubMed PMID: 25394236; PubMed Central PMCID: PMCPMC4290017.
10. Belkaid Y, Hand T W. Role of the microbiota in immunity and inflammation. Cell. 2014; 157(1):121-41. doi: 10.1016/j.cell.2014.03.011. PubMed PMID: 24679531; PubMed Central PMCID: PMC4056765.
11. Belkaid Y, Rouse B T. Natural regulatory T cells in infectious disease. Nat Immunol. 2005; 6(4):353-60. doi: 10.1038/nil181. PubMed PMID: 15785761.
12. Cooper P J. Intestinal worms and human allergy. Parasite Immunol. 2004; 26(11-12):455-67. doi: 10.1111/j.0141-9838.2004.00728.x. PubMed PMID: 15771681.
13. Wammes L J, Mpairwe H, Elliott A M, Yazdanbakhsh M. Helminth therapy or elimination: epidemiological, immunological, and clinical considerations. The Lancet infectious diseases. 2014; 14(11):1150-62. doi: 10.1016/S1473-3099(14)70771-6. PubMed PMID: 24981042.
14. Gopalakrishnan V, Spencer C N, Nezi L, Reuben A, Andrews M C, Karpinets T V, et al. Gut microbiome modulates response to anti-PD-1 immunotherapy in melanoma patients. Science. 2018; 359(6371):97-103. doi: 10.1126/science.aan4236. PubMed PMID: 29097493.
15. Matson V, Fessler J, Bao R, Chongsuwat T, Zha Y, Alegre M L, et al. The commensal microbiome is associated with anti-PD-1 efficacy in metastatic melanoma patients. Science. 2018; 359(6371):104-8. doi: 10.1126/science.aao3290. PubMed PMID: 29302014.
16. Poutahidis T, Kleinewietfeld M, Erdman S E. Gut microbiota and the paradox of cancer immunotherapy. Frontiers in immunology. 2014; 5:157. Epub 2014/04/30. doi: 10.3389/fimmu.2014.00157. PubMed PMID: 24778636; PubMed Central PMCID: PMCPmc3985000.
17. Routy B, Le Chatelier E, Derosa L, Duong C P M, Alou M T, Daillere R, et al. Gut microbiome influences efficacy of PD-1-based immunotherapy against epithelial tumors. Science. 2018; 359(6371):91-7. doi: 10.1126/science.aan3706. PubMed PMID: 29097494.
18. Berg D, Clemente J C, Colombel J F. Can inflammatory bowel disease be permanently treated with short-term interventions on the microbiome? Expert review of gastroenterology & hepatology. 2015:1-15. Epub 2015/02/11. doi: 10.1586/17474124.2015.1013031. PubMed PMID: 25665875.
19. Collado M C, Rautava S, Isolauri E, Salminen S. Gut microbiota: a source of novel tools to reduce the risk of human disease? Pediatric research. 2015; 77(1-2):182-8. Epub 2014/10/22. doi: 10.1038/pr.2014.173. PubMed PMID: 25335085.
20. West C E, Renz H, Jenmalm M C, Kozyrskyj A L, Allen K J, Vuillermin P, et al. The gut microbiota and inflammatory noncommunicable diseases: associations and potentials for gut microbiota therapies. J Allergy Clin Immunol. 2015; 135(1):3-13; quiz 4. Epub 2015/01/09. doi: 10.1016/j.jaci.2014.11.012. PubMed PMID: 25567038.
21. Berin M C, Sampson H A. Mucosal immunology of food allergy. Current biology: C B. 2013; 23(9):R389-400. Epub 2013/05/11. doi: 10.1016/j.cub.2013.02.043. PubMed PMID: 23660362; PubMed Central PMCID: PMCPmc3667506.
22. Inoue Y, Shimojo N. Microbiome/microbiota and allergies. Seminars in immunopathology. 2015; 37(1):57-64. Epub 2014/10/19. doi: 10.1007/s00281-014-0453-5. PubMed PMID: 25326106.
23. Smits H H, Hiemstra P S, Prazeres da Costa C, Ege M, Edwards M, Garn H, et al. Microbes and asthma: Opportunities for intervention. J Allergy Clin Immunol. 2016; 137(3):690-7. doi: 10.1016/j.jaci.2016.01.004. PubMed PMID: 26947981.
24. Houttu N, Mokkala K, Laitinen K. Overweight and obesity status in pregnant women are related to intestinal microbiota and serum metabolic and inflammatory profiles. Clin Nutr. 2017. doi: 10.1016/j.clnu.2017.12.013. PubMed PMID: 29338886.
25. Iizumi T, Battaglia T, Ruiz V, Perez Perez G I. Gut Microbiome and Antibiotics. Arch Med Res. 2017. doi: 10.1016/j.arcmed.2017.11.004. PubMed PMID: 29221800.
26. Lopez-Contreras B E, Moran-Ramos S, Villarruel-Vazquez R, Macias-Kauffer L, Villamil-Ramirez H, Leon-Mimila P, et al. Composition of gut microbiota in obese and normal-weight Mexican school-age children and its association with metabolic traits. Pediatr Obes. 2017. doi: 10.1111/ijpo.12262. PubMed PMID: 29388394.
27. Okubo H, Nakatsu Y, Kushiyama A, Yamamotoya T, Matsunaga Y, Inoue M K, et al. Gut microbiota as a therapeutic target for metabolic disorders. Curr Med Chem. 2017. doi: 10.2174/0929867324666171009121702. PubMed PMID: 28990516.
28. Poutahidis T, Kleinewietfeld M, Smillie C, Levkovich T, Perrotta A, Bhela S, et al. Microbial reprogramming inhibits Western diet-associated obesity. PloS one. 2013; 8(7):e68596. Epub 2013/07/23. doi: 10.1371/journal.pone.0068596. PubMed PMID: 23874682; PubMed Central PMCID: PMCPmc3707834.
29. Dash S, Clarke G, Berk M, Jacka F N. The gut microbiome and diet in psychiatry: focus on depression. Current opinion in psychiatry. 2015; 28(1):1-6. Epub 2014/11/22. doi: 10.1097/yco.0000000000000117. PubMed PMID: 25415497.
30. Allen S J. The Potential of Probiotics to Prevent Clostridium difficile Infection. Infectious disease clinics of North America. 2015; 29(1):135-44. Epub 2015/02/14. doi: 10.1016/j.idc.2014.11.002. PubMed PMID: 25677707.
31. Mills J P, Rao K, Young V B. Probiotics for prevention of Clostridium difficile infection. Current opinion in gastroenterology. 2018; 34(1):3-10. doi: 10.1097/MOG.0000000000000410. PubMed PMID: 29189354.
32. Abraham B P, Quigley E M M. Probiotics in Inflammatory Bowel Disease. Gastroenterology clinics of North America. 2017; 46(4):769-82. doi: 10.1016/j.gtc.2017.08.003. PubMed PMID: 29173520.
33. Berin M C. Bugs versus bugs: probiotics, microbiome and allergy. Int Arch Allergy Immunol. 2014; 163(3):165-7. Epub 2014/02/01. doi: 10.1159/000357946. PubMed PMID: 24481028.
34. Schorpion A, Kolasinski S L. Can Probiotic Supplements Improve Outcomes in Rheumatoid Arthritis? Curr Rheumatol Rep. 2017; 19(11):73. doi: 10.1007/s11926-017-0696-y. PubMed PMID: 29094223.
35. Quigley J D, III, Wolfe T M. Effects of spray-dried animal plasma in calf milk replacer on health and growth of dairy calves. J Dairy Sci. 2003; 86(2):586-92.
36. Gionchetti P, Rizzello F, Campieri M. Probiotics in gastroenterology. CurrOpinGastroenterol. 2002; 18(2):235-9.
37. Homan E J, Bremel R D. A Role for Epitope Networking in Immunomodulation by Helminths. Frontiers in immunology. 2018; 9:1763. Epub 2018/08/16. doi: 10.3389/fimmu.2018.01763. PubMed PMID: 30108588; PubMed Central PMCID: PMCPMC6079203.
38. Gamonet C, Bole-Richard E, Delherme A, Aubin F, Toussirot E, Garnache-Ottou F, et al. New CD20 alternative splice variants: molecular identification and differential expression within hematological B cell malignancies. Exp Hematol Oncol. 2015; 5:7. Epub 2015/01/01. doi: 10.1186/s40164-016-0036-3. PubMed PMID: 26937306; PubMed Central PMCID: PMCPMC4774009.
39. Bajwa R, Cheema A, Khan T, Amirpour A, Paul A, Chaughtai S, et al. Adverse Effects of Immune Checkpoint Inhibitors (Programmed Death-1 Inhibitors and Cytotoxic T-Lymphocyte-Associated Protein-4 Inhibitors): Results of a Retrospective Study. J Clin Med Res. 2019; 11(4):225-36. Epub 2019/04/03. doi: 10.14740/jocmr3750. PubMed PMID: 30937112; PubMed Central PMCID: PMCPMC6436564.
40. Havel J J, Chowell D, Chan T A. The evolving landscape of biomarkers for checkpoint inhibitor immunotherapy. Nature reviews Cancer. 2019; 19(3):133-50. Epub 2019/02/14. doi: 10.1038/s41568-019-0116-x. PubMed PMID: 30755690.
41. Mandal R, Samstein R M, Lee K W, Havel J J, Wang H, Krishna C, et al. Genetic diversity of tumors with mismatch repair deficiency influences anti-PD-1 immunotherapy response. Science. 2019; 364(6439):485-91. Epub 2019/05/03. doi: 10.1126/science.aau0447. PubMed PMID: 31048490.
42. Gibney G T, Weiner L M, Atkins M B. Predictive biomarkers for checkpoint inhibitor-based immunotherapy. The lancet oncology. 2016; 17(12):e542-e51. Epub 2016/12/08. doi: 10.1016/S1470-2045(16)30406-5. PubMed PMID: 27924752; PubMed Central PMCID: PMCPMC5702534.
43. Bogen B, Malissen B, Haas W. Idiotope-specific T cell clones that recognize syngeneic immunoglobulin fragments in the context of class II molecules. European journal of immunology. 1986; 16(11):1373-8. Epub 1986/11/01. doi: 10.1002/eji.1830161110. PubMed PMID: 3096740.
44. Schaue D, McBride W H. T lymphocytes and normal tissue responses to radiation. Frontiers in oncology. 2012; 2:119. Epub 2012/10/11. doi: 10.3389/fonc.2012.00119. PubMed PMID: 23050243; PubMed Central PMCID: PMCPMC3445965.
45. Meyer C, Walker J, Dewane J, Engelmann F, Laub W, Pillai S, et al. Impact of irradiation and immunosuppressive agents on immune system homeostasis in rhesus macaques. Clin Exp Immunol. 2015; 181(3):491-510. Epub 2015/04/24. doi: 10.1111/cei.12646. PubMed PMID: 25902927; PubMed Central PMCID: PMCPMC4557385.
46. Gluzman-Poltorak Z, Vainstein V, Basile L A. Recombinant interleukin-12, but not granulocyte-colony stimulating factor, improves survival in lethally irradiated nonhuman primates in the absence of supportive care: evidence for the development of a frontline radiation medical countermeasure. Am J Hematol. 2014; 89(9):868-73. Epub 2014/05/24. doi: 10.1002/ajh.23770. PubMed PMID: 24852354.
47. Korber V, Yang J, Barah P, Wu Y, Stichel D, Gu Z, et al. Evolutionary Trajectories of IDH(WT) Glioblastomas Reveal a Common Path of Early Tumorigenesis Instigated Years ahead of Initial Diagnosis. Cancer Cell. 2019; 35(4):692-704 e12. Epub 2019/03/25. doi: 10.1016/j.ccell.2019.02.007. PubMed PMID: 30905762.
48. DeWitt W S, Lindau P, Snyder T M, Sherwood A M, Vignali M, Carlson C S, et al. A Public Database of Memory and Naive B-Cell Receptor Sequences. PloS one. 2016; 11(8):e0160853. doi: 10.1371/journal.pone.0160853. PubMed PMID: 27513338; PubMed Central PMCID: PMCPMC4981401.
49. Bashford-Rogers R J, Palser A L, Huntly B J, Rance R, Vassiliou G S, Follows G A, et al. Network properties derived from deep sequencing of human B-cell receptor repertoires delineate B-cell populations. Genome Res. 2013; 23(11):1874-84. doi: 10.1101/gr.154815.113. PubMed PMID: 23742949; PubMed Central PMCID: PMCPMC3814887.
50. Kipps T J, Stevenson F K, Wu C J, Croce C M, Packham G, Wierda W G, et al. Chronic lymphocytic leukaemia. Nat Rev Dis Primers. 2017; 3:16096. doi: 10.1038/nrdp.2016.96. PubMed PMID: 28102226; PubMed Central PMCID: PMCPMC5336551.
51. Puente X S, Bea S, Valdes-Mas R, Villamor N, Gutierrez-Abril J, Martin-Subero J I, et al. Non-coding recurrent mutations in chronic lymphocytic leukaemia. Nature. 2015; 526(7574):519-24. doi: 10.1038/nature14666. PubMed PMID: 26200345.
52. Valdes-Mas R, Gutierrez-Abril J, Puente X S, Lopez-Otin C. Chronic lymphocytic leukemia: looking into the dark side of the genome. Cell Death Differ. 2016; 23(1):7-9. doi: 10.1038/cdd.2015.155. PubMed PMID: 26611460; PubMed Central PMCID: PMCPMC4815973.
53. Khodadoust M S, Olsson N, Wagar L E, Haabeth O A, Chen B, Swaminathan K, et al. Antigen presentation profiling reveals recognition of lymphoma immunoglobulin neoantigens. Nature. 2017; 543(7647):723-7. doi: 10.1038/nature21433. PubMed PMID: 28329770.
54. Newman M E J. Power laws, Pareto distributions and Zipf's law. Contemporary Physics. 2005; 46(5):323-51.
55. Li W. Random Texts Exhibit Zipf's-Law-Like Word Frequency Distribution. IEEE Transactions on Information Theory, 1992; 38(6):1842-5.
56. Naumov Y N, Naumova E N, Hogan K T, Selin L K, Gorski J. A fractal clonotype distribution in the CD8+ memory T cell repertoire could optimize potential for immune responses. J Immunol. 2003; 170(8):3994-4001. Epub 2003/04/19. PubMed PMID: 12682227.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

What is claimed is:

1. A method for generating an output for diagnosing and monitoring the health and disease of an individual subject and designing an immunomodulatory intervention comprising:
determining a pattern of occurrence and frequency of T cell exposed motifs contained in a repertoire of proteins to which the individual is exposed as an indicator of the diversity of T cell stimulation provided by said repertoire of proteins, wherein said pattern is determined by:
collecting a biological sample containing said repertoire of proteins,
sequencing the proteins of the biological sample,
assembling a proteome from said repertoire of proteins,
extracting the T cell exposed amino acid motifs from said proteome,
determining the frequency of occurrence of each T cell exposed motif,
comparing the frequency of occurrence of each T cell exposed motif to the frequency distribution of T cell exposed motifs in a reference database of proteins selected from the group consisting of a human immunoglobulinome reference database, a human T cell receptor sequence reference database, a human proteome reference database, a human microbiome reference database, the proteome of one or more microorganisms other than the microbiome reference database, the allergome, an environmental organism reference database, and a tumor associated mutation reference database, and
generating a frequency pattern that identifies the unique T cell exposed motif distribution in said repertoire relative to the reference database; and
applying one or more unique features from the unique T cell exposed motif distribution of said frequency pattern to analyze or diagnose the health or disease status of said individual subject or to design or monitor an immunomodulatory intervention for that individual subject.

2. The method of claim 1 wherein said comparing the frequency of occurrence of each T cell exposed motif further comprises:
indexing each TCEM according to its frequency class in a reference data set of proteins, and
comparing the numbers of TCEM in each frequency class in said repertoire of proteins to which the individual is exposed relative to the numbers of TCEM in each frequency class in the reference dataset.

3. The method of claim 1 wherein said comparing the frequency of occurrence of each T cell exposed motif further comprises
indexing each TCEM according to its quantile score in a reference dataset of proteins, and
comparing the numbers of TCEM of each quantile score in said repertoire of proteins to which the individual is exposed relative to the reference dataset.

4. The method of claim 1 wherein said unique features of the unique T cell exposed motif distribution is a loss of TCEM diversity.

5. The method of claim 1 wherein said unique features of the unique T cell exposed motif distribution is a gain of TCEM diversity.

6. The method of claim 1 wherein said unique features of the unique T cell exposed motif distribution is a change in the number of TCEM of high frequency classes.

7. The method of claim 1 wherein said unique features of the unique T cell exposed motif distribution is a change in the number of TCEM of low frequency classes.

8. The method of claim 1 wherein said unique features of the unique T cell exposed motif distribution is a change in the number of a group of less than 1000 individual TCEM.

9. The method of claim 1 wherein said immunomodulatory intervention is selected from the group consisting of prophylactic or therapeutic vaccination, administration of CAR-T therapy, administration of a biopharmaceutical drug, administration of chemotherapy, administration of a checkpoint inhibitor, ablation of a population of B or T cells or their progenitors, transplant of B or T cells or their progenitors, radiation, and administration of a dietary supplement or probiotic.

10. The method of claim 1 wherein said application of the frequency pattern to analyze the health or disease of an individual is conducted prior to an immunomodulatory intervention.

11. The method of claim 1 wherein said application of the frequency pattern to analyze the health or disease of an individual is conducted after an immunomodulatory intervention to monitor the impact thereof on the frequency pattern.

12. The method of claim 1 wherein said application of the frequency pattern to analyze the health or disease of said individual subject is conducted as a routine monitoring to assess the diversity of the immune repertoire of said individual subject.

13. The method of claim 1, wherein said repertoire comprises at least 100 proteins.

14. The method of claim 1 wherein said individual subject is at risk of or suffering from a disease condition selected from the group consisting of cancer, autoimmunity, inflammatory diseases, allergies, infections, and a hematologic disease.

15. The method of claim 1 wherein said repertoire of proteins is comprised of the proteins present in a tissue sample.

16. The method of claim 15 wherein said tissue sample is from a tumor.

17. The method of claim 15 wherein said tissue sample is from normal tissue.

18. The method of claim 15 wherein the repertoires of proteins in normal and tumor tissue are compared to determine differences in the frequency distribution patterns of the T cell exposed motifs in each.

19. The method of claim 1 wherein said repertoire of proteins is comprised of the proteins of the microbiome of an individual subject.

20. The method of claim 1 wherein said repertoire of proteins is comprised of the proteins of bacteria from the group comprising bacteria intended to modify the human microbiome.
